SEQUENCE LISTING (184 sequences)

SEQ ID NO: 1 - protein, 261 aa, Artificial Sequence
  PSA protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 2 - DNA, 786 bp, Artificial Sequence
  PSA CDS
atgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctga

SEQ ID NO: 3 - protein, 10 aa, Artificial Sequence
  PSA 108-117
LTDAVKVMDL

SEQ ID NO: 4 - protein, 9 aa, Artificial Sequence
  PSA 146-154
KLQCVDLHV

SEQ ID NO: 5 - protein, 10 aa, Artificial Sequence
  PSA 154-163
VISNDVCAQV

SEQ ID NO: 6 - protein, 10 aa, Artificial Sequence
  PSA 141-150
FLTPKKLQCV

SEQ ID NO: 7 - protein, 9 aa, Artificial Sequence
  PSA 146-154 A1
ALQCVDLHV

SEQ ID NO: 8 - protein, 9 aa, Artificial Sequence
  PSA 146-154 A3
KLACVDLHV

SEQ ID NO: 9 - protein, 9 aa, Artificial Sequence
  PSA 146-154 A4
KLQAVDLHV

SEQ ID NO: 10 - protein, 9 aa, Artificial Sequence
  PSA 146-154 A5
KLQCADLHV

SEQ ID NO: 11 - protein, 9 aa, Artificial Sequence
  PSA 146-154 A6
KLQCVALHV

SEQ ID NO: 12 - protein, 9 aa, Artificial Sequence
  PSA 146-154 A7
KLQCVDAHV

SEQ ID NO: 13 - protein, 9 aa, Artificial Sequence
  PSA 146-154 A8
KLQCVDLAV

SEQ ID NO: 14 - protein, 120 aa, Artificial Sequence
  HCVR 2
EVQLVQSGAEVKRPGSSVKVSCKASGGTFSSYAINWVRRAPGQGLEWMGKIIPIPGITNYAQKFQDRVTFTADTSTNIAYMELSSLRSEDTAMYYCARSYKWGSSLVDAWGQGTLVTVSS

SEQ ID NO: 15 - protein, 120 aa, Artificial Sequence
  HCVR 3
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARNYYSQYWMMDLWGQGTLVTVSS

SEQ ID NO: 16 - protein, 118 aa, Artificial Sequence
  HCVR 5
QVQLVQSGAEVKKPGDSVKVSCKPSGYNFLNYGINWVRQAPGQGLEWMGWISTYTGNTNYAQKLQGRVTFTTDTSTSTAYMEMRSLRSDDTAVYYCARSSEYYTWDHWGQGTLVTVSS

SEQ ID NO: 17 - protein, 114 aa, Artificial Sequence
  HCVR 6
EVQLVQSGAEVKKPGASVKVSCKASGYTFTGYYIHWVRQAPGQGLEWMGGFDPEDGETIYAQKFQDRVTMTADTSTDTAYMELSSLRSEDTAVYYCARYGFDYWGQGTLVTVSS

SEQ ID NO: 18 - protein, 118 aa, Artificial Sequence
  HCVR 8
QVQLQQWGAGLLKPSETLSLTCAVKGGSFSDYYWSWIRQPPGKGLEWIGEINHSGGTNYNPSLKSRVTISVDTSKNQFSLKLLSVTAADTAVYYCARYNEYGSGYDKWGQGTLVTVSS

SEQ ID NO: 19 - protein, 118 aa, Artificial Sequence
  HCVR 9
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARSSQYYVWDSWGQGTLVTVSS

SEQ ID NO: 20 - protein, 123 aa, Artificial Sequence
  HCVR 10
QVQLVQSGAEVKKPGSSVKVSCKASGGTFSSYAISWVRQAPGQGLEWMGWINPNSGGTNYAQKFQGRVTMTRDTSISTAYMELSRLRSDDTAVYYCARWSYYYFQQFWSLDGWGQGTLVTVSS

SEQ ID NO: 21 - protein, 117 aa, Artificial Sequence
  HCVR 12
QMQLVQSGSEVKKPGASVKVSCKASGYTFTSYGISWVRQAPGQGLEWMGWISAYNGNTDYVQKLQGRVTMTTDTSTNTAYMELGSLGSDDTAVYYCARTNYNKYDIWGQGTLVTVSS

SEQ ID NO: 22 - protein, 114 aa, Artificial Sequence
  HCVR 13
QVQLVQSGAEVKKPGASVKVSCKASGYTFTSYYMHWVRQAPGQGLEWMGGFDPEDGETIYAQKFQGRVTMTEDTSTDTAYMGLSSLRSEDTAVYYCARYSYDYWGQGTLVTVSS

SEQ ID NO: 23 - protein, 114 aa, Artificial Sequence
  HCVR 14
EVQLVQSGAEVKKPGASVKVSCKASGYTFTGYFMHWVRQAPGQGLEWMGGFDPEDGETIYAQKFQGRVTMTEDTSTDTAYMELSSLRSEDTAVYYCARYSYDLWGQGTLVTVSS

SEQ ID NO: 24 - protein, 122 aa, Artificial Sequence
  HCVR 15
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARVSQPVYGSSTYDIWGQGTLVTVSS

SEQ ID NO: 25 - protein, 118 aa, Artificial Sequence
  HCVR 17
QMQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLVVPDAFDIWGQGTMVTVSS

SEQ ID NO: 26 - protein, 121 aa, Artificial Sequence
  HCVR 18
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWGSRGFLDAFDIWGQGTMVTVSS

SEQ ID NO: 27 - protein, 111 aa, Artificial Sequence
  LCVR 2
QSVLTQPPSASGTPGQRVTLSCSGSNSNIGSNTVNWYQQLPGTNPKLLIYSNNQRPSGVPDRFSGSKSGTSASLAISGLQSEDEADYYCATWDDSLNGPVFGGGTKLTVLG

SEQ ID NO: 28 - protein, 112 aa, Artificial Sequence
  LCVR 3
QSVLTQPPSVSGAPGQRVTISCTGSSSNFGAGYDVHWYQQLPGAAPKLLIYGDTNRPSGVPDRFSGSKSGTSASLAITGLQAEDEADYYCQSYDTSLSGSVFGGGTKLTVLG

SEQ ID NO: 29 - protein, 112 aa, Artificial Sequence
  LCVR 5
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGWVFGGGTKLTVLG

SEQ ID NO: 30 - protein, 110 aa, Artificial Sequence
  LCVR 6
QAVVTQEPSLTVSPGGTVTLTCASSTGAVTSGYYPNWFQLKPGQAPRALIYTTGKKHSWAPARFSGSLLGGKAALTLSGVQPEDEAEYYCLLYSGGVWVFGGGTKLTVLG

SEQ ID NO: 31 - protein, 111 aa, Artificial Sequence
  LCVR 8
QSVLTQPPSVSAAPGQKVTISCSGSSYNIGNNYVSWYQQLPGTAPKLLIYDNNKRPSGIPDRFSGSKSGTSATLGITGLQTGDEAEYYCGTWESSLSAYVFGTGTKVTVLG

SEQ ID NO: 32 - protein, 112 aa, Artificial Sequence
  LCVR 9
QSVVTQPPSVSGAPGQRVTISCTGSSSNFGAGYDVHWYQQLPGTAPKLLIYGNSNRPSGVPDRFSGSKSGTSASLAITGLQAEDEADYYCQSYDSSLSGWVFGGGTKLTVLG

SEQ ID NO: 33 - protein, 112 aa, Artificial Sequence
  LCVR 10
QAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGRWVFGGGTKLTVLG

SEQ ID NO: 34 - protein, 111 aa, Artificial Sequence
  LCVR 12
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSEVFGTGTKVTVLG

SEQ ID NO: 35 - protein, 110 aa, Artificial Sequence
  LCVR 13
QAVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAYVFGTGTKVTVLG

SEQ ID NO: 36 - protein, 111 aa, Artificial Sequence
  LCVR 14
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQWVFGGGTKLTVLG

SEQ ID NO: 37 - protein, 111 aa, Artificial Sequence
  LCVR 15
QAVLTQPPSASGTPGQRVTISCSGRSSNLGSNSVNWYQQVPGTAPKLLIFDNHQRPSGVPDRFSGSKSGTSASLAISGLRSEDETDYYCAAWDDSLNSVVFGGGTKLTVLG

SEQ ID NO: 38 - protein, 111 aa, Artificial Sequence
  LCVR 17
QSVVTQPPSVSAAPGQKVTISCSGSSSNIGNNYVSWYQQLPGTAPKLLIYDNYKRPSGIPDRFSGSKSGTSATLGITGLQTGDEADYYCGTWDSSLSAGVFGGGTKLTVLG

SEQ ID NO: 39 - protein, 111 aa, Artificial Sequence
  LCVR 18
QSVVTQPPSVSAAPGQRVTISCSGSSSNIGNNYVSWYQQLPGAAPRLLIYDNDKRPSGIPDRFSGSKSGTSATLGITGLQTGDEADYYCGTWDSSLSSGVFGGGTKLTVLG

SEQ ID NO: 40 - protein, 8 aa, Artificial Sequence
  HC-CDR1 2
GGTFSSYA

SEQ ID NO: 41 - protein, 8 aa, Artificial Sequence
  HC-CDR1 3
GFTFSSYA

SEQ ID NO: 42 - protein, 8 aa, Artificial Sequence
  HC-CDR1 5
GYNFLNYG

SEQ ID NO: 43 - protein, 8 aa, Artificial Sequence
  HC-CDR1 6
GYTFTGYY

SEQ ID NO: 44 - protein, 8 aa, Artificial Sequence
  HC-CDR1 8
GGSFSDYY

SEQ ID NO: 45 - protein, 8 aa, Artificial Sequence
  HC-CDR1 9
GYTFTSYG

SEQ ID NO: 46 - protein, 8 aa, Artificial Sequence
  HC-CDR1 10
GGTFSSYA

SEQ ID NO: 47 - protein, 8 aa, Artificial Sequence
  HC-CDR1 12
GYTFTSYG

SEQ ID NO: 48 - protein, 8 aa, Artificial Sequence
  HC-CDR1 13
GYTFTSYY

SEQ ID NO: 49 - protein, 8 aa, Artificial Sequence
  HC-CDR1 14
GYTFTGYF

SEQ ID NO: 50 - protein, 8 aa, Artificial Sequence
  HC-CDR1 15
GGTFSSYA

SEQ ID NO: 51 - protein, 8 aa, Artificial Sequence
  HC-CDR1 17
GYSFTSYW

SEQ ID NO: 52 - protein, 8 aa, Artificial Sequence
  HC-CDR1 18
GYSFTSYW

SEQ ID NO: 53 - protein, 8 aa, Artificial Sequence
  HC-CDR2 2
IIPIPGIT

SEQ ID NO: 54 - protein, 8 aa, Artificial Sequence
  HC-CDR2 3
ISGSGGST

SEQ ID NO: 55 - protein, 8 aa, Artificial Sequence
  HC-CDR2 5
ISTYTGNT

SEQ ID NO: 56 - protein, 8 aa, Artificial Sequence
  HC-CDR2 6
FDPEDGET

SEQ ID NO: 57 - protein, 7 aa, Artificial Sequence
  HC-CDR2 8
INHSGGT

SEQ ID NO: 58 - protein, 8 aa, Artificial Sequence
  HC-CDR2 9
ISAYNGNT

SEQ ID NO: 59 - protein, 8 aa, Artificial Sequence
  HC-CDR2 10
INPNSGGT

SEQ ID NO: 60 - protein, 8 aa, Artificial Sequence
  HC-CDR2 12
ISAYNGNT

SEQ ID NO: 61 - protein, 8 aa, Artificial Sequence
  HC-CDR2 13
FDPEDGET

SEQ ID NO: 62 - protein, 8 aa, Artificial Sequence
  HC-CDR2 14
FDPEDGET

SEQ ID NO: 63 - protein, 8 aa, Artificial Sequence
  HC-CDR2 15
IIPILGIA

SEQ ID NO: 64 - protein, 8 aa, Artificial Sequence
  HC-CDR2 17
IYPGDSDT

SEQ ID NO: 65 - protein, 8 aa, Artificial Sequence
  HC-CDR2 18
IYPGDSDT

SEQ ID NO: 66 - protein, 13 aa, Artificial Sequence
  HC-CDR3 2
ARSYKWGSSLVDA

SEQ ID NO: 67 - protein, 13 aa, Artificial Sequence
  HC-CDR3 3
ARNYYSQYWMMDL

SEQ ID NO: 68 - protein, 11 aa, Artificial Sequence
  HC-CDR3 5
ARSSEYYTWDH

SEQ ID NO: 69 - protein, 7 aa, Artificial Sequence
  HC-CDR3 6
ARYGFDY

SEQ ID NO: 70 - protein, 12 aa, Artificial Sequence
  HC-CDR3 8
ARYNEYGSGYDK

SEQ ID NO: 71 - protein, 11 aa, Artificial Sequence
  HC-CDR3 9
ARSSQYYVWDS

SEQ ID NO: 72 - protein, 16 aa, Artificial Sequence
  HC-CDR3 10
ARWSYYYFQQFWSLDG

SEQ ID NO: 73 - protein, 10 aa, Artificial Sequence
  HC-CDR3 12
ARTNYNKYDI

SEQ ID NO: 74 - protein, 7 aa, Artificial Sequence
  HC-CDR3 13
ARYSYDY

SEQ ID NO: 75 - protein, 7 aa, Artificial Sequence
  HC-CDR3 14
ARYSYDL

SEQ ID NO: 76 - protein, 15 aa, Artificial Sequence
  HC-CDR3 15
ARVSQPVYGSSTYDI

SEQ ID NO: 77 - protein, 11 aa, Artificial Sequence
  HC-CDR3 17
ARLVVPDAFDI

SEQ ID NO: 78 - protein, 14 aa, Artificial Sequence
  HC-CDR3 18
ARWGSRGFLDAFDI

SEQ ID NO: 79 - protein, 8 aa, Artificial Sequence
  LC-CDR1 2
NSNIGSNT

SEQ ID NO: 80 - protein, 9 aa, Artificial Sequence
  LC-CDR1 3
SSNFGAGYD

SEQ ID NO: 81 - protein, 9 aa, Artificial Sequence
  LC-CDR1 5
SSNIGAGYD

SEQ ID NO: 82 - protein, 9 aa, Artificial Sequence
  LC-CDR1 6
TGAVTSGYY

SEQ ID NO: 83 - protein, 8 aa, Artificial Sequence
  LC-CDR1 8
SYNIGNNY

SEQ ID NO: 84 - protein, 9 aa, Artificial Sequence
  LC-CDR1 9
SSNFGAGYD

SEQ ID NO: 85 - protein, 8 aa, Artificial Sequence
  LC-CDR1 10
SSNIGSNT

SEQ ID NO: 86 - protein, 9 aa, Artificial Sequence
  LC-CDR1 12
SSNIGAGYD

SEQ ID NO: 87 - protein, 9 aa, Artificial Sequence
  LC-CDR1 13
TGAVTSGYY

SEQ ID NO: 88 - protein, 9 aa, Artificial Sequence
  LC-CDR1 14
TGAVTSGYY

SEQ ID NO: 89 - protein, 8 aa, Artificial Sequence
  LC-CDR1 15
SSNLGSNS

SEQ ID NO: 90 - protein, 8 aa, Artificial Sequence
  LC-CDR1 17
SSNIGNNY

SEQ ID NO: 91 - protein, 8 aa, Artificial Sequence
  LC-CDR1 18
SSNIGNNY

SEQ ID NO: 92 - protein, 3 aa, Artificial Sequence
  LC-CDR2 2
SNN

SEQ ID NO: 93 - protein, 3 aa, Artificial Sequence
  SLC-CDR2 3
GDT

SEQ ID NO: 94 - protein, 3 aa, Artificial Sequence
  LC-CDR2 5
GNS

SEQ ID NO: 95 - protein, 3 aa, Artificial Sequence
  LC-CDR2 6
TTG

SEQ ID NO: 96 - protein, 3 aa, Artificial Sequence
  LC-CDR2 8
DNN

SEQ ID NO: 97 - protein, 3 aa, Artificial Sequence
  LC-CDR2 9
GNS

SEQ ID NO: 98 - protein, 3 aa, Artificial Sequence
  LC-CDR2 10
SNN

SEQ ID NO: 99 - protein, 3 aa, Artificial Sequence
  LC-CDR2 12
GNS

SEQ ID NO: 100 - protein, 3 aa, Artificial Sequence
  LC-CDR2 13
STS

SEQ ID NO: 101 - protein, 3 aa, Artificial Sequence
  LC-CDR2 14
STS

SEQ ID NO: 102 - protein, 3 aa, Artificial Sequence
  LC-CDR2 15
DNH

SEQ ID NO: 103 - protein, 3 aa, Artificial Sequence
  LC-CDR2 17
DNY

SEQ ID NO: 104 - protein, 3 aa, Artificial Sequence
  LC-CDR2 18
DND

SEQ ID NO: 105 - protein, 11 aa, Artificial Sequence
  LC-CDR3 2
ATWDDSLNGPV

SEQ ID NO: 106 - protein, 11 aa, Artificial Sequence
  LC-CDR3 3
QSYDTSLSGSV

SEQ ID NO: 107 - protein, 11 aa, Artificial Sequence
  LC-CDR3 5
QSYDSSLSGWV

SEQ ID NO: 108 - protein, 9 aa, Artificial Sequence
  LC-CDR3 6
LLYSGGVWV

SEQ ID NO: 109 - protein, 11 aa, Artificial Sequence
  LC-CDR3 8
GTWESSLSAYV

SEQ ID NO: 110 - protein, 11 aa, Artificial Sequence
  LC-CDR3 9
QSYDSSLSGWV

SEQ ID NO: 111 - protein, 12 aa, Artificial Sequence
  LC-CDR3 10
AAWDDSLNGRWV

SEQ ID NO: 112 - protein, 10 aa, Artificial Sequence
  LC-CDR3 12
QSYDSSLSEV

SEQ ID NO: 113 - protein, 9 aa, Artificial Sequence
  LC-CDR3 13
LLYYGGAYV

SEQ ID NO: 114 - protein, 10 aa, Artificial Sequence
  LC-CDR3 14
LLYYGGAQWV

SEQ ID NO: 115 - protein, 11 aa, Artificial Sequence
  LC-CDR3 15
AAWDDSLNSVV

SEQ ID NO: 116 - protein, 11 aa, Artificial Sequence
  LC-CDR3 17
GTWDSSLSAGV

SEQ ID NO: 117 - protein, 11 aa, Artificial Sequence
  LC-CDR3 18
GTWDSSLSSGV

SEQ ID NO: 118 - protein, 8 aa, Artificial Sequence
  HC-CDR1 consensus
  VARIANT 2: Xaa = G or Y
  VARIANT 3: Xaa = S or T
  VARIANT 5: Xaa = S or T
  VARIANT 8: Xaa = A or G
GXXFXSYX

SEQ ID NO: 119 - protein, 8 aa, Artificial Sequence
  HC-CDR2 consensus
  VARIANT 2, 4, 5, 7: Xaa = Any Amino Acid
IXPXXGXT

SEQ ID NO: 120 - protein, 5 aa, Artificial Sequence
  HC-CDR3 consensus
  VARIANT 3, 4: Xaa = Any Amino Acid
ARXXY

SEQ ID NO: 121 - protein, 8 aa, Artificial Sequence
  LC-CDR1 consensus
  VARIANT 1: Xaa = S or T
  VARIANT 4: Xaa = F or I
  VARIANT 6: Xaa = A or N or S
  VARIANT 7: Xaa = G or N
XSNXGXXY

SEQ ID NO: 122 - protein, 10 aa, Artificial Sequence
  LC-CDR3 consensus 1
  VARIANT 1: Xaa = A or G
  VARIANT 2: Xaa = A or T
  VARIANT 4: Xaa = D or S
  VARIANT 7: Xaa = N or S
  VARIANT 8: Xaa = A or G
  VARIANT 9: Xaa = Any Amino Acid
XXWXSLXXXV

SEQ ID NO: 123 - protein, 6 aa, Artificial Sequence
  LC-CDR3 consensus 2
  VARIANT 4: Xaa = Any Amino Acid
LLYXGG

SEQ ID NO: 124 - protein, 11 aa, Artificial Sequence
  LC-CDR3 consensus 3
  VARIANT 5: Xaa = S or T
  VARIANT 10: Xaa = Any Amino Acid
QSYDXSLSGXV

SEQ ID NO: 125 - protein, 9 aa, Artificial Sequence
  A2E-1
LLDVPTAAV

SEQ ID NO: 126 - protein, 9 aa, Artificial Sequence
  A2E-2
TLWVDPYEV

SEQ ID NO: 127 - protein, 9 aa, Artificial Sequence
  A2E-3
FLLDHLKRV

SEQ ID NO: 128 - protein, 10 aa, Artificial Sequence
  A2E-4
LLLDVPTAAV

SEQ ID NO: 129 - protein, 13 aa, Artificial Sequence
  A2E-5
VLFRGGPRGLLAV

SEQ ID NO: 130 - protein, 9 aa, Artificial Sequence
  A2E-6
SLLPAIVEL

SEQ ID NO: 131 - protein, 9 aa, Artificial Sequence
  A2E-7
YLLPAIVHI

SEQ ID NO: 132 - protein, 9 aa, Artificial Sequence
  A2E-8
FLLPTGAEA

SEQ ID NO: 133 - protein, 10 aa, Artificial Sequence
  A2E-9
LLDPKLCYLL

SEQ ID NO: 134 - protein, 10 aa, Artificial Sequence
  A2E-10
SLPHFHHPET

SEQ ID NO: 135 - protein, 10 aa, Artificial Sequence
  A2E-11
MLLSVPLLLG

SEQ ID NO: 136 - protein, 11 aa, Artificial Sequence
  A2E-13
LLYDMVCGDIP

SEQ ID NO: 137 - protein, 11 aa, Artificial Sequence
  A2E-14
LLLDVPTAAVQ

SEQ ID NO: 138 - protein, 12 aa, Artificial Sequence
  A2E-15
LLLDVPTAAVQA

SEQ ID NO: 139 - protein, 14 aa, Artificial Sequence
  A2E-16
VLFRGGPRGLLAVA

SEQ ID NO: 140 - protein, 9 aa, Artificial Sequence
  A2E-17
MVDGTLLLL

SEQ ID NO: 141 - protein, 10 aa, Artificial Sequence
  A2E-19
YMAPEILMRS

SEQ ID NO: 142 - protein, 10 aa, Artificial Sequence
  A2E-21
FIYNADLMNC

SEQ ID NO: 143 - protein, 11 aa, Artificial Sequence
  A2E-23
KQYESVLMVSI

SEQ ID NO: 144 - protein, 9 aa, Artificial Sequence
  AFP158
FMNKFIYEI

SEQ ID NO: 145 - protein, 122 aa, Artificial Sequence
  HCVR 20
EVQLVQSGAEVKKPGESLKISCKGSGYSFTSYRIGWVRQMPGKGLEWMGIIYPGDSDTRYSPSFQGQVTISADKSISTAYLQWSSLKASDTAMYYCARWGLSWDGWGVTDYWGQGTLVTVSS

SEQ ID NO: 146 - protein, 114 aa, Artificial Sequence
  HCVR 21
QVQLVQSGAEVKKPGASVKVSCKASGYTFTSYYMHWVRQAPGKGLEWMGGFDPEDGETIYAQKFQGRVTMTEDTSTDTAYMELSSLRSEDTAVYYCARYNYDTWGQGTLVTVSS

SEQ ID NO: 147 - protein, 117 aa, Artificial Sequence
  HCVR 22
EVQLVQSGAEVKKPGESLKISCKGSGYSFTSYWIGWVRQMPGKGLEWMGRIDPSDSYTNYSPSFQGHVTISADKSISTAYLQWSSLKASDTAMYYCARSFGAGYDSWGQGTLVTVSS

SEQ ID NO: 148 - protein, 114 aa, Artificial Sequence
  HCVR 24
QVQLVQSGAEVKKPGASVKVSCKASGYTFTSYYMHWVRQAPGKGLEWMGGFDPEDGETIYAQKFQGRVTMTEDTSTDTAYMELSSLRSEDTAVYYCARYPWDHWGQGTLVTVSS

SEQ ID NO: 149 - protein, 119 aa, Artificial Sequence
  HCVR 26
QVQLVQSGAEVKKPGASVKVSCKTSGYTFTNYGISWVRQAPGQGLEWMGWISAYNGNTNYAQNLQGRVTMTTDTSTSTAYMELRSLRSDDTAVYYCARSSYYGYLSDGWGQGTLVTVSS

SEQ ID NO: 150 - protein, 108 aa, Artificial Sequence
  LCVR 20
DIQLTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKR

SEQ ID NO: 151 - protein, 110 aa, Artificial Sequence
  LCVR 21
QAVVTQEPSLTVSPGGTVTLTCASSTGTVTSTYYPNWFQQKPGQAPRALIYSTSNRHSWTPARFSGSLLGGKAALTVSGVQPDDEAEYYCLVFYGGVWVFGGGTKLTVLG

SEQ ID NO: 152 - protein, 111 aa, Artificial Sequence
  LCVR 22
QSALTQPASVSGSPGQSITISCTGTSSDVGGYNYVSWYQQHPGKAPKLMIYDVSKRPSGVSNRFSGSKSGNTASLTISGLQAEDEADYYCSSYTSSSRYVFGTGTKVTVLG

SEQ ID NO: 153 - protein, 110 aa, Artificial Sequence
  LCVR 24
QAVVTQEPSLTVSPGGTVTLTCASSTGAVTSGYYPNWFQQKPGQAPRPLIYSTNNKHSWTPARFSGSLLGGKAALTLSGVQPEDEAEYYCLLYYGGQGVFGGGTKLTVLG

SEQ ID NO: 154 - protein, 112 aa, Artificial Sequence
  LCVR 26
LPVLTQPPSASGTPGQRVTISCSGSSSNIGTNYVYWYQQLPGTAPKLLIYSNNQRPSGVPDRFSGSKSGTSASLAISGLRSEDEADYYCAAWDDSLSGLYVFGTGTKVTVLG

SEQ ID NO: 155 - protein, 8 aa, Artificial Sequence
  HC-CDR1 20
GYSFTSYR

SEQ ID NO: 156 - protein, 8 aa, Artificial Sequence
  HC-CDR1 26
GYTFTNYG

SEQ ID NO: 157 - protein, 8 aa, Artificial Sequence
  HC-CDR2 22
IDPSDSYT

SEQ ID NO: 158 - protein, 15 aa, Artificial Sequence
  HC-CDR3 20
ARWGLSWDGWGVTDY

SEQ ID NO: 159 - protein, 7 aa, Artificial Sequence
  HC-CDR3 21
ARYNYDT

SEQ ID NO: 160 - protein, 10 aa, Artificial Sequence
  HC-CDR3 22
ARSFGAGYDS

SEQ ID NO: 161 - protein, 7 aa, Artificial Sequence
  HC-CDR3 24
ARYPWDH

SEQ ID NO: 162 - protein, 12 aa, Artificial Sequence
  HC-CDR3 26
ARSSYYGYLSDG

SEQ ID NO: 163 - protein, 6 aa, Artificial Sequence
  LC-CDR1 20
QSISSY

SEQ ID NO: 164 - protein, 9 aa, Artificial Sequence
  LC-CDR1 21
TGTVTSTYY

SEQ ID NO: 165 - protein, 9 aa, Artificial Sequence
  LC-CDR1 22
SSDVGGYNY

SEQ ID NO: 166 - protein, 8 aa, Artificial Sequence
  LC-CDR1 26
SSNIGTNY

SEQ ID NO: 167 - protein, 3 aa, Artificial Sequence
  LC-CDR2 20
AAS

SEQ ID NO: 168 - protein, 3 aa, Artificial Sequence
  LC-CDR2 22
DVS

SEQ ID NO: 169 - protein, 3 aa, Artificial Sequence
  LC-CDR2 24
STN

SEQ ID NO: 170 - protein, 9 aa, Artificial Sequence
  LC-CDR3 20
QQSYSTPFT

SEQ ID NO: 171 - protein, 9 aa, Artificial Sequence
  LC-CDR3 21
LVFYGGVWV

SEQ ID NO: 172 - protein, 10 aa, Artificial Sequence
  LC-CDR3 22
SSYTSSSRYV

SEQ ID NO: 173 - protein, 9 aa, Artificial Sequence
  LC-CDR3 24
LLYYGGQGV

SEQ ID NO: 174 - protein, 12 aa, Artificial Sequence
  LC-CDR3 26
AAWDDSLSGLYV

SEQ ID NO: 175 - protein, 5 aa, Artificial Sequence
  Linker 1
GGGGS

SEQ ID NO: 176 - protein, 21 aa, Artificial Sequence
  Linker 2
SRGGGGSGGGGSGGGGSLEMA

SEQ ID NO: 177 - protein, 222 aa, Artificial Sequence
  CD28/CD3zeta
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - protein, 225 aa, Artificial Sequence
  4-1BB/CD3zeta
TGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 179 - DNA, 354 bp, Artificial Sequence
  HCVR 5
caggtccagctggtgcagtctggagctgaggtgaagaagcctggggattcagtgaaggtctcctgcaagccttctggttacaattttctcaactatggtatcaactgggtgcgacaggcccctggacaagggcttgagtggatgggatggattagcacttacaccggtaacacaaactatgcacagaagctgcagggcagagtcaccttcaccacagacacatccacgagcacagcctacatggagatgaggagcctgagatctgacgacacggccgtgtattactgtgcgcgctcttctgaatactacacttgggatcattggggtcaaggtactctggtgaccgtctcctca

SEQ ID NO: 180 - DNA, 336 bp, Artificial Sequence
  LCVR 5
cagtctgtgttgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttgggtgttcggcggagggaccaagctgaccgtcctaggt

SEQ ID NO: 181 - protein, 6 aa, Artificial Sequence
  HC-CDR3 consensus
  VARIANT 4: Xaa = Any Amino Acid
  VARIANT 5: Xaa = F or W or Y
ARYXXD

SEQ ID NO: 182 - protein, 7 aa, Artificial Sequence
  LC-CDR3 consensus 1
  VARIANT 1: Xaa = A or G
  VARIANT 2: Xaa = Any Amino Acid
  VARIANT 4: Xaa = D or E
  VARIANT 5: Xaa = Any Amino Acid
XXWXXSL

SEQ ID NO: 183 - protein, 6 aa, Artificial Sequence
  LC-CDR3 consensus 2
  VARIANT 2: Xaa = L or V
  VARIANT 3: Xaa = F or Y
  VARIANT 4: Xaa = Any Amino Acid
LXXXGG

SEQ ID NO: 184 - protein, 6 aa, Artificial Sequence
  LC-CDR3 consensus 3
  VARIANT 1: Xaa = S or T
  VARIANT 2: Xaa = W or Y
  VARIANT 3: Xaa = Any Amino Acid
  VARIANT 4: Xaa = S or T
XXXXSL

(19) United States
(12) Patent Application Publication
(10) Pub. No.: US 2020/0087400 A1
(43) Pub. Date: Mar. 19, 2020

(54) CONSTRUCTS TARGETING PSA PEPTIDE/MHC COMPLEXES AND USES THEREOF

(71) Applicant: EUREKA THERAPEUTICS, INC.

(72) Inventors: Hong LIU, El Sobrante, CA (US); Jingyi XIANG, Walnut Creek, CA (US); Yiyang XU, Pleasanton, CA (US); Vivien Wai-Fan CHAN, Emeryville, CA (US)

(73) Assignees: EUREKA THERAPEUTICS, INC., Emeryville, CA (US); EUREKA THERAPEUTICS, INC., Emeryville, CA (US)

(21) Appl. No.: 15/746,367

(22) PCT Filed: Jul. 22, 2016
(86) PCT No.: PCT/US2016/043753

§ 371 Date: Jan. 19, 2018

Related U.S. Application Data
(60) Provisional application No. 62/195,706, filed on Jul. 22, 2015.

(51) Int. Cl. C07K 16/28; C07K 16/30

(52) U.S. Cl. CPC C07K 16/2833; C07K 16/3069; C07K 2317/56; C07K 2317/32; C07K 2317/55; C07K 2317/21

(57) ABSTRACT

The present application provides constructs comprising an antibody moiety that specifically binds to a complex comprising a PSA peptide and an MHC class I protein. Also provided are methods of making and using these constructs.

### CROSS-REFERENCE TO RELATED APPLICATIONS

This application claims priority to U.S. Provisional Application No. 62/195,706, filed on Jul. 22, 2015, which is hereby incorporated by reference in its entirety.

### FIELD OF THE INVENTION

This invention pertains to antibody constructs that specifically bind MHC molecules complexed with PSA peptides, and uses thereof including treating and diagnosing diseases.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 750042000540SEQLIST.TXT, date recorded: Jul. 21, 2016, size: 71 KB).

### BACKGROUND OF THE INVENTION

Cell surface proteins constitute only a small fraction of the cellular proteins and these proteins are often not tumor-specific. Because of the inability to easily penetrate cells, marketed therapeutic monoclonal antibodies (mAbs) recognize these cell surface proteins, most of which are lineage or differentiation antigens (Milenic, E. D., Curr. Pharm. Des. 8:1794-1764, 2002; Grillo-Lopez. A. J., Expert Rev. Anticancer Ther. 2(3):323-329, 2002; Jones, K. L. & Buzdat. A. U., Lancet Oncol. 10(12):1179-1187, 2009). In contrast, mutated or oncogenic tumor-associated proteins are typically nuclear, cytoplasmic or secreted, which are currently best addressed either by small molecule drugs, or in the case of secreted proteins, hardly addressed as anti-cancer drug targets (Reddy et al., Expert Opin. Ther. Targets 3:313-324, 2012; Takeuchi, K. & Ito, F., Biol. Pharm. Bull. 34(12):1774-1780; Roychowdhury, S. & Talpaz, M., Blood Rev. 6:279-290, 2011). However, most intracellular proteins can be proteosomally degraded, processed and presented by MHC molecules on the cell surface as T cell peptide epitopes in the context of MHC molecules that are recognized by T cell receptors (TCRs) (Morris et al., Blood Rev. 20:61-69, 2006; Konnig, R., Curr. Opin. Immunol. 14(1):75-83, 2002). Therefore, generating therapeutic mAbs that recognize the secreted or intracellular tumor antigen-derived peptide/MHC complexes on the cell surface will take advantage of the enhanced specificity and therapeutic potency offered by mAbs. Recent advances in using phage display to generate mAbs have made it possible to select agents with exquisite specificity against defined epitopes from large antibody repertoires. A number of such mAbs specific for solid tumor antigens, in the context of HLA-A01 and HLA-A02, have been successfully selected from phage display libraries (Noy et al., Expert Rev. Anticancer Ther. 5(3):523-536, 2005; Chames et al., Proc. Natl. Acad. Sci. USA 97:7969-7974, 2000; Held et al., Eur. J. Immunol. 34:2919-2929, 2004; Lev et al., Cancer Res. 62:3184-3194, 2002; Klechevsky et al., Cancer Res. 68(15):6360-6367, 2008). More recently, a human mAb specific for human WT1/HLA-A02 complex, a well-described T cell epitope, has been shown to inhibit multiple cancer cell lines and primary cancer cells via Fc-mediated effector cell function (Dao et al., Sci. Trans. Med. 5:176ra33, 2013; Veomett et al., Clin. Cancer Res. doi: 10.1158/1078-0432, 2014) in cellular assays and in in vivo models.

Prostate cancer is the second most common cancer and the second leading cause of cancer-related death in American men. There are over 27,000 deaths from prostate cancer every year in Unite State (American Cancer Society). Between 35-61% of prostate cancer patients undergoing radical prostatectomy or radical radiotherapy eventually relapse. These patients may respond transiently to androgen deprivation therapy, but a majority will subsequently progress to hormone-refractory disease for which curative systemic therapies are lacking (Perambakam S, et al., Clin. Dev. Immunol. 2010; Epub 2011 Jan. 5).

The prostate-specific antigen (PSA), also known as kallikrein-related peptidase 3 (KLK3), a 34 kDa single-chain glycoprotein, is a kallikrein-like serine protease. It is normally produced by prostate epithelial cells and secreted into the prostatic ducts. PSA is also expressed at high levels in benign prostatic hypertrophy and prostate cancer of all grades and stages. The paraurethral and perianal glands, placenta, breast (including breast cancer) and thyroid may also express PSA at very low levels. The physiological role of PSA is mainly to liquefy the seminal fluid (Lilja H. Urology. 62(5 Suppl1):27-33, 2003). In healthy male individuals, PSA is tightly confined within the prostatic glandular lumen and only trivial amount is leaked into circulation. On average, PSA blood concentration is about 0.6 ng/ml in healthy male adults. However, in prostate cancer, the disordered glandular architecture causes increased amount of PSA to diffuse into the circulation. Due to the highly restricted and prostate-specific expression profile, blood PSA measurement is regarded as the primary screening and prognostic marker for prostate cancer (Lilja H. et al., Nat. Rev. Cancer. 8(4):268-78, 2008; Erratum in: Nat. Rev. Cancer. 8(5):403, 2008).

Prostate cancer is a suitable target for immunotherapy. It presents a variety of tumor-associated antigens such as PSA, prostatic acid phosphatase (PAP) and prostate-specific membrane antigen (PSMA), all of which have been shown to produce certain level of clinical responses through immunogenicity. Prostate cancer also has a relatively slow growth rate that may allow the immune system the necessary time to produce a response. Finally, the prostate is a dispensable organ, and therefore PSA-directed therapy is expected to be well-tolerated. Multiple immunotherapeutic reagents are being studied for the treatment of prostate cancer. For PSA, a non-cell surface protein, immunotherapeutic approaches have been mainly limited to DNA, protein or peptide vaccines. The clinical trials of PSA-based vaccines, such as Ad/PSA, Fowlpox PSA, PSA₁₅₄₋₁₆₃ peptide and PSA₁₄₆₋₁₅₄ peptide, have shown no serious adverse effects. Some patients obtained clinical benefits from the PSA vaccine therapies, showing greater overall survival time compared with control treatment (Kouiavskaia D V, et al., J. Immunother. 32(6):655-66, 2009; Noguchi M. et al., BMC Cancer. 13:613, 2013; DiPaola R S, et al., Eur. Urol, pii: S0302-2838(14)01265-2, 2014). However, antibody-based or adoptive T cell therapies against PSA have not been extensively explored.

Although PSA is mainly regarded as a tumor marker for prostate cancer, increased PSA expression has also been observed in several other cancer types. For example, studies have indicated that PSA levels in circulation were significantly higher in cases with malignant breast tumors in women (Razavi S H. et al., Int. J. Prev. Med. 6:15, 2015; Black M H, et al., Clin. Cancer Res. 6(2):467-73, 2000). A rare single nucleotide polymorphism (SNP) located within the androgen response element V of PSA was associated with poor 5-year survival in ovarian cancer (O'Mara T A, et al., Twin Res. Hum. Genet. 14(4):323-7, 2011). Meanwhile, a comprehensive mRNA profiling study reported that PSA mRNA expression was elevated in ovarian serous tumors (Gilks C B, et al., Gynecol. Oncol. 96(3):684-94, 2005). Several lung cancer cell lines, including HSRRB and BEN, also exhibited abnormally high levels of PSA at the mRNA level (Barretina J, et al., Nature. 483(7391):603-7, 2012).

A series of PSA-derived peptides restricted by MHC I and MHC II molecules, including PSA₁₄₆₋₁₅₄, have been reported (Klyushnenkova E N. et al., Clin. Cancer Res. 11(8):2853-61, 2005; Elkord E, et al., Int. Immunol. 17(10):1315-25, 2005). PSA-derived peptide₁₄₆₋₁₅₄, KLQCVDLHV (PSA146), is a CD8+ T cell epitope presented by HLA-A*02:01. Cytotoxic T cells (CTLs) recognizing PSA146/HLA-A*02:01 were able to kill PSA-expressing epithelial prostate cancer cell line LNCaP specifically (Elkord E, et al., supra). Patients treated with a PSA146-based peptide vaccine developed strong vaccine-specific delayed-type hypersensitivity skin responses, tetramer or IFN-gamma responses. A trend towards greater overall survival was also detected in patients with high-risk, hormone-sensitive prostate cancer who developed PSA146-specific T-cell immunity following vaccination in the same study (Perambakam S. et al., supra).

The disclosures of all publications, patents, patent applications and published patent applications referred to herein are hereby incorporated herein by reference in their entirety.

### BRIEF SUMMARY OF THE INVENTION

The present application in one aspect provides constructs (such as isolated constructs) that bind to a complex comprising a PSA peptide and an MHC class I protein (referred to herein as a “PSA/MHC class I complex.” or “PMC”). In some embodiments, the constructs (“anti-PMC constructs”) comprise an antibody moiety (referred to herein as an “anti-PMC antibody moiety”) that specifically binds to a complex comprising a PSA peptide and an MHC class I protein.

Thus, in some embodiments, there is provided an anti-PMC construct (such as an isolated anti-PMC construct) comprising an antibody moiety that specifically binds to a complex comprising a PSA peptide and an MHC class I protein. In some embodiments, the PSA/MHC complex is present on a cell surface. In some embodiments, the PSA/MHC complex is present on the surface of a cancer cell.

In some embodiments, the anti-PMC construct comprises an antibody moiety that specifically binds to a complex comprising a PSA peptide and an MHC class I protein, wherein the MHC class I protein is HLA-A. In some embodiments, the MHC class I protein is HLA-A02. In some embodiments, the MHC class I protein is the HLA-A*02:01 subtype of the HLA-A02 allele.

In some embodiments, according to any of the anti-PMC constructs (such as isolated anti-PMC constructs) described above, the anti-PMC construct comprises an antibody moiety that specifically binds to a complex comprising a PSA peptide and an MHC class I protein, wherein the antibody moiety cross-reacts with a complex comprising the PSA peptide and a second MHC class I protein having a different HLA allele than the MHC class I protein. In some embodiments, the antibody moiety cross-reacts with at least one complex comprising a variant of the PSA peptide comprising one amino acid substitution (such as a conservative amino acid substitution) and the MHC class I protein.

In some embodiments, according to any of the anti-PMC constructs (such as isolated anti-PMC constructs) described above, the anti-PMC construct comprises an antibody moiety that specifically binds to a complex comprising a PSA peptide and an MHC class I protein, wherein the PSA peptide is about 8 to about 12 (such as about any of 8, 9, 10, 11, or 12) amino acids in length. In some embodiments, the PSA peptide has an amino acid sequence selected from the group consisting of SEQ ID NOs: 3-13. In some embodiments, the PSA peptide has the amino acid sequence KLQCVDLHV (SEQ ID NO: 4).

In some embodiments, according to any of the anti-PMC constructs (such as isolated anti-PMC constructs) described above, the anti-PMC construct comprises an antibody moiety that specifically binds to a complex comprising a PSA peptide and an MHC class I protein, wherein the antibody moiety is a full-length antibody, a Fab, a Fab′, a (Fab′)2, an Fv, or a single chain Fv (scFv). In some embodiments, the antibody moiety is fully human, semi-synthetic with human antibody framework regions, or humanized.

In some embodiments, according to any of the anti-PMC constructs (such as isolated anti-PMC constructs) described above, the anti-PMC construct comprises an antibody moiety that specifically binds to a complex comprising a PSA peptide and an MHC class I protein, wherein the antibody moiety binds to the PSA/MHC class I complex with an equilibrium dissociation constant (K_{d}) between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the isolated anti-PMC construct binds to the PSA/MHC class I complex with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values).

In some embodiments, the anti-PMC construct comprises an antibody moiety that specifically binds to a complex comprising a PSA peptide and an MHC class I protein, wherein the antibody moiety comprises: i) a heavy chain variable domain comprising a heavy chain complementarity determining region (HC-CDR) 1 comprising the amino acid sequence of G-G/Y-S/T-F-S/T-S-Y-A/G (SEQ ID NO: 118), or a variant thereof comprising up to about 3 (such as about any of 1, 2, or 3) amino acid substitutions, an HC-CDR2 comprising the amino acid sequence of I-X-P-X-X-G-X-T (SEQ ID NO: 119), or a variant thereof comprising up to about 3 (such as about any of 1, 2, or 3) amino acid substitutions, and an HC-CDR3 comprising the amino acid sequence of A-R-X-X-Y (SEQ ID NO: 120) or A-R-Y-X-F/W/Y-D (SEQ ID NO: 181), or a variant thereof comprising up to about 3 (such as about any of 1, 2, or 3) amino acid substitutions; and ii) a light chain variable domain comprising a light chain complementarity determining region (LC-CDR) 1 comprising the amino acid sequence of S/T-S-N-F/I-G-A/N/S-G/N-Y (SEQ ID NO: 121), or a variant thereof comprising up to about 3 (such as about any of 1, 2, or 3) amino acid substitutions, and an LC-CDR3 comprising the amino acid sequence of A/G-A/T-W-D/S-S-L-N/S-A/G-X-V (SEQ ID NO: 122), L-L-Y-X-G-G (SEQ ID NO: 123), Q-S-Y-D-S/T-S-L-S-G-X-V (SEQ ID NO: 124), A/G-X-W-D/E-X-S-L (SEQ ID NO: 182), L-L/V-F/Y-X-G-G (SEQ ID NO: 183), or S/T-W/Y-X-S/T-S-L (SEQ ID NO: 184), or a variant thereof comprising up to about 3 (such as about any of 1, 2, or 3) amino acid substitutions, wherein X can be any amino acid.

In some embodiments, the anti-PMC construct comprises an antibody moiety that specifically binds to a complex comprising a PSA peptide and an MHC class I protein, wherein the antibody moiety comprises: i) a heavy chain variable domain comprising an HC-CDR1 comprising (and in some embodiments consisting of) the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising (and in some embodiments consisting of) the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising (and in some embodiments consisting of) the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising (and in some embodiments consisting of) the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising (and in some embodiments consisting of) the amino acid sequence of any one of SEQ ID NOs: 92-104 and 167-169, or a variant thereof comprising up to about 3 (such as about any of 1, 2, or 3) amino acid substitutions, and an LC-CDR3 comprising (and in some embodiments consisting of) the amino acid sequence of any one of SEQ ID NOs: 105-117 and 170-174, or a variant thereof comprising up to about 5 (such as about any of 1, 2, 3, 4, or 5) amino acid substitutions.

In some embodiments, the anti-PMC construct comprises an antibody moiety that specifically binds to a complex comprising a PSA peptide and an MHC class I protein, wherein the antibody moiety comprises: i) a heavy chain variable domain comprising an HC-CDR1 comprising (and in some embodiments consisting of) the amino acid sequence of any one of SEQ ID NOs: 40-52, 155, and 156, an HC-CDR2 comprising (and in some embodiments consisting of) the amino acid sequence of any one of SEQ ID NOs: 53-65 and 157, and an HC-CDR3 comprising (and in some embodiments consisting of) the amino acid sequence of any one of SEQ ID NOs: 66-78 and 158-162; or a variant thereof comprising up to about 5 (such as about any of 1, 2, 3, 4, or 5) amino acid substitutions in the HC-CDR regions; and ii) a light chain variable domain comprising an LC-CDR1 comprising (and in some embodiments consisting of) the amino acid sequence of any one of SEQ ID NOs: 79-91 and 163-166, an LC-CDR2 comprising (and in some embodiments consisting of) the amino acid sequence of any one of SEQ ID NOs: 92-104 and 167-169, and an LC-CDR3 comprising (and in some embodiments consisting of) the amino acid sequence of any one of SEQ ID NOs: 105-117 and 170-174; or a variant thereof comprising up to about 5 (such as about any of 1, 2, 3, 4, or 5) amino acid substitutions in the LC-CDR regions.

In some embodiments, the anti-PMC construct comprises an antibody moiety that specifically binds to a complex comprising a PSA peptide and an MHC class I protein, wherein the antibody moiety comprises a) a heavy chain variable domain comprising (and in some embodiments consisting of) the amino acid sequence of any one of SEQ ID NOs: 14-26 and 145-149 or a variant thereof having at least about 95% (such as at least about any of 95%, 96%, 97%, 98%, or 99%) sequence identify to any one of SEQ ID NOs: 14-26 and 145-149; and b) a light chain variable domain comprising (and in some embodiments consisting of) the amino acid sequence of any one of SEQ ID NO: 27-39 and 150-154 or a variant thereof having at least about 95% (such as at least about any of 95%, 96%, 97%, 98%, or 99%) sequence identity to any one of SEQ ID NOs: 27-39 and 150-154. In some embodiments, the antibody moiety comprises a heavy chain variable domain comprising (and in some embodiments consisting of) the amino acid sequence of any one of SEQ ID NOs: 14-26 and 145-149 and a light chain variable domain comprising (and in some embodiments consisting of) the amino acid sequence of any one of SEQ ID NOs: 27-39 and 150-154.

In some embodiments, the anti-PMC construct comprises a first antibody moiety that competes for binding to a target PSA/MHC class I complex with a second antibody moiety according to any of the antibody moieties described above. In some embodiments, the first antibody moiety binds to the same, or substantially the same, epitope as the second antibody moiety. In some embodiments, binding of the first antibody moiety to the target PSA/MHC class I complex inhibits binding of the second antibody moiety to the target PSA/MHC class I complex by at least about 70% (such as by at least about any of 75%, 80%, 85%, 90%, 95%, 98% or 99%), or vice versa. In some embodiments, the first antibody moiety and the second antibody moiety cross-compete for binding to the target PSA/MHC class I complex, i.e., each of the first and second antibody moieties competes with the other for binding to the target PSA/MHC class I complex.

In some embodiments, according to any of the anti-PMC constructs described above, the isolated anti-PMC construct is a full-length antibody. In some embodiments, the isolated anti-PMC construct is monospecific. In some embodiments, the isolated anti-PMC construct is multi-specific. In some embodiments, the isolated anti-PMC construct is bispecific. In some embodiments, the isolated anti-PMC molecule is a tandem scFv, a diabody (Db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody. In some embodiments, the isolated anti-PMC construct is a tandem scFv comprising two scFvs linked by a peptide linker. In some embodiments, the peptide linker comprises (and in some embodiments consists of) the amino acid sequence GGGGS (SEQ ID NO: 175).

In some embodiments, according to any of the anti-PMC constructs described above, the anti-PMC construct comprises an antibody moiety that specifically binds to a complex comprising a PSA peptide and an MHC class I protein, wherein the isolated anti-PMC construct further comprises a second antibody moiety that specifically binds to a second antigen. In some embodiments, the second antigen is an antigen on the surface of a T cell. In some embodiments, the T cell is selected from the group consisting of a cytotoxic T cell, a helper T cell, and a natural killer T cell. In some embodiments, the second antigen is selected from the group consisting of CD3γ, CD3δ, CD3ε, CD3ζ, CD28, OX40, GITR, CD137, CD27, CD40L, and HVEM. In some embodiments, the second antigen is CD3ε, and the isolated anti-PMC construct is a tandem scFv comprising an N-terminal scFv specific for the PSA/MHC class I complex and a C-terminal scFv specific for CD3ε. In some embodiments, the second antigen is an antigen on the surface of a natural killer cell, a neutrophil, a monocyte, a macrophage, or a dendritic cell.

In some embodiments, according to any of the anti-PMC constructs (such as isolated anti-PMC constructs) described above, the anti-PMC construct comprises an antibody moiety that specifically binds to a complex comprising a PSA peptide and an MHC class I protein, wherein the isolated anti-PMC construct is a chimeric antigen receptor (CAR). In some embodiments, the chimeric antigen receptor comprises an extracellular domain comprising the antibody moiety, a transmembrane domain, and an intracellular signaling domain. In some embodiments, the intracellular signaling domain comprises a CD3ζ intracellular signaling sequence and a co-stimulatory signaling sequence. In some embodiments, the co-stimulatory signaling sequence is a CD28 or 4-1BB intracellular signaling sequence.

In some embodiments, according to any of the anti-PMC constructs (such as isolated anti-PMC constructs) described above, the anti-PMC construct comprises an antibody moiety that specifically binds to a complex comprising a PSA peptide and an MHC class I protein, wherein the isolated anti-PMC construct is an immunoconjugate comprising the antibody moiety and an effector molecule. In some embodiments, the effector molecule is a therapeutic agent selected from the group consisting of a drug, a toxin, a radioisotope, a protein, a peptide, and a nucleic acid. In some embodiments, the therapeutic agent is a drug or a toxin. In some embodiments, the effector molecule is a label.

In some embodiments, there is provided a nucleic acid encoding an anti-PMC construct, or polypeptide component thereof. In some embodiments, there is provided a vector comprising the nucleic acid. In some embodiments, there is provided a host cell expressing or associated with an anti-PMC construct, or polypeptide component thereof. In some embodiments, there is provided an effector cell expressing or associated with an anti-PMC construct. In some embodiments, the effector cell is a T cell. In yet other embodiments, there is provided a pharmaceutical composition comprising an anti-PMC construct (such as an isolated anti-PMC construct) according to any of the embodiments described above. In some embodiments, the pharmaceutical composition further comprises a cell (such as an effector cell) associated with the anti-PMC construct. In some embodiments, there is provided a pharmaceutical composition comprising a nucleic acid or vector according to any of the embodiments described above.

In some embodiments, there is provided a method of detecting a cell presenting a complex comprising a PSA peptide and an MHC class I protein on its surface, comprising contacting the cell with an anti-PMC construct (such as an isolated anti-PMC construct) according to any of the embodiments described above comprising a) an antibody moiety that specifically binds to a complex comprising the PSA peptide and the MHC class I protein and b) a label, and detecting the presence of the label on the cell.

In some embodiments, there is provided a method of treating an individual having a PSA-positive disease, comprising administering to the individual an effective amount of a pharmaceutical composition comprising an anti-PMC construct (such as an isolated anti-PMC construct) according to any of the embodiments described above. In some embodiments, the pharmaceutical composition further comprises a cell (such as an effector cell) associated with the anti-PMC construct. In some embodiments, there is provided a method of treating an individual having a PSA-positive disease, comprising administering to the individual an effective amount of an effector cell expressing any of the anti-PMC CARs described above. In some embodiments, the effector cell is a T cell. In some embodiments, the PSA-positive disease is cancer. In some embodiments, the cancer is prostate cancer, breast cancer, ovarian cancer, or lung cancer. In some embodiments, the cancer is prostate cancer. In some embodiments, the cancer is hormone-resistant prostate cancer.

In some embodiments, there is provided a method of diagnosing an individual having a PSA-positive disease, comprising: a) administering an effective amount of an isolated anti-PMC construct according to any of the embodiments described above to the individual; and b) determining the level of the label in the individual, wherein a level of the label above a threshold level indicates that the individual has the PSA-positive disease. In some embodiments, there is provided a method of diagnosing an individual having a PSA-positive disease, comprising: a) contacting a sample derived from the individual with an isolated anti-PMC construct according to any of the embodiments described above; and b) determining the number of cells bound with the isolated anti-PMC construct in the sample, wherein a value for the number of cells bound with the isolated anti-PMC construct above a threshold level indicates that the individual has the PSA-positive disease. In some embodiments, the PSA-positive disease is cancer. In some embodiments, the cancer is prostate cancer, breast cancer, ovarian cancer, or lung cancer. In some embodiments, the cancer is prostate cancer. In some embodiments, the cancer is hormone-resistant prostate cancer.

Also provided are methods of making any of the constructs described above, articles of manufacture, and kits that are suitable for the methods described herein.

### BRIEF DESCRIPTION OF THE DRAWINGS

FIG. 1 shows the size exclusion chromatography (SEC) chromatogram of PSA 146-154 peptide/HLA-A*02:01 complex following concentration by ultrafiltration. Properly folded peptide/MHC complex monomers: 212 mL; misfolded aggregates: 111 mL.

FIG. 2 shows the results of phage clone ELISA for specific binding of biotinylated PSA 146-154 peptide/HLA-A*02:01 versus biotinylated control peptide mixture (P19)/HLA-A*02:01.

FIG. 3 shows the results of phage clone FACS binding assays for binding of PSA 146-154 peptide-loaded T2 cells versus control peptide mixture (P19)-loaded T2 cells. 1: negative control phage, PSA 146-154 peptide-loaded T2 cells; 2: PSA-specific phage clone, T2 cells without peptide loading; 3: PSA-specific phage clone. P19 control peptide mixture-loaded T2 cells; 4: PSA-specific phage clone, PSA 146-154 peptide-loaded T2 cells.

FIG. 4 shows the results of phage clone FACS binding assays for binding of PSA 146-154 peptide-loaded T2 cells versus control peptide mixture (P19)-loaded T2 cells. 1: PSA-specific phage clone, T2 cells without peptide loading; 2: PSA-specific phage clone, P19 control peptide mixture-loaded T2 cells; 3: PSA-specific phage clone. PSA 146-154 peptide-loaded T2 cells.

FIG. 5 shows a schematic representation of a chimeric antigen receptor construct.

FIG. 6 shows the killing of cancer cell lines positive for HLA-A*02:01 and either positive or negative for PSA, mediated by T cells expressing one of a panel of anti-PSA146/MHC CARs. Mock indicates non-transduced T cells.

### DETAILED DESCRIPTION OF THE INVENTION

The present application provides isolated constructs (referred to herein as “anti-PMC constructs”) that comprise an antibody moiety (referred to herein as an “anti-PMC antibody moiety”) that specifically binds to a complex comprising a PSA peptide and an MHC class I protein (referred to herein as a “PSA/MHC class I complex.” or “PMC”). The anti-PMC constructs specifically recognize PSA/MHC class I complexes, such as MHC-presented PSA peptides on the surface of cells expressing PSA. Anti-PMC constructs may specifically bind to the N-terminal portion, the C-terminal portion or the middle portion of the PSA peptide in the complex, and/or cross-react with at least one complex comprising the PSA peptide and a different subtype of the MHC class I protein (e.g., the anti-PMC construct binds to both a PSA peptide/HLA-A*02:01 complex and a PSA peptide/HLA-A*02:02 complex). The anti-PMC constructs allow for specific targeting of PMC-presenting cells (e.g., cells presenting on their surface a PSA peptide bound to an MHC class I molecule), such as disease cells overexpressing PSA. When present in a chimeric antigen receptor (CAR) expressed by a T cell, the anti-PMC antibody moiety specifically redirected human T cells to kill PMC-presenting target cells, such as PMC-presenting cancer cells. This strategy provides a significant technical advantage over using antibodies directed against the PSA protein, which cannot specifically target PMC-presenting cells. Furthermore, when fused to a detectable moiety, the anti-PMC antibody moiety allows for diagnosis and prognosis of PSA-positive diseases or disorders with high sensitivity to changes in the number and distribution of PMC-presenting cells, a potentially more relevant measure of disease progression than circulating PSA levels.

Using phage display technology, we generated multiple monoclonal antibodies that are specific and high affinity against PSA 146-154 peptide/HLA-A*02:01 complex. Flow cytometry and T-cell mediated cytotoxicity assays demonstrated that the antibodies recognized PSA peptide-pulsed T2 cells in a PSA- and HLA-A*02:01-restricted manner. Antibodies against PSA peptides in the context of an HLA complex can be effective therapeutic agents for cancer indications characterized by PSA overexpression.

The present application thus provides constructs (such as isolated constructs) comprising an antibody moiety that specifically binds to a complex comprising a PSA peptide and an MHC class I protein. The construct can be, for example, a full-length anti-PMC antibody, a multi-specific anti-PMC molecule (such as a bispecific anti-PMC antibody), an anti-PMC chimeric antigen receptor (“CAR”), or an anti-PMC immunoconjugate.

In another aspect, there are provided nucleic acids encoding the anti-PMC constructs or the anti-PMC antibody moiety portion of the constructs.

In another aspect, there are provided compositions comprising an anti-PMC construct comprising an antibody moiety that specifically binds to a complex comprising a PSA-peptide and an MHC class I protein. The composition can be a pharmaceutical composition comprising an anti-PMC construct or an effector cell expressing or associated with the anti-PMC construct (for example a T cell expressing an anti-PMC CAR).

Also provided are methods of making and using the anti-PMC constructs (or cells expressing or associated with the anti-PMC constructs) for treatment or diagnostic purposes, as well as kits and articles of manufacture useful for such methods.

### Definitions

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the invention contemplate any one or more of these aspects of treatment.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

“Activation”, as used herein in relation to T cells, refers to the state of a T cell that has been sufficiently stimulated to induce detectable cellular proliferation. Activation can also be associated with induced cytokine production, and detectable effector functions.

The term “antibody moiety” includes full-length antibodies and antigen-binding fragments thereof. A full-length antibody comprises two heavy chains and two light chains. The variable regions of the light and heavy chains are responsible for antigen binding. The variables region in both chains generally contain three highly variable loops called the complementarity determining regions (CDRs) (light chain (LC) CDRs including LC-CDR1. LC-CDR2, and LC-CDR3, heavy chain (HC) CDRs including HC-CDR1, HC-CDR2, and HC-CDR3), CDR boundaries for the antibodies and antigen-binding fragments disclosed herein may be defined or identified by the conventions of Kabat. Chothia. or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain). IgG4 (γ4 heavy chain), IgA1 (α1 heavy chain), or IgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody moiety “competes” for binding to a target PMC with a second antibody moiety when the first antibody moiety inhibits target PMC binding of the second antibody moiety by at least about 50% (such as at least about any of 55%, 60%, 65%, 70%, 75%, 80%, 85%, 90%, 95%, 98% or 99%) in the presence of an equimolar concentration of the first antibody moiety, or vice versa. A high throughput process for “binning” antibodies based upon their cross-competition is described in PCT Publication No. WO 03/48731.

As use herein, the term “specifically binds” or “is specific for” refers to measurable and reproducible interactions, such as binding between a target and an antibody or antibody moiety, that is determinative of the presence of the target in the presence of a heterogeneous population of molecules, including biological molecules. For example, an antibody or antibody moiety that specifically binds to a target (which can be an epitope) is an antibody or antibody moiety that binds this target with greater affinity, avidity, more readily, and/or with greater duration than its bindings to other targets. In some embodiments, an antibody or antibody moiety that specifically binds to an antigen reacts with one or more antigenic determinants of the antigen (for example a PSA peptide/MHC class I protein complex) with a binding affinity that is at least about 10 times its binding affinity for other targets.

An “isolated” anti-PMC construct as used herein refers to an anti-PMC construct that (1) is not associated with proteins found in nature. (2) is free of other proteins from the same source, (3) is expressed by a cell from a different species, or, (4) does not occur in nature.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al. U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR DEFINITIONS

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

The term “chimeric antibodies” refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natl. Acad Sci. USA, 81:6851-6855 (1984)).

The term “semi-synthetic” in reference to an antibody or antibody moiety means that the antibody or antibody moiety has one or more naturally occurring sequences and one or more non-naturally occurring (i.e., synthetic) sequences.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv.” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113. Rosenburg and Moore eds., Springer-Verlag, New York. pp. 269-315 (1994).

The term “diabodies” refers to small antibody fragments prepared by constructing scFv fragments (see preceding paragraph) typically with short linkers (such as about 5 to about 10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta. Curr. Op. Struct. Biol. 2:593-596 (1992).

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2. ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

The terms “Fc receptor” or “FcR” are used to describe a receptor that binds to the Fc region of an antibody. In some embodiments, an FcR of this invention is one that binds an IgG antibody (a γ receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M, in Daëron, Annu. Rev. Immunol. 15:203-234 (1997)). The term includes allotypes, such as FcγRIIIA allotypes: FcγRIIIA-Phe 158. FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

The “CH1 domain” of a human IgG Fe region (also referred to as “C1” of “H1” domain) usually extends from about amino acid 118 to about amino acid 215 (EU numbering system).

“Hinge region” is generally defined as stretching from Glu216 to Pro230 of human IgG (Burton. Molec. Immunol. 22:161-206 (1985)). Hinge regions of other IgG isotypes may be aligned with the IgG1 sequence by placing the first and last cysteine residues forming inter-heavy chain S-S bonds in the same positions.

The “CH2 domain” of a human IgG Fe region (also referred to as “C2” of “H2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” or “H3” domain) comprises the stretch of residues C-terminal to a CH2 domain in an Fc region (i.e. from about amino acid residue 341 to the C-terminal end of an antibody sequence, typically at amino acid residue 446 or 447 of an IgG).

A “functional Fc fragment” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art.

An antibody with a variant IgG Fc with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent K_{d} or IC₅₀ value) than the parent polypeptide or a native sequence IgG Fc. According to some embodiments, the improvement in binding compared to a parent polypeptide is about 3 fold, such as about any of 5, 10, 25, 50, 60, 100, 150, 200, or up to 500 fold, or about 25% to 1000% improvement in binding. The polypeptide variant which “exhibits decreased binding” to an FcR, binds at least one FcR with lower affinity (e.g., higher apparent K_{d} or higher IC₅₀ value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The polypeptide comprising a variant Fc region which “exhibits increased ADCC” or mediates antibody-dependent cell-mediated cytotoxicity (ADCC) in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g. in an animal model etc. In some embodiments, the variant is from about 5 fold to about 100 fold, e.g. from about 25 to about 50 fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al. J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

An “effective amount” of an anti-PMC construct or composition as disclosed herein, is an amount sufficient to carry out a specifically stated purpose. An “effective amount” can be determined empirically and by known methods relating to the stated purpose.

The term “therapeutically effective amount” refers to an amount of an anti-PMC construct or composition as disclosed herein, effective to “treat” a disease or disorder in an individual. In the case of cancer, the therapeutically effective amount of the anti-PMC construct or composition as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the anti-PMC construct or composition as disclosed herein can prevent growth and/or kill existing cancer cells, it can be cytostatic and/or cytotoxic. In some embodiments, the therapeutically effective amount is a growth inhibitory amount. In some embodiments, the therapeutically effective amount is an amount that extends the survival of a patient. In some embodiments, the therapeutically effective amount is an amount that improves progression free survival of a patient.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The term “label” when used herein refers to a detectable compound or composition which can be conjugated directly or indirectly to the anti-PMC antibody moiety. The label may be detectable by itself (e.g., radioisotope labels or fluorescent labels) or, in the case of an enzymatic label, may catalyze chemical alteration of a substrate compound or composition which is detectable.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a.” “or,” and “the” include plural referents unless the context clearly dictates otherwise.

### Anti-PMC Constructs

In one aspect, the present invention provides PSA/MHC class I complex-specific constructs (anti-PMC constructs) that comprise an antibody moiety that specifically binds to a complex comprising a PSA peptide and an MHC class I protein (“PSA/MHC class I complex,” or “PMC”). The specificity of the anti-PMC construct derives from an anti-PMC antibody moiety, such as a full-length antibody or antigen-binding fragment thereof, that specifically binds to the PMC. In some embodiments, reference to a moiety (such as an antibody moiety) that specifically binds to a complex comprising a PSA peptide and an MHC class I protein means that the moiety binds to the PMC with a) an affinity that is at least about 10 (including for example at least about any of 10, 20, 30, 40, 50, 75, 100, 200, 300, 400, 500, 750, 1000 or more) times its binding affinity for each of full-length PSA, free PSA peptide, MHC class I protein not bound to a peptide, and MHC class I protein bound to a non-PSA peptide; or b) a K_{d} no more than about 1/10 (such as no more than about any of 1/10, 1/20, 1/30, 1/40, 1/50, 1/75, 1/100, 1/200, 1/300, 1/400, 1/500, 1/750, 1/1000 or less) times its K_{d} for binding to each of full-length PSA, free PSA peptide, MHC class I protein not bound to a peptide, and MHC class I protein bound to a non-PSA peptide. Binding affinity can be determined by methods known in the art, such as ELISA, fluorescence activated cell sorting (FACS) analysis, or radioimmunoprecipitation assay (RIA). K_{d} can be determined by methods known in the art, such as surface plasmon resonance (SPR) assay utilizing, for example. Biacore instruments, or kinetic exclusion assay (KinExA) utilizing, for example, Sapidyne instruments.

Contemplated anti-PMC constructs include, for example, full-length anti-PMC antibodies, multi-specific (such as bispecific) anti-PMC molecules, anti-PMC chimeric antigen receptors (CARs), and anti-PMC immunoconjugates.

For example, in some embodiments, there is provided an anti-PMC construct (such as an isolated anti-PMC construct) comprising an anti-PMC antibody moiety that specifically binds to a complex comprising a PSA peptide and an MHC class I protein. In some embodiments, the PSA peptide is PSA 146-154 (SEQ ID NO: 4). In some embodiments, the MHC class I protein is HLA-A02. In some embodiments, the MHC class I protein is HLA-A*02:01 (GenBank Accession No.: AAO20853). In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the anti-PMC construct binds the PMC with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the anti-PMC construct cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC construct cross-reacts with at least one (such as at least any of 2, 3, 4, or 5) complex comprising the PSA peptide and a different subtype of the MHC class I protein.

In some embodiments, there is provided an anti-PMC construct comprising an anti-PMC antibody moiety that specifically binds to a complex comprising a PSA 146-154 peptide (SEQ ID NO: 4) and HLA-A*02:01. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the anti-PMC construct binds the PMC with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the anti-PMC construct cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC construct cross-reacts with at least one (such as at least any of 2. 3, 4, or 5) complex comprising the PSA peptide and a different subtype of the MHC class I protein.

In some embodiments, there is provided an anti-PMC construct comprising an anti-PMC antibody moiety that specifically binds to a complex comprising a PSA peptide and an MHC class I protein, wherein the anti-PMC antibody moiety comprises: i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181; or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the anti-PMC construct binds the PMC with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the anti-PMC construct cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC construct cross-reacts with at least one (such as at least any of 2, 3, 4, or 5) complex comprising the PSA peptide and a different subtype of the MHC class I protein.

In some embodiments, there is provided an anti-PMC construct comprising an anti-PMC antibody moiety that specifically binds to a complex comprising a PSA peptide and an MHC class I protein, wherein the anti-PMC antibody moiety comprises: i) a heavy chain variable domain sequence comprising an HC-CDR1 comprising (and in some embodiments consisting of) the amino acid sequence of any one of SEQ ID NOs: 40-52, 155, and 156; or a variant thereof comprising up to about 5 (for example about any of 1, 2, 3, 4, or 5) amino acid substitutions; an HC-CDR2 comprising (and in some embodiments consisting of) the amino acid sequence of any one of SEQ ID NOs: 53-65 and 157; or a variant thereof comprising up to about 5 (for example about any of 1, 2, 3, 4, or 5) amino acid substitutions; and an HC-CDR3 comprising (and in some embodiments consisting of) the amino acid sequence of any one of SEQ ID NOs: 66-78 and 158-162; or a variant thereof comprising up to about 5 (for example about any of 1, 2, 3, 4, or 5) amino acid substitutions; and ii) a light chain variable domain sequence comprising an LC-CDR1 comprising (and in some embodiments consisting of) the amino acid sequence of any one of SEQ ID NOs: 79-91 and 163-166; or a variant thereof comprising up to about 5 (for example about any of 1, 2, 3, 4, or 5) amino acid substitutions; an LC-CDR2 comprising (and in some embodiments consisting of) the amino acid sequence of any one of SEQ ID NOs: 92-104 and 167-169; or a variant thereof comprising up to about 3 (for example about any of 1, 2, or 3) amino acid substitutions; and an LC-CDR3 comprising (and in some embodiments consisting of) the amino acid sequence of any one of SEQ ID NOs: 105-117 and 170-174; or a variant thereof comprising up to about 5 (for example about any of 1, 2, 3, 4, or 5) amino acid substitutions. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the anti-PMC construct binds the PMC with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the anti-PMC construct cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC construct cross-reacts with at least one (such as at least any of 2, 3, 4, or 5) complex comprising the PSA peptide and a different subtype of the MHC class I protein.

In some embodiments, there is provided an anti-PMC construct comprising an anti-PMC antibody moiety that specifically binds to a complex comprising a PSA peptide and an MHC class I protein, wherein the anti-PMC antibody moiety comprises: i) a heavy chain variable domain sequence comprising an HC-CDR1 comprising (and in some embodiments consisting of) the amino acid sequence of any one of SEQ ID NOs: 40-52, 155, and 156; an HC-CDR2 comprising (and in some embodiments consisting of) the amino acid sequence of any one of SEQ ID NOs: 53-65 and 157; and an HC-CDR3 comprising (and in some embodiments consisting of) the amino acid sequence of any one of SEQ ID NOs: 66-78 and 158-162; or a variant thereof comprising up to about 5 (for example about any of 1, 2, 3, 4, or 5) amino acid substitutions in the HC-CDR sequences; and ii) a light chain variable domain sequence comprising an LC-CDR1 comprising (and in some embodiments consisting of) the amino acid sequence of any one of SEQ ID NOs: 79-91 and 163-166; an LC-CDR2 comprising (and in some embodiments consisting of) the amino acid sequence of any one of SEQ ID NOs: 92-104 and 167-169; and an LC-CDR3 comprising (and in some embodiments consisting of) the amino acid sequence of any one of SEQ ID NOs: 105-117 and 170-174; or a variant thereof comprising up to about 5 (for example about any of 1, 2, 3, 4, or 5) amino acid substitutions in the LC-CDR sequences. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the anti-PMC construct binds the PMC with a K_{d} between about 0.1 pM to about 500 nM (such as about any of 0.1 pM, 1.0 pM, 10 pM, 50 pM, 100 pM, 500 pM, 1 nM. 10 nM, 50 nM, 100 nM, or 500 nM, including any ranges between these values). In some embodiments, the anti-PMC construct cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC construct cross-reacts with at least one (such as at least any of 2, 3, 4, or 5) complex comprising the PSA peptide and a different subtype of the MHC class I protein.

In some embodiments, there is provided an anti-PMC construct comprising an anti-PMC antibody moiety that specifically binds to a complex comprising a PSA peptide and an MHC class I protein, wherein the anti-PMC antibody moiety comprises a heavy chain variable domain comprising (and in some embodiments consisting of) the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising (and in some embodiments consisting of) the amino acid sequence of any one of SEQ ID NOs: 27-39 and 150-154, or a variant thereof having at least about 95% (for example at least about any of 96%, 97%, 98%, or 99%) sequence identity. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the anti-PMC construct binds the PMC with a K_{d} between about 0.1 pM to about 500 nM (such as about any of 0.1 pM, 1.0 pM, 10 pM, 50 pM, 100 pM, 500 pM, 1 nM, 10 nM, 5 nM, 50 nM, 100 nM, or 500 nM, including any ranges between these values). In some embodiments, the anti-PMC construct cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC construct cross-reacts with at least one (such as at least any of 2, 3, 4, or 5) complex comprising the PSA peptide and a different subtype of the MHC class I protein.

In some embodiments, there is provided an anti-PMC construct comprising a first anti-PMC antibody moiety that competes for binding to a target PSA/MHC class I complex with a second anti-PMC antibody moiety according to any of the anti-PMC antibody moieties described herein. In some embodiments, the first anti-PMC antibody moiety binds to the same, or substantially the same, epitope as the second anti-PMC antibody moiety. In some embodiments, binding of the first anti-PMC antibody moiety to the target PSA/MHC class I complex inhibits binding of the second anti-PMC antibody moiety to the target PSA/MHC class I complex by at least about 70% (such as by at least about any of 75%, 80%, 85%, 90%, 95%, 98% or 99%), or vice versa. In some embodiments, the first anti-PMC antibody moiety and the second anti-PMC antibody moiety cross-compete for binding to the target PSA/MHC class I complex, i.e., each of the first and second antibody moieties competes with the other for binding to the target PSA/MHC class I complex.

For example, in some embodiments, there is provided an anti-PMC construct comprising an anti-PMC antibody moiety that competes for binding to a target PSA/MHC class I complex with an antibody moiety comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181; or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions.

In some embodiments, there is provided an anti-PMC construct comprising an anti-PMC antibody moiety that competes for binding to a target PSA/MHC class I complex with an antibody moiety comprising i) a heavy chain variable domain sequence comprising an HC-CDR1 comprising (and in some embodiments consisting of) the amino acid sequence of any one of SEQ ID NOs: 40-52, 155, and 156; or a variant thereof comprising up to about 5 (for example about any of 1, 2, 3, 4, or 5) amino acid substitutions; an HC-CDR2 comprising (and in some embodiments consisting of) the amino acid sequence of any one of SEQ ID NOs: 53-65 and 157; or a variant thereof comprising up to about 5 (for example about any of 1, 2, 3, 4, or 5) amino acid substitutions; and an HC-CDR3 comprising (and in some embodiments consisting of) the amino acid sequence of any one of SEQ ID NOs: 66-78 and 158-162; or a variant thereof comprising up to about 5 (for example about any of 1, 2, 3, 4, or 5) amino acid substitutions; and ii) a light chain variable domain sequence comprising an LC-CDR1 comprising (and in some embodiments consisting of) the amino acid sequence of any one of SEQ ID NOs: 79-91 and 163-166; or a variant thereof comprising up to about 5 (for example about any of 1, 2, 3, 4, or 5) amino acid substitutions; an LC-CDR2 comprising (and in some embodiments consisting of) the amino acid sequence of any one of SEQ ID NOs: 92-104 and 167-169; or a variant thereof comprising up to about 3 (for example about any of 1, 2, or 3) amino acid substitutions; and an LC-CDR3 comprising (and in some embodiments consisting of) the amino acid sequence of any one of SEQ ID NOs: 105-117 and 170-174; or a variant thereof comprising up to about 5 (for example about any of 1, 2, 3, 4, or 5) amino acid substitutions.

In some embodiments, there is provided an anti-PMC construct comprising an anti-PMC antibody moiety that competes for binding to a target PSA/MHC class I complex with an antibody moiety comprising i) a heavy chain variable domain sequence comprising an HC-CDR1 comprising (and in some embodiments consisting of) the amino acid sequence of any one of SEQ ID NOs: 40-52, 155, and 156; an HC-CDR2 comprising (and in some embodiments consisting of) the amino acid sequence of any one of SEQ ID NOs: 53-65 and 157; and an HC-CDR3 comprising (and in some embodiments consisting of) the amino acid sequence of any one of SEQ ID NOs: 66-78 and 158-162; or a variant thereof comprising up to about 5 (for example about any of 1, 2, 3, 4, or 5) amino acid substitutions in the HC-CDR sequences; and ii) a light chain variable domain sequence comprising an LC-CDR1 comprising (and in some embodiments consisting of) the amino acid sequence of any one of SEQ ID NOs: 79-91 and 163-166; an LC-CDR2 comprising (and in some embodiments consisting of) the amino acid sequence of any one of SEQ ID NOs: 92-104 and 167-169; and an LC-CDR3 comprising (and in some embodiments consisting of) the amino acid sequence of any one of SEQ ID NOs: 105-117 and 170-174; or a variant thereof comprising up to about 5 (for example about any of 1, 2, 3, 4, or 5) amino acid substitutions in the LC-CDR sequences.

In some embodiments, there is provided an anti-PMC construct comprising an anti-PMC antibody moiety that competes for binding to a target PSA/MHC class I complex with an antibody moiety comprising a heavy chain variable domain comprising (and in some embodiments consisting of) the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising (and in some embodiments consisting of) the amino acid sequence of any one of SEQ ID NOs: 27-39 and 150-154, or a variant thereof having at least about 95% (for example at least about any of 96%, 97%, 98%, or 99%) sequence identity.

The different aspects are discussed in various sections below in further detail.

### Anti-PMC Antibody Moiety

The anti-PMC constructs comprise an anti-PMC antibody moiety that specifically binds to a complex comprising a PSA peptide and an MHC class I protein.

In some embodiments, the anti-PMC antibody moiety specifically binds to a PMC present on the surface of a cell. In some embodiments, the cell presents on its surface abnormally high levels of PSA. In some embodiments, the cell is a cancer cell. In some embodiments, the cancer cell is in a solid tumor. In some embodiments, the cancer cell is a metastatic cancer cell.

In some embodiments, the PSA peptide is an MHC class I-restricted peptide. In some embodiments, the PSA peptide is from about 8 to about 12 (such as about any of 8, 9, 10, 11, or 12) amino acids in length. In some embodiments, the PSA peptide is derived from human PSA (hPSA), mouse PSA (mPSA), or rat PSA (rPSA).

In some embodiments, the PSA peptide comprises (and in some embodiments consists of) the sequence of amino acids 108-117 of PSA (LTDAVKVMDL, SEQ ID NO: 3), amino acids 146-154 of PSA (KLQCVDLHV, SEQ ID NO: 4, also referred to herein as “PSA 146-154,” “PSA₁₄₆₋₁₅₄,” or “PSA146”), amino acids 154-163 of PSA (VISNDVCAQV, SEQ ID NO: 5), or amino acids 141-150 of PSA (FLTPKKLQCV, SEQ ID NO: 6).

In some embodiments, the MHC class I protein is HLA-A, HLA-B, HLA-C, HLA-E, HLA-F, or HLA-G. In some embodiments, the MHC class I protein is HLA-A. In some embodiments, the HLA-A is HLA-A02. In some embodiments, the HLA-A02 is HLA-A*02:01.

In some embodiments, the anti-PMC antibody moiety is a full-length antibody. In some embodiments, the anti-PMC antibody moiety is an antigen-binding fragment, for example an antigen-binding fragment selected from the group consisting of a Fab, a Fab′, a F(ab′)2, an Fv fragment, a disulfide stabilized Fv fragment (dsFv), and a single-chain antibody molecule (scFv). In some embodiments, the anti-PMC antibody moiety is an scFv. In some embodiments, the anti-PMC antibody moiety is human, humanized, or semi-synthetic.

In some embodiments, the anti-PMC antibody moiety specifically binds to the N-terminal portion of the PSA peptide in the complex. In some embodiments, the anti-PMC antibody moiety specifically binds to the N-terminal portion of the PSA peptide in the complex. In some embodiments, the anti-PMC antibody moiety specifically binds to the C-terminal portion of the PSA peptide in the complex. In some embodiments, the anti-PMC antibody moiety specifically binds to the middle portion of the PSA peptide in the complex.

In some embodiments, the anti-PMC antibody moiety (or the anti-PMC construct comprising the anti-PMC antibody moiety) binds to the complex comprising the PSA peptide and the MHC class I protein with an affinity that is at least about 10 (including for example at least about any of 10, 20, 30, 40, 50, 75, 100, 200, 300, 400, 500, 750, 1000 or more) times its binding affinity for each of full-length PSA, free PSA peptide, MHC class I protein not bound to a peptide, and MHC class I protein bound to a non-PSA peptide. In some embodiments, the anti-PMC antibody moiety (or the anti-PMC construct comprising the anti-PMC antibody moiety) binds to the complex comprising the PSA peptide and the MHC class I protein with a K_{d} no more than about 1/10 (such as no more than about any of 1/10, 1/20, 1/30, 1/40, 1/50, 1/75, 1/100, 1/200, 1/300, 1/400, 1/500, 1/750, 1/1000 or less) times its K_{d} for binding to each of full-length PSA, free PSA peptide, MHC class I protein not bound to a peptide, and MHC class I protein bound to a non-PSA peptide.

In some embodiments, the anti-PMC antibody moiety (or the anti-PMC construct comprising the anti-PMC antibody moiety) binds to the complex comprising the PSA peptide and the MHC class I protein with a K between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the anti-PMC antibody moiety (or the anti-PMC construct comprising the anti-PMC antibody moiety) binds to the complex comprising the PSA peptide and the MHC class I protein with a K_{d} between about 1 pM to about 250 pM (such as about any of 1, 10, 25, 50, 75, 100, 150, 200, or 250 pM, including any ranges between these values). In some embodiments, the anti-PMC antibody moiety (or the anti-PMC construct comprising the anti-PMC antibody moiety) binds to the complex comprising the PSA peptide and the MHC class I protein with a K_{d} between about 1 nM to about 500 nM (such as about any of 1, 10, 25, 50, 75, 100, 150, 200, 250, 300, 350, 400, 450, or 500 nM, including any ranges between these values).

In some embodiments, the anti-PMC antibody moiety specifically binds to a complex comprising a PSA peptide and an MHC class I protein, wherein the anti-PMC antibody moiety cross-reacts with at least one (such as at least any of 2, 3, 4, or 5) complex comprising the PSA peptide and an allelic variant of the MHC class I protein. In some embodiments, the allelic variant has up to about 10 (such as about any of 1, 2, 3, 4, 5, 6, 7, 8, 9, or 10) amino acid substitutions when compared to the MHC class I protein. In some embodiments, the allelic variant is the same serotype as the MHC class I protein. In some embodiments, the allelic variant is a different serotype than the MHC class I protein. In some embodiments, the anti-PMC antibody moiety does not cross-react with any complex comprising the PSA peptide and an allelic variant of the MHC class I protein. In some embodiments, the anti-PMC antibody moiety cross-reacts with at least one (such as at least any of 2, 3, 4, or 5) complex comprising the PSA peptide and a different subtype of the MHC class I protein.

In some embodiments, the anti-PMC antibody moiety specifically binds to a complex comprising a PSA peptide and an MHC class I protein, wherein the anti-PMC antibody moiety cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC antibody moiety does not cross-react with any complex comprising the MHC class I protein and a variant of the PSA peptide.

In some embodiments, the anti-PMC antibody moiety specifically binds to a complex comprising a PSA peptide and an MHC class I protein, wherein the anti-PMC antibody moiety cross-reacts with at least one complex comprising the MHC class I protein and an interspecies variant of the PSA peptide. In some embodiments, for example, the PSA peptide is human PSA peptide and the interspecies variant of the PSA peptide is a mouse or rat variant thereof. In some embodiments, the anti-PMC antibody moiety does not cross-react with a complex comprising the MHC class I protein and any interspecies variant of the PSA peptide.

For example, in some embodiments, the anti-PMC antibody moiety specifically binds to a complex comprising PSA 146-154 (SEQ ID NO: 4) and an MHC class I protein (such as HLA-A02, for example HLA-A*02:01). In some embodiments, the anti-PMC antibody moiety further binds to at least one (including at least about any of 2, 3, 4, 5, 6, or 7) of: a complex comprising an alanine-substituted PSA peptide of SEQ ID NO: 7 and an MHC class I protein (such as HLA-A02, for example HLA-A*02:01); a complex comprising an alanine-substituted PSA peptide of SEQ ID NO: 8 and an MHC class I protein (such as HLA-A02, for example HLA-A*02:01); a complex comprising an alanine-substituted PSA peptide of SEQ ID NO: 9 and an MHC class I protein (such as HLA-A02, for example HLA-A*02:01); a complex comprising an alanine-substituted PSA peptide of SEQ ID NO: 10 and an MHC class I protein (such as HLA-A02, for example HLA-A*02:01); a complex comprising an alanine-substituted PSA peptide of SEQ ID NO: 11 and an MHC class I protein (such as HLA-A02, for example HLA-A*02:01); a complex comprising an alanine-substituted PSA peptide of SEQ ID NO: 12 and an MHC class I protein (such as HLA-A02, for example HLA-A*02:01); and a complex comprising an alanine-substituted PSA peptide of SEQ ID NO: 13 and an MHC class I protein (such as HLA-A02, for example HLA-A*02:01).

In some embodiments, the anti-PMC antibody moiety specifically binds to a complex comprising PSA 146-154 (SEQ ID NO: 4) and HLA-A*02:01. In some embodiments, the anti-PMC antibody moiety further binds to at least one (including at least about any of 2, 3, 4, 5, 6, or 7) of: a complex comprising an alanine-substituted PSA peptide of SEQ ID NO: 7 and HLA-A*02:01; a complex comprising an alanine-substituted PSA peptide of SEQ ID NO: 8 and HLA-A*02:01; a complex comprising an alanine-substituted PSA peptide of SEQ ID NO: 9 and HLA-A*02:01; a complex comprising an alanine-substituted PSA peptide of SEQ ID NO: 10 and HLA-A*02:01; a complex comprising an alanine-substituted PSA peptide of SEQ ID NO: 11 and HLA-A*02:01; a complex comprising an alanine-substituted PSA peptide of SEQ ID NO: 12 and HLA-A*02:01; and a complex comprising an alanine-substituted PSA peptide of SEQ ID NO: 13 and HLA-A*02:01.

In some embodiments, the anti-PMC antibody moiety specifically binds to a complex comprising PSA 146-154 (SEQ ID NO: 4) and HLA-A*02:01. In some embodiments, the anti-PMC antibody moiety cross-reacts with at least one (including at least about any of 2, 3, 4, 5, or 6) of: a complex comprising PSA 146-154 (SEQ ID NO: 4) and HLA-A*02:02 (GenBank Accession No.: AFL91480), a complex comprising PSA 146-154 (SEQ ID NO: 4) and HLA-A*02:03 (GenBank Accession No.: AAA03604), a complex comprising PSA 146-154 (SEQ ID NO: 4) and HLA-A*02:05 (GenBank Accession No.: AAA03603), a complex comprising PSA 146-154 (SEQ ID NO: 4) and HLA-A*02:06 (GenBank Accession No.: CCB78868), a complex comprising PSA 146-154 (SEQ ID NO: 4) and HLA-A*02:07 (GenBank Accession No.: ACR55712), and a complex comprising PSA 146-154 (SEQ ID NO: 4) and HLA-A*02:11 (GenBank Accession No.: CAB56609).

In some embodiments, the anti-PMC antibody moiety is a semi-synthetic antibody moiety comprising fully human sequences and one or more synthetic regions. In some embodiments, the anti-PMC antibody moiety is a semi-synthetic antibody moiety comprising a fully human light chain variable domain and a semi-synthetic heavy chain variable domain comprising fully human FR1, HC-CDR1, FR2, HC-CDR2, FR3, and FR4 regions and a synthetic HC-CDR3. In some embodiments, the semi-synthetic heavy chain variable domain comprises a fully synthetic HC-CDR3 having a sequence from about 5 to about 25 (such as about any of 5, 6, 7, 8, 9, 10, 11, 12, 13, 14, 15, 16, 17, 18, 19, 20, 21, 22, 23, 24, or 25) amino acids in length. In some embodiments, the semi-synthetic heavy chain variable domain or the synthetic HC-CDR3 is obtained from a semi-synthetic library (such as a semi-synthetic human library) comprising fully synthetic HC-CDR3s having a sequence from about 5 to about 25 (such as about any of 5, 6, 7, 8, 9, 10, 11, 12, 13, 14, 15, 16, 17, 18, 19, 20, 21, 22, 23, 24, or 25) amino acids in length, wherein each amino acid in the sequence is randomly selected from the standard human amino acids, minus cysteine. In some embodiments, the synthetic HC-CDR3 is from about 7 to about 16 (such as about any of 7, 8, 9, 10, 11, 12, 13, 14, 15, or 16) amino acids in length.

The anti-PMC antibody moieties in some embodiments comprise specific sequences or certain variants of such sequences. In some embodiments, the amino acid substitutions in the variant sequences do not substantially reduce the ability of the anti-PMC antibody moiety to bind the PMC. For example, alterations that do not substantially reduce PMC binding affinity may be made. Alterations that substantially improve PMC binding affinity or affect some other property, such as specificity and/or cross-reactivity with related variants of the PMC, are also contemplated.

In some embodiments, the anti-PMC antibody moiety comprises i) a heavy chain variable domain comprising an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR3 comprising the amino acid sequence of SEQ ID NO: 122, 123, 124, 182, 183, or 184, or a variant thereof comprising up to about 3 (for example about any of 1, 2, or 3) amino acid substitutions.

In some embodiments, the anti-PMC antibody moiety comprises i) a heavy chain variable domain comprising an HC-CDR3 comprising the amino acid sequence of SEQ ID NO: 120 or 181; and ii) a light chain variable domain comprising an LC-CDR3 comprising the amino acid sequence of SEQ ID NO: 122, 123, 124, 182, 183, or 184.

In some embodiments, the anti-PMC antibody moiety comprises i) a heavy chain variable domain comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions.

In some embodiments, the anti-PMC antibody moiety comprises i) a heavy chain variable domain comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184.

In some embodiments, the anti-PMC antibody moiety comprises i) a heavy chain variable domain comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; or a variant thereof comprising up to about 3 (such as about any of 1, 2, or 3) amino acid substitutions in the HC-CDR sequences; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184; or a variant thereof comprising up to about 3 (such as about any of 1, 2, or 3) amino acid substitutions in the LC-CDR sequences.

In some embodiments, the anti-PMC antibody moiety comprises i) a heavy chain variable domain comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184. The sequences of the CDRs noted herein are provided in Table 2 below.

TABLE 2

| HC-CDR1 | SEQ ID | G-G/Y-S/T-F-S/T-S-Y-A/G |
|---|---|---|
| consensus | NO: 118 | |
| HC-CDR2 | SEQ ID | I-X-P-X-X-G-X-T |
| consensus | NO: 119 | |
| HC-CDR3 | SEQ ID | A-R-X-X-Y |
| consensus | NO: 120 | |
| 1 | | |
| HC-CDR3 | SEQ ID | A-R-Y-X-F/W/Y-D |
| consensus | NO: 180 | |
| 2 | | |
| LC-CDR1 | SEQ ID | S/T-S-N-F/I-G-A/N/S-G/N-Y |
| consensus | NO: 121 | |
| LC-CDR3 | SEQ ID | A/G-A/T-W-D/S-S-L-N/S-A/G-X-V |
| consensus | NO: 122 | |
| 1 | | |
| LC-CDR3 | SEQ ID | L-L-Y-X-G-G |
| consensus | NO: 123 | |
| 2 | | |
| LX-CDR3 | SEQ ID | Q-S-Y-D-S/T-S-L-S-G-X-V |
| consensus | NO: 124 | |
| 3 | | |
| LC-CDR3 | SEQ ID | A/G-X-W-D/E-X-S-L |
| consensus | NO: 182 | |
| 4 | | |
| LC-CDR3 | SEQ ID | L-L/V-F/Y-X-G-G |
| consensus | NO: 183 | |
| 5 | | |
| LC-CDR3 | SEQ ID | S/T-W/Y-X-S/T-S-L |
| consensus | NO: 184 | |
| 6 | | |

X can be any amino acid

In some embodiments, the anti-PMC antibody moiety comprises i) a heavy chain variable domain comprising an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions.

In some embodiments, the anti-PMC antibody moiety comprises i) a heavy chain variable domain comprising an HC-CDR3 comprising the amino acid sequence of any one of SEQ ID NOs: 66-78 and 158-162; and ii) a light chain variable domain comprising an LC-CDR3 comprising the amino acid sequence of any one of SEQ ID NOs: 105-117 and 170-174.

In some embodiments, the anti-PMC antibody moiety comprises i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions.

In some embodiments, the anti-PMC antibody moiety comprises i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174.

In some embodiments, the anti-PMC antibody moiety comprises i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences.

In some embodiments, the anti-PMC antibody moiety comprises i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions, wherein the amino acid substitutions are in HC-CDR1 or HC-CDR2; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions, wherein the amino acid substitutions are in HC-CDR1 or HC-CDR2.

In some embodiments, the anti-PMC antibody moiety comprises i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174. The sequences of the HC-CDRs noted herein are provided in Table 3 below and the LC-CDRs noted herein are provided in Table 4 below.

TABLE 3

| SEQ ID | GGTFSSYA | SEQ ID | IIPIPGIT | SEQ ID | ARSYKWGS |
|---|---|---|---|---|---|
| NO: 40 | | NO: 53 | | NO: 66 | SLVDA |
| HC-CDR1 | | HC-CDR2 | | HC-CDR3 | |
| 2 | | 2 | | 2 | |
| SEQ ID | GFTFSSYA | SEQ ID | ISGSGGST | SEQ ID | ARNYYSQY |
| NO: 41 | | NO: 54 | | NO: 67 | WMMDL |
| HC-CDR1 | | HC-CDR2 | | HC-CDR3 | |
| 3 | | 3 | | 3 | |
| SEQ ID | GYNFLNYG | SEQ ID | ISTYTGNT | SEQ ID | ARSSEYYT |
| NO: 42 | | NO: 55 | | NO: 68 | WDH |
| HC-CDR1 | | HC-CDR2 | | HC-CDR3 | |
| 5 | | 5 | | 5 | |
| SEQ ID | GYTFTGYY | SEQ ID | FDPEDGET | SEQ ID | ARYGFDY |
| NO: 43 | | NO: 56 | | NO: 69 | |
| HC-CDR1 | | HC-CDR2 | | HC-CDR3 | |
| 6 | | 6 | | 6 | |
| SEQ ID | GGSFSDYY | SEQ ID | INHSGGT | SEQ ID | ARYNEYGS |
| NO: 44 | | NO: 57 | | NO: 70 | GYDK |
| HC-CDR1 | | HC-CDR2 | | HC-CDR3 | |
| 8 | | 8 | | 8 | |
| SEQ ID | GYTFTSYG | SEQ ID | ISAYNGNT | SEQ ID | ARSSQYY |
| NO: 45 | | NO: 58 | | NO: 71 | VWDS |
| HC-CDR1 | | HC-CDR2 | | HC-CDR3 | |
| 9 | | 9 | | 9 | |
| SEQ ID | GGTFSSYA | SEQ ID | INPNSGGT | SEQ ID | ARWSYYYF |
| NO: 46 | | NO: 59 | | NO: 72 | QQFWSLDG |
| HC-CDR1 | | HC-CDR2 | | HC-CDR3 | |
| 10 | | 10 | | 10 | |
| SEQ ID | GYTFTSYG | SEQ ID | ISAYNGNT | SEQ ID | ARTNYNKY |
| NO: 47 | | NO: 60 | | NO: 73 | DI |
| HC-CDR1 | | HC-CDR2 | | HC-CDR3 | |
| 12 | | 12 | | 12 | |
| SEQ ID | GYTFTSYY | SEQ ID | FDPEDGET | SEQ ID | ARYSYDY |
| NO: 48 | | NO: 61 | | NO: 74 | |
| HC-CDR1 | | HC-CDR2 | | HC-CDR3 | |
| 13 | | 13 | | 13 | |
| SEQ ID | GYTFTGYF | SEQ ID | FDPEDGET | SEQ ID | ARYSYDL |
| NO: 49 | | NO: 62 | | NO: 75 | |
| HC-CDR1 | | HC-CDR2 | | HC-CDR3 | |
| 14 | | 14 | | 14 | |
| SEQ ID | GGTFSSYA | SEQ ID | IIPILGIA | SEQ ID | ARVSQPVY |
| NO: 50 | | NO: 63 | | NO: 76 | GSSTYDI |
| HC-CDR1 | | HC-CDR2 | | HC-CDR3 | |
| 15 | | 15 | | 15 | |
| SEQ ID | GYSFTSYW | SEQ ID | IYPGDSDT | SEQ ID | ARLVVPDA |
| NO: 51 | | NO: 64 | | NO: 77 | FDI |
| HC-CDR1 | | HC-CDR2 | | HC-CDR3 | |
| 17 | | 17 | | 17 | |
| SEQ ID | GYSFTSYW | SEQ ID | IYPGDSDT | SEQ ID | ARWGSRGF |
| NO: 52 | | NO: 65 | | NO: 78 | LDAFDI |
| HC-CDR1 | | HC-CDR2 | | HC-CDR3 | |
| 18 | | 18 | | 18 | |
| SEQ ID | GYSFTSYR | SEQ ID | IDPSDSYT | SEQ ID | ARWGLSWD |
| NO: 155 | | NO: 157 | | NO: 158 | GWGVTDY |
| HC-CDR1 | | HC-CDR2 | | HC-CDR3 | |
| 20 | | 22 | | 20 | |
| SEQ ID | GYTFTNYG | | | SEQ ID | ARYNYDT |
| NO: 156 | | | | NO: 159 | |
| HC-CDR1 | | | | HC-CDR3 | |
| 26 | | | | 21 | |
| | | | | SEQ ID | ARSFGAGY |
| | | | | NO: 160 | DS |
| | | | | HC-CDR3 | |
| | | | | 22 | |
| | | | | SEQ ID | ARYPWDH |
| | | | | NO: 161 | |
| | | | | HC-CDR3 | |
| | | | | 24 | |
| | | | | SEQ ID | ARSSYYGY |
| | | | | NO: 162 | LSDG |
| | | | | HC-CDR3 | |
| | | | | 26 | |

TABLE 4

| SEQ ID | NSNIGSNT | SEQ ID | SNN | SEQ ID | ATWDDSLNGPV |
|---|---|---|---|---|---|
| NO: 79 | | NO: 92 | | NO: 105 | |
| LC-CDR1 | | LC-CDR2 | | LC-CDR3 | |
| 2 | | 2 | | 2 | |
| SEQ ID | SSNFGAGYD | SEQ ID | GDT | SEQ ID | QSYDTSLSGSV |
| NO: 80 | | NO: 93 | | NO: 106 | |
| LC-CDR1 | | LC-CDR2 | | LC-CDR3 | |
| 3 | | 3 | | 3 | |
| SEQ ID | SSNIGAGYD | SEQ ID | GNS | SEQ ID | QSYDSSLSGWV |
| NO: 81 | | NO: 94 | | NO: 107 | |
| LC-CDR1 | | LC-CDR2 | | LC-CDR3 | |
| 5 | | 5 | | 5 | |
| SEQ ID | TGAVTSGYY | SEQ ID | TTG | SEQ ID | LLYSGGVWV |
| NO: 82 | | NO: 95 | | NO: 108 | |
| LC-CDR1 | | LC-CDR2 | | LC-CDR3 | |
| 6 | | 6 | | 6 | |
| SEQ ID | SYNIGNNY | SEQ ID | DNN | SEQ ID | GTWESSLSAYV |
| NO: 83 | | NO: 96 | | NO: 109 | |
| LC-CDR1 | | LC-CDR2 | | LC-CDR3 | |
| 8 | | 8 | | 8 | |
| SEQ ID | SSNFGAGYD | SEQ ID | GNS | SEQ ID | QSYDSSLSGWV |
| NO: 84 | | NO: 97 | | NO: 110 | |
| LC-CDR1 | | LC-CDR2 | | LC-CDR3 | |
| 9 | | 9 | | 9 | |
| SEQ ID | SSNIGSNT | SEQ ID | SNN | SEQ ID | AAWDDSLNGRWV |
| NO: 85 | | NO: 98 | | NO: 111 | |
| LC-CDR1 | | LC-CDR2 | | LC-CDR3 | |
| 10 | | 10 | | 10 | |
| SEQ ID | SSNIGAGYD | SEQ ID | GNS | SEQ ID | QSYDSSLSEV |
| NO: 86 | | NO: 99 | | NO: 112 | |
| LC-CDR1 | | LC-CDR2 | | LC-CDR3 | |
| 12 | | 12 | | 12 | |
| SEQ ID | TGAVTSGYY | SEQ ID | STS | SEQ ID | LLYYGGAYV |
| NO: 87 | | NO: 100 | | NO: 113 | |
| LC-CDR1 | | LC-CDR2 | | LC-CDR3 | |
| 13 | | 13 | | 13 | |
| SEQ ID | TGAVTSGYY | SEQ ID | STS | SEQ ID | LLYYGGAQWV |
| NO: 88 | | NO: 101 | | NO: 114 | |
| LC-CDR1 | | LC-CDR2 | | LC-CDR3 | |
| 14 | | 14 | | 14 | |
| SEQ ID | SSNLGSNS | SEQ ID | DNH | SEQ ID | AAWDDSLNSVV |
| NO: 89 | | NO: 102 | | NO: 115 | |
| LC-CDR1 | | LC-CDR2 | | LC-CDR3 | |
| 15 | | 15 | | 15 | |
| SEQ ID | SSNIGNNY | SEQ ID | DNY | SEQ ID | GTWDSSLSAGV |
| NO: 90 | | NO: 103 | | NO: 116 | |
| LC-CDR1 | | LC-CDR2 | | LC-CDR3 | |
| 17 | | 17 | | 17 | |
| SEQ ID | SSNIGNNY | SEQ ID | DND | SEQ ID | |
| NO: 91 | | NO: 104 | | NO: 117 | GTWDSSISSGV |
| LC-CDR1 | | LC-CDR2 | | LC-CDR3 | |
| 18 | | 18 | | 18 | |
| SEQ ID | QSISSY | SEQ ID | AAS | SEQ ID | QQSYSTPFT |
| NO: 163 | | NO: 167 | | NO: 170 | |
| LC-CDR1 | | LC-CDR2 | | LC-CDR3 | |
| 20 | | 20 | | 20 | |
| SEQ ID | TGTVTSTYY | SEQ ID | DVS | SEQ ID | LVFYGGVWV |
| NO: 164 | | NO: 168 | | NO: 171 | |
| LC-CDR1 | | LC-CDR2 | | LC-CDR3 | |
| 21 | | 22 | | 21 | |
| SEQ ID | SSDVGGYNY | SEQ ID | STN | SEQ ID | SSYTSSSRYV |
| NO: 165 | | NO: 169 | | NO: 172 | |
| LC-CDR1 | | LC-CDR2 | | LC-CDR3 | |
| 22 | | 24 | | 22 | |
| SEQ ID | SSNIGTNY | | | SEQ ID | LLYYGGQGV |
| NO: 166 | | | | NO: 173 | |
| LC-CDR1 | | | | LC-CDR3 | |
| 26 | | | | 24 | |
| | | | | SEQ ID | AAWDDSLSGLYV |
| | | | | NO: 174 | |
| | | | | LC-CDR3 | |
| | | | | 26 | |

In some embodiments, the anti-PMC antibody moiety comprises a heavy chain variable domain comprising the amino acid sequence of any one of SEQ ID NOs: 14-26 and 145-149, or a variant thereof having at least about 95% (including for example at least about any of 96%, 97%, 98%, or 99%) sequence identity, and a light chain variable domain comprising the amino acid sequence of any one of SEQ ID NOs: 27-39 and 150-154, or a variant thereof having at least about 95% (including for example at least any of 96%, 97%, 98%, or 99%) sequence identity.

In some embodiments, the anti-PMC antibody moiety comprises a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154.

The heavy and light chain variable domains can be combined in various pair-wise combinations to generate a number of anti-PMC antibody moieties.

For example, in some embodiments, the anti-PMC antibody moiety comprises a heavy chain variable domain comprising an HC-CDR1 comprising the amino acid sequence of SEQ ID NO: 40, or a variant thereof comprising up to about 5 (for example about any of 1, 2, 3, 4, or 5) amino acid substitutions; an HC-CDR2 comprising the amino acid sequence of SEQ ID NO:53, or a variant thereof comprising up to about 5 (for example about any of 1, 2. 3, 4, or 5) amino acid substitutions; and an HC-CDR3 comprising the amino acid sequence of SEQ ID NO: 66, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 79, or a variant thereof comprising up to about 5 (for example about any of 1, 2, 3, 4, or 5) amino acid substitutions; an LC-CDR2 comprising the amino acid sequence of SEQ ID NO: 92, or a variant thereof comprising up to about 3 (for example about any of 1, 2, or 3) amino acid substitutions; and an LC-CDR3 comprising the amino acid sequence of SEQ ID NO: 105, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 40, an HC-CDR2 comprising the amino acid sequence of SEQ ID NO:53, and an HC-CDR3 comprising the amino acid sequence of SEQ ID NO: 66, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 79, an LC-CDR2 comprising the amino acid sequence of SEQ ID NO: 92, and an LC-CDR3 comprising the amino acid sequence of SEQ ID NO: 105, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 40, an HC-CDR2 comprising the amino acid sequence of SEQ ID NO:53, and an HC-CDR3 comprising the amino acid sequence of SEQ ID NO: 66; and a light chain variable domain comprising an LC-CDR1 comprising the amino acid sequence of SEQ ID NO: 79, an LC-CDR2 comprising the amino acid sequence of SEQ ID NO: 92, and an LC-CDR3 comprising the amino acid sequence of SEQ ID NO: 105.

In some embodiments, the anti-PMC antibody moiety comprises a heavy chain variable domain comprising an HC-CDR1 comprising the amino acid sequence of SEQ ID NO: 41, or a variant thereof comprising up to about 5 (for example about any of 1, 2, 3, 4, or 5) amino acid substitutions; an HC-CDR2 comprising the amino acid sequence of SEQ ID NO:54, or a variant thereof comprising up to about 5 (for example about any of 1, 2, 3, 4, or 5) amino acid substitutions; and an HC-CDR3 comprising the amino acid sequence of SEQ ID NO: 67, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 80, or a variant thereof comprising up to about 5 (for example about any of 1, 2, 3, 4, or 5) amino acid substitutions; an LC-CDR2 comprising the amino acid sequence of SEQ ID NO: 93, or a variant thereof comprising up to about 3 (for example about any of 1, 2, or 3) amino acid substitutions; and an LC-CDR3 comprising the amino acid sequence of SEQ ID NO: 106, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 41, an HC-CDR2 comprising the amino acid sequence of SEQ ID NO:54, and an HC-CDR3 comprising the amino acid sequence of SEQ ID NO: 67, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 80, an LC-CDR2 comprising the amino acid sequence of SEQ ID NO: 93, and an LC-CDR3 comprising the amino acid sequence of SEQ ID NO: 106, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 41, an HC-CDR2 comprising the amino acid sequence of SEQ ID NO:54, and an HC-CDR3 comprising the amino acid sequence of SEQ ID NO: 67; and a light chain variable domain comprising an LC-CDR1 comprising the amino acid sequence of SEQ ID NO: 80, an LC-CDR2 comprising the amino acid sequence of SEQ ID NO: 93, and an LC-CDR3 comprising the amino acid sequence of SEQ ID NO: 106.

In some embodiments, the anti-PMC antibody moiety comprises a heavy chain variable domain comprising an HC-CDR1 comprising the amino acid sequence of SEQ ID NO:42, or a variant thereof comprising up to about 5 (for example about any of 1, 2, 3, 4, or 5) amino acid substitutions; an HC-CDR2 comprising the amino acid sequence of SEQ ID NO:55, or a variant thereof comprising up to about 5 (for example about any of 1, 2, 3, 4, or 5) amino acid substitutions; and an HC-CDR3 comprising the amino acid sequence of SEQ ID NO: 68, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 81, or a variant thereof comprising up to about 5 (for example about any of 1, 2, 3, 4, or 5) amino acid substitutions; an LC-CDR2 comprising the amino acid sequence of SEQ ID NO: 94, or a variant thereof comprising up to about 3 (for example about any of 1, 2, or 3) amino acid substitutions; and an LC-CDR3 comprising the amino acid sequence of SEQ ID NO: 107, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO:42, an HC-CDR2 comprising the amino acid sequence of SEQ ID NO:55, and an HC-CDR3 comprising the amino acid sequence of SEQ ID NO: 68, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 81, an LC-CDR2 comprising the amino acid sequence of SEQ ID NO: 94, and an LC-CDR3 comprising the amino acid sequence of SEQ ID NO: 107, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO:42, an HC-CDR2 comprising the amino acid sequence of SEQ ID NO:55, and an HC-CDR3 comprising the amino acid sequence of SEQ ID NO: 68; and a light chain variable domain comprising an LC-CDR1 comprising the amino acid sequence of SEQ ID NO: 81, an LC-CDR2 comprising the amino acid sequence of SEQ ID NO: 94, and an LC-CDR3 comprising the amino acid sequence of SEQ ID NO: 107.

In some embodiments, the anti-PMC antibody moiety comprises a heavy chain variable domain comprising an HC-CDR1 comprising the amino acid sequence of SEQ ID NO:43, or a variant thereof comprising up to about 5 (for example about any of 1, 2, 3, 4, or 5) amino acid substitutions; an HC-CDR2 comprising the amino acid sequence of SEQ ID NO:56, or a variant thereof comprising up to about 5 (for example about any of 1, 2, 3, 4, or 5) amino acid substitutions; and an HC-CDR3 comprising the amino acid sequence of SEQ ID NO: 69, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 82, or a variant thereof comprising up to about 5 (for example about any of 1, 2, 3, 4, or 5) amino acid substitutions; an LC-CDR2 comprising the amino acid sequence of SEQ ID NO: 95, or a variant thereof comprising up to about 3 (for example about any of 1, 2, or 3) amino acid substitutions; and an LC-CDR3 comprising the amino acid sequence of SEQ ID NO: 108, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO:43, an HC-CDR2 comprising the amino acid sequence of SEQ ID NO:56, and an HC-CDR3 comprising the amino acid sequence of SEQ ID NO: 69, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 82, an LC-CDR2 comprising the amino acid sequence of SEQ ID NO: 95, and an LC-CDR3 comprising the amino acid sequence of SEQ ID NO: 108, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO:43, an HC-CDR2 comprising the amino acid sequence of SEQ ID NO:56, and an HC-CDR3 comprising the amino acid sequence of SEQ ID NO: 69; and a light chain variable domain comprising an LC-CDR1 comprising the amino acid sequence of SEQ ID NO: 82, an LC-CDR2 comprising the amino acid sequence of SEQ ID NO: 95, and an LC-CDR3 comprising the amino acid sequence of SEQ ID NO: 108.

In some embodiments, the anti-PMC antibody moiety comprises a heavy chain variable domain comprising an HC-CDR1 comprising the amino acid sequence of SEQ ID NO:44, or a variant thereof comprising up to about 5 (for example about any of 1, 2, 3, 4, or 5) amino acid substitutions; an HC-CDR2 comprising the amino acid sequence of SEQ ID NO:57, or a variant thereof comprising up to about 5 (for example about any of 1, 2, 3, 4, or 5) amino acid substitutions; and an HC-CDR3 comprising the amino acid sequence of SEQ ID NO: 70, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 83, or a variant thereof comprising up to about 5 (for example about any of 1, 2, 3, 4, or 5) amino acid substitutions; an LC-CDR2 comprising the amino acid sequence of SEQ ID NO: 96, or a variant thereof comprising up to about 3 (for example about any of 1, 2, or 3) amino acid substitutions; and an LC-CDR3 comprising the amino acid sequence of SEQ ID NO: 109, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO:44, an HC-CDR2 comprising the amino acid sequence of SEQ ID NO:57, and an HC-CDR3 comprising the amino acid sequence of SEQ ID NO: 70, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 83, an LC-CDR2 comprising the amino acid sequence of SEQ ID NO: 96, and an LC-CDR3 comprising the amino acid sequence of SEQ ID NO: 109, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO:44, an HC-CDR2 comprising the amino acid sequence of SEQ ID NO:57, and an HC-CDR3 comprising the amino acid sequence of SEQ ID NO: 70; and a light chain variable domain comprising an LC-CDR1 comprising the amino acid sequence of SEQ ID NO: 83, an LC-CDR2 comprising the amino acid sequence of SEQ ID NO: 96, and an LC-CDR3 comprising the amino acid sequence of SEQ ID NO: 109.

In some embodiments, the anti-PMC antibody moiety comprises a heavy chain variable domain comprising an HC-CDR1 comprising the amino acid sequence of SEQ ID NO:45, or a variant thereof comprising up to about 5 (for example about any of 1, 2, 3, 4, or 5) amino acid substitutions; an HC-CDR2 comprising the amino acid sequence of SEQ ID NO:58, or a variant thereof comprising up to about 5 (for example about any of 1, 2, 3, 4, or 5) amino acid substitutions; and an HC-CDR3 comprising the amino acid sequence of SEQ ID NO: 71, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 84, or a variant thereof comprising up to about 5 (for example about any of 1, 2, 3, 4, or 5) amino acid substitutions; an LC-CDR2 comprising the amino acid sequence of SEQ ID NO: 97, or a variant thereof comprising up to about 3 (for example about any of 1, 2, or 3) amino acid substitutions; and an LC-CDR3 comprising the amino acid sequence of SEQ ID NO: 110, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO:45, an HC-CDR2 comprising the amino acid sequence of SEQ ID NO:58, and an HC-CDR3 comprising the amino acid sequence of SEQ ID NO: 71, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 84, an LC-CDR2 comprising the amino acid sequence of SEQ ID NO: 97, and an LC-CDR3 comprising the amino acid sequence of SEQ ID NO: 110, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO:45, an HC-CDR2 comprising the amino acid sequence of SEQ ID NO:58, and an HC-CDR3 comprising the amino acid sequence of SEQ ID NO: 71; and a light chain variable domain comprising an LC-CDR1 comprising the amino acid sequence of SEQ ID NO: 84, an LC-CDR2 comprising the amino acid sequence of SEQ ID NO: 97, and an LC-CDR3 comprising the amino acid sequence of SEQ ID NO: 110.

In some embodiments, the anti-PMC antibody moiety comprises a heavy chain variable domain comprising an HC-CDR1 comprising the amino acid sequence of SEQ ID NO:46, or a variant thereof comprising up to about 5 (for example about any of 1, 2, 3, 4, or 5) amino acid substitutions; an HC-CDR2 comprising the amino acid sequence of SEQ ID NO:59, or a variant thereof comprising up to about 5 (for example about any of 1, 2, 3, 4, or 5) amino acid substitutions; and an HC-CDR3 comprising the amino acid sequence of SEQ ID NO: 72, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 85, or a variant thereof comprising up to about 5 (for example about any of 1, 2, 3, 4, or 5) amino acid substitutions; an LC-CDR2 comprising the amino acid sequence of SEQ ID NO: 98, or a variant thereof comprising up to about 3 (for example about any of 1, 2, or 3) amino acid substitutions; and an LC-CDR3 comprising the amino acid sequence of SEQ ID NO: 111, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO:46, an HC-CDR2 comprising the amino acid sequence of SEQ ID NO:59, and an HC-CDR3 comprising the amino acid sequence of SEQ ID NO: 72, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 85, an LC-CDR2 comprising the amino acid sequence of SEQ ID NO: 98, and an LC-CDR3 comprising the amino acid sequence of SEQ ID NO: 111, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO:46, an HC-CDR2 comprising the amino acid sequence of SEQ ID NO:59, and an HC-CDR3 comprising the amino acid sequence of SEQ ID NO: 72; and a light chain variable domain comprising an LC-CDR1 comprising the amino acid sequence of SEQ ID NO: 85, an LC-CDR2 comprising the amino acid sequence of SEQ ID NO: 98, and an LC-CDR3 comprising the amino acid sequence of SEQ ID NO: 111.

In some embodiments, the anti-PMC antibody moiety comprises a heavy chain variable domain comprising an HC-CDR1 comprising the amino acid sequence of SEQ ID NO: 47, or a variant thereof comprising up to about 5 (for example about any of 1, 2, 3, 4, or 5) amino acid substitutions; an HC-CDR2 comprising the amino acid sequence of SEQ ID NO: 60, or a variant thereof comprising up to about 5 (for example about any of 1, 2, 3, 4, or 5) amino acid substitutions; and an HC-CDR3 comprising the amino acid sequence of SEQ ID NO: 73, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 86, or a variant thereof comprising up to about 5 (for example about any of 1, 2, 3, 4, or 5) amino acid substitutions; an LC-CDR2 comprising the amino acid sequence of SEQ ID NO: 99, or a variant thereof comprising up to about 3 (for example about any of 1, 2, or 3) amino acid substitutions; and an LC-CDR3 comprising the amino acid sequence of SEQ ID NO: 112, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 47, an HC-CDR2 comprising the amino acid sequence of SEQ ID NO: 60, and an HC-CDR3 comprising the amino acid sequence of SEQ ID NO: 73, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 86, an LC-CDR2 comprising the amino acid sequence of SEQ ID NO: 99, and an LC-CDR3 comprising the amino acid sequence of SEQ ID NO: 112, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 47, an HC-CDR2 comprising the amino acid sequence of SEQ ID NO: 60, and an HC-CDR3 comprising the amino acid sequence of SEQ ID NO: 73; and a light chain variable domain comprising an LC-CDR1 comprising the amino acid sequence of SEQ ID NO: 86, an LC-CDR2 comprising the amino acid sequence of SEQ ID NO: 99, and an LC-CDR3 comprising the amino acid sequence of SEQ ID NO: 112.

In some embodiments, the anti-PMC antibody moiety comprises a heavy chain variable domain comprising an HC-CDR1 comprising the amino acid sequence of SEQ ID NO: 48, or a variant thereof comprising up to about 5 (for example about any of 1, 2, 3, 4, or 5) amino acid substitutions; an HC-CDR2 comprising the amino acid sequence of SEQ ID NO: 61, or a variant thereof comprising up to about 5 (for example about any of 1, 2, 3, 4, or 5) amino acid substitutions; and an HC-CDR3 comprising the amino acid sequence of SEQ ID NO: 74, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 87, or a variant thereof comprising up to about 5 (for example about any of 1, 2, 3, 4, or 5) amino acid substitutions; an LC-CDR2 comprising the amino acid sequence of SEQ ID NO: 100, or a variant thereof comprising up to about 3 (for example about any of 1, 2, or 3) amino acid substitutions; and an LC-CDR3 comprising the amino acid sequence of SEQ ID NO: 113, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 48, an HC-CDR2 comprising the amino acid sequence of SEQ ID NO: 61, and an HC-CDR3 comprising the amino acid sequence of SEQ ID NO: 74, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 87, an LC-CDR2 comprising the amino acid sequence of SEQ ID NO: 100, and an LC-CDR3 comprising the amino acid sequence of SEQ ID NO: 113, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 48, an HC-CDR2 comprising the amino acid sequence of SEQ ID NO: 61, and an HC-CDR3 comprising the amino acid sequence of SEQ ID NO: 74; and a light chain variable domain comprising an LC-CDR1 comprising the amino acid sequence of SEQ ID NO: 87, an LC-CDR2 comprising the amino acid sequence of SEQ ID NO: 100, and an LC-CDR3 comprising the amino acid sequence of SEQ ID NO: 113.

In some embodiments, the anti-PMC antibody moiety comprises a heavy chain variable domain comprising an HC-CDR1 comprising the amino acid sequence of SEQ ID NO: 49, or a variant thereof comprising up to about 5 (for example about any of 1, 2, 3, 4, or 5) amino acid substitutions; an HC-CDR2 comprising the amino acid sequence of SEQ ID NO: 62, or a variant thereof comprising up to about 5 (for example about any of 1, 2, 3, 4, or 5) amino acid substitutions; and an HC-CDR3 comprising the amino acid sequence of SEQ ID NO: 75, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 88, or a variant thereof comprising up to about 5 (for example about any of 1, 2, 3, 4, or 5) amino acid substitutions; an LC-CDR2 comprising the amino acid sequence of SEQ ID NO: 101, or a variant thereof comprising up to about 3 (for example about any of 1, 2, or 3) amino acid substitutions; and an LC-CDR3 comprising the amino acid sequence of SEQ ID NO: 114, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 49, an HC-CDR2 comprising the amino acid sequence of SEQ ID NO: 62, and an HC-CDR3 comprising the amino acid sequence of SEQ ID NO: 75, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 88, an LC-CDR2 comprising the amino acid sequence of SEQ ID NO: 101, and an LC-CDR3 comprising the amino acid sequence of SEQ ID NO: 114, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 49, an HC-CDR2 comprising the amino acid sequence of SEQ ID NO: 62, and an HC-CDR3 comprising the amino acid sequence of SEQ ID NO: 75; and a light chain variable domain comprising an LC-CDR1 comprising the amino acid sequence of SEQ ID NO: 88, an LC-CDR2 comprising the amino acid sequence of SEQ ID NO: 101, and an LC-CDR3 comprising the amino acid sequence of SEQ ID NO: 114.

In some embodiments, the anti-PMC antibody moiety comprises a heavy chain variable domain comprising an HC-CDR1 comprising the amino acid sequence of SEQ ID NO: 50, or a variant thereof comprising up to about 5 (for example about any of 1, 2, 3, 4, or 5) amino acid substitutions; an HC-CDR2 comprising the amino acid sequence of SEQ ID NO: 63, or a variant thereof comprising up to about 5 (for example about any of 1, 2, 3, 4, or 5) amino acid substitutions; and an HC-CDR3 comprising the amino acid sequence of SEQ ID NO: 76, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 89, or a variant thereof comprising up to about 5 (for example about any of 1, 2, 3, 4, or 5) amino acid substitutions; an LC-CDR2 comprising the amino acid sequence of SEQ ID NO: 102, or a variant thereof comprising up to about 3 (for example about any of 1, 2, or 3) amino acid substitutions; and an LC-CDR3 comprising the amino acid sequence of SEQ ID NO: 115, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 50, an HC-CDR2 comprising the amino acid sequence of SEQ ID NO: 63, and an HC-CDR3 comprising the amino acid sequence of SEQ ID NO: 76, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 89, an LC-CDR2 comprising the amino acid sequence of SEQ ID NO: 102, and an LC-CDR3 comprising the amino acid sequence of SEQ ID NO: 115, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 50, an HC-CDR2 comprising the amino acid sequence of SEQ ID NO: 63, and an HC-CDR3 comprising the amino acid sequence of SEQ ID NO: 76; and a light chain variable domain comprising an LC-CDR1 comprising the amino acid sequence of SEQ ID NO: 89, an LC-CDR2 comprising the amino acid sequence of SEQ ID NO: 102, and an LC-CDR3 comprising the amino acid sequence of SEQ ID NO: 115.

In some embodiments, the anti-PMC antibody moiety comprises a heavy chain variable domain comprising an HC-CDR1 comprising the amino acid sequence of SEQ ID NO: 51, or a variant thereof comprising up to about 5 (for example about any of 1, 2, 3, 4, or 5) amino acid substitutions; an HC-CDR2 comprising the amino acid sequence of SEQ ID NO: 64, or a variant thereof comprising up to about 5 (for example about any of 1, 2, 3, 4, or 5) amino acid substitutions; and an HC-CDR3 comprising the amino acid sequence of SEQ ID NO: 77, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 90, or a variant thereof comprising up to about 5 (for example about any of 1, 2, 3, 4, or 5) amino acid substitutions; an LC-CDR2 comprising the amino acid sequence of SEQ ID NO: 103, or a variant thereof comprising up to about 3 (for example about any of 1, 2, or 3) amino acid substitutions; and an LC-CDR3 comprising the amino acid sequence of SEQ ID NO: 116, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 51, an HC-CDR2 comprising the amino acid sequence of SEQ ID NO: 64, and an HC-CDR3 comprising the amino acid sequence of SEQ ID NO: 77, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 90, an LC-CDR2 comprising the amino acid sequence of SEQ ID NO: 103, and an LC-CDR3 comprising the amino acid sequence of SEQ ID NO: 116, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 51, an HC-CDR2 comprising the amino acid sequence of SEQ ID NO: 64, and an HC-CDR3 comprising the amino acid sequence of SEQ ID NO: 77; and a light chain variable domain comprising an LC-CDR1 comprising the amino acid sequence of SEQ ID NO: 90, an LC-CDR2 comprising the amino acid sequence of SEQ ID NO: 103, and an LC-CDR3 comprising the amino acid sequence of SEQ ID NO: 116.

In some embodiments, the anti-PMC antibody moiety comprises a heavy chain variable domain comprising an HC-CDR1 comprising the amino acid sequence of SEQ ID NO: 52, or a variant thereof comprising up to about 5 (for example about any of 1, 2, 3, 4, or 5) amino acid substitutions; an HC-CDR2 comprising the amino acid sequence of SEQ ID NO: 65, or a variant thereof comprising up to about 5 (for example about any of 1, 2, 3, 4, or 5) amino acid substitutions; and an HC-CDR3 comprising the amino acid sequence of SEQ ID NO: 78, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 91, or a variant thereof comprising up to about 5 (for example about any of 1, 2, 3, 4, or 5) amino acid substitutions; an LC-CDR2 comprising the amino acid sequence of SEQ ID NO: 104, or a variant thereof comprising up to about 3 (for example about any of 1, 2, or 3) amino acid substitutions; and an LC-CDR3 comprising the amino acid sequence of SEQ ID NO: 117, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 52, an HC-CDR2 comprising the amino acid sequence of SEQ ID NO: 65, and an HC-CDR3 comprising the amino acid sequence of SEQ ID NO: 78, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 91, an LC-CDR2 comprising the amino acid sequence of SEQ ID NO: 104, and an LC-CDR3 comprising the amino acid sequence of SEQ ID NO: 117, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 52, an HC-CDR2 comprising the amino acid sequence of SEQ ID NO: 65, and an HC-CDR3 comprising the amino acid sequence of SEQ ID NO: 78; and a light chain variable domain comprising an LC-CDR1 comprising the amino acid sequence of SEQ ID NO: 91, an LC-CDR2 comprising the amino acid sequence of SEQ ID NO: 104, and an LC-CDR3 comprising the amino acid sequence of SEQ ID NO: 117.

In some embodiments, the anti-PMC antibody moiety comprises a heavy chain variable domain comprising an HC-CDR1 comprising the amino acid sequence of SEQ ID NO: 155, or a variant thereof comprising up to about 5 (for example about any of 1, 2, 3, 4, or 5) amino acid substitutions; an HC-CDR2 comprising the amino acid sequence of SEQ ID NO: 64, or a variant thereof comprising up to about 5 (for example about any of 1, 2, 3, 4, or 5) amino acid substitutions; and an HC-CDR3 comprising the amino acid sequence of SEQ ID NO: 158, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 163, or a variant thereof comprising up to about 5 (for example about any of 1, 2, 3, 4, or 5) amino acid substitutions; an LC-CDR2 comprising the amino acid sequence of SEQ ID NO: 167, or a variant thereof comprising up to about 3 (for example about any of 1, 2, or 3) amino acid substitutions; and an LC-CDR3 comprising the amino acid sequence of SEQ ID NO: 170, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 155, an HC-CDR2 comprising the amino acid sequence of SEQ ID NO: 64, and an HC-CDR3 comprising the amino acid sequence of SEQ ID NO: 158, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 163, an LC-CDR2 comprising the amino acid sequence of SEQ ID NO: 167, and an LC-CDR3 comprising the amino acid sequence of SEQ ID NO: 170, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 155, an HC-CDR2 comprising the amino acid sequence of SEQ ID NO: 64, and an HC-CDR3 comprising the amino acid sequence of SEQ ID NO: 158; and a light chain variable domain comprising an LC-CDR1 comprising the amino acid sequence of SEQ ID NO: 163, an LC-CDR2 comprising the amino acid sequence of SEQ ID NO: 167, and an LC-CDR3 comprising the amino acid sequence of SEQ ID NO: 170.

In some embodiments, the anti-PMC antibody moiety comprises a heavy chain variable domain comprising an HC-CDR1 comprising the amino acid sequence of SEQ ID NO: 48, or a variant thereof comprising up to about 5 (for example about any of 1, 2, 3, 4, or 5) amino acid substitutions; an HC-CDR2 comprising the amino acid sequence of SEQ ID NO: 56, or a variant thereof comprising up to about 5 (for example about any of 1, 2, 3, 4, or 5) amino acid substitutions; and an HC-CDR3 comprising the amino acid sequence of SEQ ID NO: 159, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 164, or a variant thereof comprising up to about 5 (for example about any of 1, 2, 3, 4, or 5) amino acid substitutions; an LC-CDR2 comprising the amino acid sequence of SEQ ID NO: 100, or a variant thereof comprising up to about 3 (for example about any of 1, 2, or 3) amino acid substitutions; and an LC-CDR3 comprising the amino acid sequence of SEQ ID NO: 171, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 48, an HC-CDR2 comprising the amino acid sequence of SEQ ID NO: 56, and an HC-CDR3 comprising the amino acid sequence of SEQ ID NO: 159, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 164, an LC-CDR2 comprising the amino acid sequence of SEQ ID NO: 100, and an LC-CDR3 comprising the amino acid sequence of SEQ ID NO: 171, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 48, an HC-CDR2 comprising the amino acid sequence of SEQ ID NO: 56, and an HC-CDR3 comprising the amino acid sequence of SEQ ID NO: 159; and a light chain variable domain comprising an LC-CDR1 comprising the amino acid sequence of SEQ ID NO: 164, an LC-CDR2 comprising the amino acid sequence of SEQ ID NO: 100, and an LC-CDR3 comprising the amino acid sequence of SEQ ID NO: 171.

In some embodiments, the anti-PMC antibody moiety comprises a heavy chain variable domain comprising an HC-CDR1 comprising the amino acid sequence of SEQ ID NO: 51, or a variant thereof comprising up to about 5 (for example about any of 1, 2, 3, 4, or 5) amino acid substitutions; an HC-CDR2 comprising the amino acid sequence of SEQ ID NO: 157, or a variant thereof comprising up to about 5 (for example about any of 1, 2, 3, 4, or 5) amino acid substitutions; and an HC-CDR3 comprising the amino acid sequence of SEQ ID NO: 160, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 165, or a variant thereof comprising up to about 5 (for example about any of 1, 2, 3, 4, or 5) amino acid substitutions; an LC-CDR2 comprising the amino acid sequence of SEQ ID NO: 168, or a variant thereof comprising up to about 3 (for example about any of 1, 2, or 3) amino acid substitutions; and an LC-CDR3 comprising the amino acid sequence of SEQ ID NO: 172, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 51, an HC-CDR2 comprising the amino acid sequence of SEQ ID NO: 157, and an HC-CDR3 comprising the amino acid sequence of SEQ ID NO: 160, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 165, an LC-CDR2 comprising the amino acid sequence of SEQ ID NO: 168, and an LC-CDR3 comprising the amino acid sequence of SEQ ID NO: 172, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 51, an HC-CDR2 comprising the amino acid sequence of SEQ ID NO: 157, and an HC-CDR3 comprising the amino acid sequence of SEQ ID NO: 160; and a light chain variable domain comprising an LC-CDR1 comprising the amino acid sequence of SEQ ID NO: 165, an LC-CDR2 comprising the amino acid sequence of SEQ ID NO: 168, and an LC-CDR3 comprising the amino acid sequence of SEQ ID NO: 172.

In some embodiments, the anti-PMC antibody moiety comprises a heavy chain variable domain comprising an HC-CDR1 comprising the amino acid sequence of SEQ ID NO: 48, or a variant thereof comprising up to about 5 (for example about any of 1, 2, 3, 4, or 5) amino acid substitutions; an HC-CDR2 comprising the amino acid sequence of SEQ ID NO: 56, or a variant thereof comprising up to about 5 (for example about any of 1, 2, 3, 4, or 5) amino acid substitutions; and an HC-CDR3 comprising the amino acid sequence of SEQ ID NO: 161, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 84, or a variant thereof comprising up to about 5 (for example about any of 1, 2, 3, 4, or 5) amino acid substitutions; an LC-CDR2 comprising the amino acid sequence of SEQ ID NO: 169, or a variant thereof comprising up to about 3 (for example about any of 1, 2, or 3) amino acid substitutions; and an LC-CDR3 comprising the amino acid sequence of SEQ ID NO: 173, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 48, an HC-CDR2 comprising the amino acid sequence of SEQ ID NO: 56, and an HC-CDR3 comprising the amino acid sequence of SEQ ID NO: 161, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 84, an LC-CDR2 comprising the amino acid sequence of SEQ ID NO: 169, and an LC-CDR3 comprising the amino acid sequence of SEQ ID NO: 173, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 48, an HC-CDR2 comprising the amino acid sequence of SEQ ID NO: 56, and an HC-CDR3 comprising the amino acid sequence of SEQ ID NO: 161; and a light chain variable domain comprising an LC-CDR1 comprising the amino acid sequence of SEQ ID NO: 84, an LC-CDR2 comprising the amino acid sequence of SEQ ID NO: 169, and an LC-CDR3 comprising the amino acid sequence of SEQ ID NO: 173.

In some embodiments, the anti-PMC antibody moiety comprises a heavy chain variable domain comprising an HC-CDR1 comprising the amino acid sequence of SEQ ID NO: 156, or a variant thereof comprising up to about 5 (for example about any of 1, 2, 3, 4, or 5) amino acid substitutions; an HC-CDR2 comprising the amino acid sequence of SEQ ID NO: 58, or a variant thereof comprising up to about 5 (for example about any of 1, 2, 3, 4, or 5) amino acid substitutions; and an HC-CDR3 comprising the amino acid sequence of SEQ ID NO: 162, or a variant thereof comprising up to about 5 (such as about any of 1, 2, 3, 4, or 5) amino acid substitutions; and a light chain variable domain comprising an LC-CDR1 comprising the amino acid sequence of SEQ ID NO: 166, or a variant thereof comprising up to about 5 (for example about any of 1, 2, 3, 4, or 5) amino acid substitutions; an LC-CDR2 comprising the amino acid sequence of SEQ ID NO: 92, or a variant thereof comprising up to about 3 (for example about any of 1, 2, or 3) amino acid substitutions; and an LC-CDR3 comprising the amino acid sequence of SEQ ID NO: 174, or a variant thereof comprising up to about 5 (such as about any of 1, 2, 3, 4, or 5) amino acid substitutions.

In some embodiments, the anti-PMC antibody moiety comprises a heavy chain variable domain comprising an HC-CDR1 comprising the amino acid sequence of SEQ ID NO: 156, an HC-CDR2 comprising the amino acid sequence of SEQ ID NO: 58, and an HC-CDR3 comprising the amino acid sequence of SEQ ID NO: 162, or a variant thereof comprising up to about 5 (such as about any of 1, 2, 3, 4, or 5) amino acid substitutions in the HC-CDR sequences; and a light chain variable domain comprising an LC-CDR1 comprising the amino acid sequence of SEQ ID NO: 166, an LC-CDR2 comprising the amino acid sequence of SEQ ID NO: 92, and an LC-CDR3 comprising the amino acid sequence of SEQ ID NO: 174, or a variant thereof comprising up to about 5 (such as about any of 1, 2, 3, 4, or 5) amino acid substitutions in the LC-CDR sequences.

In some embodiments, the anti-PMC antibody moiety comprises a heavy chain variable domain comprising an HC-CDR1 comprising the amino acid sequence of SEQ ID NO: 156, an HC-CDR2 comprising the amino acid sequence of SEQ ID NO: 58, and an HC-CDR3 comprising the amino acid sequence of SEQ ID NO: 162; and a light chain variable domain comprising an LC-CDR1 comprising the amino acid sequence of SEQ ID NO: 166, an LC-CDR2 comprising the amino acid sequence of SEQ ID NO: 92, and an LC-CDR3 comprising the amino acid sequence of SEQ ID NO: 174.

In some embodiments, the anti-PMC antibody moiety comprises a heavy chain variable domain comprising the amino acid sequence set forth in SEQ ID NO: 14, or a variant thereof having at least about 95% (for example at least about any of 96%, 97%, 98%, or 99%) sequence identity, and a light chain variable domain comprising the amino acid sequence set forth in SEQ ID NO: 27, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 14 and a light chain variable domain comprising the amino acid sequence set forth in SEQ ID NO: 27.

In some embodiments, the anti-PMC antibody moiety comprises a heavy chain variable domain comprising the amino acid sequence set forth in SEQ ID NO: 15, or a variant thereof having at least about 95% (including for example at least about any of 96%, 97%, 98%, or 99%) sequence identity, and a light chain variable domain comprising the amino acid sequence set forth in SEQ ID NO: 28, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 15 and a light chain variable domain comprising the amino acid sequence set forth in SEQ ID NO: 28.

In some embodiments, the anti-PMC antibody moiety comprises a heavy chain variable domain comprising the amino acid sequence set forth in SEQ ID NO: 16, or a variant thereof having at least about 95% (for example at least about any of 96%, 97%, 98%, or 99%) sequence identity, and a light chain variable domain comprising the amino acid sequence set forth in SEQ ID NO: 29, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 16 and a light chain variable domain comprising the amino acid sequence set forth in SEQ ID NO: 29.

In some embodiments, the anti-PMC antibody moiety comprises a heavy chain variable domain comprising the amino acid sequence set forth in SEQ ID NO: 17, or a variant thereof having at least about 95% (for example at least about any of 96%, 97%, 98%, or 99%) sequence identity, and a light chain variable domain comprising the amino acid sequence set forth in SEQ ID NO: 30, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 17 and a light chain variable domain comprising the amino acid sequence set forth in SEQ ID NO: 30.

In some embodiments, the anti-PMC antibody moiety comprises a heavy chain variable domain comprising the amino acid sequence set forth in SEQ ID NO: 18, or a variant thereof having at least about 95% (for example at least about any of 96%, 97%, 98%, or 99%) sequence identity, and a light chain variable domain comprising the amino acid sequence set forth in SEQ ID NO: 31, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 18 and a light chain variable domain comprising the amino acid sequence set forth in SEQ ID NO: 31.

In some embodiments, the anti-PMC antibody moiety comprises a heavy chain variable domain comprising the amino acid sequence set forth in SEQ ID NO: 19, or a variant thereof having at least about 95% (for example at least about any of 96%, 97%, 98%, or 99%) sequence identity, and a light chain variable domain comprising the amino acid sequence set forth in SEQ ID NO: 32, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 19 and a light chain variable domain comprising the amino acid sequence set forth in SEQ ID NO: 32.

In some embodiments, the anti-PMC antibody moiety comprises a heavy chain variable domain comprising the amino acid sequence set forth in SEQ ID NO: 20, or a variant thereof having at least about 95% (for example at least about any of 96%, 97%, 98%, or 99%) sequence identity, and a light chain variable domain comprising the amino acid sequence set forth in SEQ ID NO: 33, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 20 and a light chain variable domain comprising the amino acid sequence set forth in SEQ ID NO: 33.

In some embodiments, the anti-PMC antibody moiety comprises a heavy chain variable domain comprising the amino acid sequence set forth in SEQ ID NO: 21, or a variant thereof having at least about 95% (for example at least about any of 96%, 97%, 98%, or 99%) sequence identity, and a light chain variable domain comprising the amino acid sequence set forth in SEQ ID NO: 34, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 21 and a light chain variable domain comprising the amino acid sequence set forth in SEQ ID NO: 34.

In some embodiments, the anti-PMC antibody moiety comprises a heavy chain variable domain comprising the amino acid sequence set forth in SEQ ID NO: 22, or a variant thereof having at least about 95% (for example at least about any of 96%, 97%, 98%, or 99%) sequence identity, and a light chain variable domain comprising the amino acid sequence set forth in SEQ ID NO: 35, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 22 and a light chain variable domain comprising the amino acid sequence set forth in SEQ ID NO: 35.

In some embodiments, the anti-PMC antibody moiety comprises a heavy chain variable domain comprising the amino acid sequence set forth in SEQ ID NO: 23, or a variant thereof having at least about 95% (for example at least about any of 96%, 97%, 98%, or 99%) sequence identity, and a light chain variable domain comprising the amino acid sequence set forth in SEQ ID NO: 36, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 23 and a light chain variable domain comprising the amino acid sequence set forth in SEQ ID NO: 36.

In some embodiments, the anti-PMC antibody moiety comprises a heavy chain variable domain comprising the amino acid sequence set forth in SEQ ID NO: 24, or a variant thereof having at least about 95% (for example at least about any of 96%, 97%, 98%, or 99%) sequence identity, and a light chain variable domain comprising the amino acid sequence set forth in SEQ ID NO: 37, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 24 and a light chain variable domain comprising the amino acid sequence set forth in SEQ ID NO: 37.

In some embodiments, the anti-PMC antibody moiety comprises a heavy chain variable domain comprising the amino acid sequence set forth in SEQ ID NO: 25, or a variant thereof having at least about 95% (for example at least about any of 96%, 97%, 98%, or 99%) sequence identity, and a light chain variable domain comprising the amino acid sequence set forth in SEQ ID NO: 38, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 25 and a light chain variable domain comprising the amino acid sequence set forth in SEQ ID NO: 38.

In some embodiments, the anti-PMC antibody moiety comprises a heavy chain variable domain comprising the amino acid sequence set forth in SEQ ID NO: 26, or a variant thereof having at least about 95% (for example at least about any of 96%, 97%, 98%, or 99%) sequence identity, and a light chain variable domain comprising the amino acid sequence set forth in SEQ ID NO: 39, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 26 and a light chain variable domain comprising the amino acid sequence set forth in SEQ ID NO: 39.

In some embodiments, the anti-PMC antibody moiety comprises a heavy chain variable domain comprising the amino acid sequence set forth in SEQ ID NO: 145, or a variant thereof having at least about 95% (for example at least about any of 96%, 97%, 98%, or 99%) sequence identity, and a light chain variable domain comprising the amino acid sequence set forth in SEQ ID NO: 150, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 145 and a light chain variable domain comprising the amino acid sequence set forth in SEQ ID NO: 150.

In some embodiments, the anti-PMC antibody moiety comprises a heavy chain variable domain comprising the amino acid sequence set forth in SEQ ID NO: 146, or a variant thereof having at least about 95% (for example at least about any of 96%, 97%, 98%, or 99%) sequence identity, and a light chain variable domain comprising the amino acid sequence set forth in SEQ ID NO: 151, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 146 and a light chain variable domain comprising the amino acid sequence set forth in SEQ ID NO: 151.

In some embodiments, the anti-PMC antibody moiety comprises a heavy chain variable domain comprising the amino acid sequence set forth in SEQ ID NO: 147, or a variant thereof having at least about 95% (for example at least about any of 96%, 97%, 98%, or 99%) sequence identity, and a light chain variable domain comprising the amino acid sequence set forth in SEQ ID NO: 152, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 147 and a light chain variable domain comprising the amino acid sequence set forth in SEQ ID NO: 152.

In some embodiments, the anti-PMC antibody moiety comprises a heavy chain variable domain comprising the amino acid sequence set forth in SEQ ID NO: 148, or a variant thereof having at least about 95% (for example at least about any of 96%, 97%, 98%, or 99%) sequence identity, and a light chain variable domain comprising the amino acid sequence set forth in SEQ ID NO: 153, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 148 and a light chain variable domain comprising the amino acid sequence set forth in SEQ ID NO: 153.

In some embodiments, the anti-PMC antibody moiety comprises a heavy chain variable domain comprising the amino acid sequence set forth in SEQ ID NO: 149, or a variant thereof having at least about 95% (for example at least about any of 96%, 97%, 98%, or 99%) sequence identity, and a light chain variable domain comprising the amino acid sequence set forth in SEQ ID NO: 154, or a variant thereof having at least about 95% (including for example at least about any of 96%, 97%, 98%, or 99%) sequence identity. In some embodiments, the anti-PMC antibody moiety comprises a heavy chain variable domain comprising the amino acid sequence set forth in SEQ ID NO: 149 and a light chain variable domain comprising the amino acid sequence set forth in SEQ ID NO: 154.

In some embodiments, the anti-PMC antibody moiety competes for binding to a target PSA/MHC class I complex with a second anti-PMC antibody moiety according to any of the anti-PMC antibody moieties described herein. In some embodiments, the anti-PMC antibody moiety binds to the same, or substantially the same, epitope as the second anti-PMC antibody moiety. In some embodiments, binding of the anti-PMC antibody moiety to the target PSA/MHC class I complex inhibits binding of the second anti-PMC antibody moiety to the target PSA/MHC class I complex by at least about 70% (such as by at least about any of 75%, 80%, 85%, 90%, 95%, 98% or 99%), or vice versa. In some embodiments, the anti-PMC antibody moiety and the second anti-PMC antibody moiety cross-compete for binding to the target PSA/MHC class I complex, i.e., each of the antibody moieties competes with the other for binding to the target PSA/MHC class I complex.

For example, in some embodiments, the anti-PMC antibody moiety competes for binding to a target PSA/MHC class I complex with an antibody moiety comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181; or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions.

In some embodiments, the anti-PMC antibody moiety competes for binding to a target PSA/MHC class I complex with an antibody moiety comprising i) a heavy chain variable domain sequence comprising an HC-CDR1 comprising (and in some embodiments consisting of) the amino acid sequence of any one of SEQ ID NOs: 40-52, 155, and 156; or a variant thereof comprising up to about 5 (for example about any of 1, 2, 3, 4, or 5) amino acid substitutions; an HC-CDR2 comprising (and in some embodiments consisting of) the amino acid sequence of any one of SEQ ID NOs: 53-65 and 157; or a variant thereof comprising up to about 5 (for example about any of 1, 2, 3, 4, or 5) amino acid substitutions; and an HC-CDR3 comprising (and in some embodiments consisting of) the amino acid sequence of any one of SEQ ID NOs: 66-78 and 158-162; or a variant thereof comprising up to about 5 (for example about any of 1, 2, 3, 4, or 5) amino acid substitutions; and ii) a light chain variable domain sequence comprising an LC-CDR1 comprising (and in some embodiments consisting of) the amino acid sequence of any one of SEQ ID NOs: 79-91 and 163-166; or a variant thereof comprising up to about 5 (for example about any of 1, 2, 3, 4, or 5) amino acid substitutions; an LC-CDR2 comprising (and in some embodiments consisting of) the amino acid sequence of any one of SEQ ID NOs: 92-104 and 167-169; or a variant thereof comprising up to about 3 (for example about any of 1, 2, or 3) amino acid substitutions; and an LC-CDR3 comprising (and in some embodiments consisting of) the amino acid sequence of any one of SEQ ID NOs: 105-117 and 170-174; or a variant thereof comprising up to about 5 (for example about any of 1, 2, 3, 4, or 5) amino acid substitutions.

In some embodiments, the anti-PMC antibody moiety competes for binding to a target PSA/MHC class I complex with an antibody moiety comprising i) a heavy chain variable domain sequence comprising an HC-CDR1 comprising (and in some embodiments consisting of) the amino acid sequence of any one of SEQ ID NOs: 40-52, 155, and 156; an HC-CDR2 comprising (and in some embodiments consisting of) the amino acid sequence of any one of SEQ ID NOs: 53-65 and 157; and an HC-CDR3 comprising (and in some embodiments consisting of) the amino acid sequence of any one of SEQ ID NOs: 66-78 and 158-162; or a variant thereof comprising up to about 5 (for example about any of 1, 2, 3, 4, or 5) amino acid substitutions in the HC-CDR sequences; and ii) a light chain variable domain sequence comprising an LC-CDR1 comprising (and in some embodiments consisting of) the amino acid sequence of any one of SEQ ID NOs: 79-91 and 163-166; an LC-CDR2 comprising (and in some embodiments consisting of) the amino acid sequence of any one of SEQ ID NOs: 92-104 and 167-169; and an LC-CDR3 comprising (and in some embodiments consisting of) the amino acid sequence of any one of SEQ ID NOs: 105-117 and 170-174; or a variant thereof comprising up to about 5 (for example about any of 1, 2, 3, 4, or 5) amino acid substitutions in the LC-CDR sequences.

In some embodiments, the anti-PMC antibody moiety competes for binding to a target PSA/MHC class I complex with an antibody moiety comprising a heavy chain variable domain comprising (and in some embodiments consisting of) the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising (and in some embodiments consisting of) the amino acid sequence of any one of SEQ ID NOs: 27-39 and 150-154, or a variant thereof having at least about 95% (for example at least about any of 96%, 97%, 98%, or 99%) sequence identity.

### Full-Length Anti-PMC Antibodies

The anti-PMC constructs in some embodiments are full-length antibodies comprising an anti-PMC antibody moiety (also referred to herein as a “full-length anti-PMC antibody”). In some embodiments, the full-length antibody is a monoclonal antibody.

In some embodiments, the full-length anti-PMC antibody comprises an Fc sequence from an immunoglobulin, such as IgA, IgD, IgE, IgG, and IgM. In some embodiments, the full-length anti-PMC antibody comprises an Fc sequence of IgG, such as any of IgG1, IgG2, IgG3, or IgG4. In some embodiments, the full-length anti-PMC antibody comprises an Fc sequence of a human immunoglobulin. In some embodiments, the full-length anti-PMC antibody comprises an Fc sequence of a mouse immunoglobulin. In some embodiments, the full-length anti-PMC antibody comprises an Fc sequence that has been altered or otherwise changed so that it has enhanced antibody dependent cellular cytotoxicity (ADCC) or complement dependent cytotoxicity (CDC) effector function.

Thus, for example, in some embodiments, there is provided a full-length anti-PMC antibody comprising a) an anti-PMC antibody moiety that specifically binds to a complex comprising a PSA peptide and an MHC class I protein, and b) an Fc region. In some embodiments, the PSA peptide is PSA 146-154 (SEQ ID NO: 4). In some embodiments, the MHC class I protein is HLA-A02. In some embodiments, the MHC class I protein is HLA-A*02:01. In some embodiments, there is provided a full-length anti-PMC antibody comprising a) an anti-PMC antibody moiety that specifically binds to a complex comprising a PSA 146-154 peptide (SEQ ID NO: 4) and HLA-A*02:01, and b) an Fc region. In some embodiments, the Fc region comprises an IgG1 Fc sequence. In some embodiments, the Fc region comprises a human IgG1 Fc sequence. In some embodiments, the Fc region comprises a mouse IgG Fc sequence. In some embodiments, the anti-PMC antibody moiety cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC antibody moiety cross-reacts with at least one (such as at least any of 2, 3, 4, or 5) complex comprising the PSA peptide and a different subtype of the MHC class I protein.

In some embodiments, there is provided a full-length anti-PMC antibody comprising a) an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181; or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions, and b) an Fc region. In some embodiments, the Fc region comprises an IgG1 Fc sequence. In some embodiments, the Fc region comprises a human IgG1 Fc sequence. In some embodiments, the Fc region comprises a mouse IgG1 Fc sequence.

In some embodiments, there is provided a full-length anti-PMC antibody comprising a) an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184, and b) an Fc region. In some embodiments, the Fc region comprises an IgG1 Fc sequence. In some embodiments, the Fc region comprises a human IgG1 Fc sequence. In some embodiments, the Fc region comprises a mouse IgG1 Fc sequence.

In some embodiments, there is provided a full-length anti-PMC antibody comprising a) an anti-PMC antibody moiety that specifically binds to a complex comprising a PSA peptide and an MHC class I protein comprising i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions. In some embodiments, the Fc region comprises an IgG Fc sequence. In some embodiments, the Fc region comprises a human IgG1 Fc sequence. In some embodiments, the Fc region comprises a mouse IgG1 Fc sequence.

In some embodiments, there is provided a full-length anti-PMC antibody comprising a) an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences; and b) an Fc region. In some embodiments, the Fc region comprises an IgG1 Fc sequence. In some embodiments, the Fc region comprises a human IgG1 Fc sequence. In some embodiments, the Fc region comprises a mouse IgG1 Fc sequence.

In some embodiments, there is provided a full-length anti-PMC antibody comprising a) an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; and b) an Fc region. In some embodiments, the Fc region comprises an IgG1 Fc sequence. In some embodiments, the Fc region comprises a human IgG1 Fc sequence. In some embodiments, the Fc region comprises a mouse IgG1 Fc sequence.

In some embodiments, there is provided a full-length anti-PMC antibody comprising a) an anti-PMC antibody moiety that specifically binds to a complex comprising a PSA peptide and an MHC class I protein comprising a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% sequence identity; and b) an Fc region. In some embodiments, the Fc region comprises an IgG1 Fc sequence. In some embodiments, the Fc region comprises a human IgG1 Fc sequence. In some embodiments, the Fc region comprises a mouse IgG1 Fc sequence.

In some embodiments, there is provided a full-length anti-PMC antibody comprising a) an anti-PMC antibody moiety that specifically binds to a complex comprising a PSA peptide and an MHC class I protein comprising a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154; and b) an Fc region. In some embodiments, the Fc region comprises an IgG1 Fc sequence. In some embodiments, the Fc region comprises a human IgG1 Fc sequence. In some embodiments, the Fc region comprises a mouse IgG1 Fc sequence.

In some embodiments, the full-length anti-PMC antibody binds to a complex comprising a PSA peptide and an MHC class I protein with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the full-length anti-PMC antibody binds to a complex comprising a PSA peptide and an MHC class I protein with a K_{d} between about 1 pM to about 250 pM (such as about any of 1, 10, 25, 50, 75, 100, 150, 200, or 250 pM, including any ranges between these values).

### Multi-Specific Anti-PMC Molecules

The anti-PMC constructs in some embodiments comprise a multi-specific anti-PMC molecule comprising an anti-PMC antibody moiety and a second binding moiety (such as a second antigen-binding moiety). In some embodiments, the multi-specific anti-PMC molecule comprises an anti-PMC antibody moiety and a second antigen-binding moiety.

Multi-specific molecules are molecules that have binding specificities for at least two different antigens or epitopes (e.g., bispecific antibodies have binding specificities for two antigens or epitopes). Multi-specific molecules with more than two valencies and/or specificities are also contemplated. For example, trispecific antibodies can be prepared. Tutt et al. J. Immunol. 147: 60 (1991). It is to be appreciated that one of skill in the art could select appropriate features of individual multi-specific molecules described herein to combine with one another to form a multi-specific anti-PMC molecule of the invention.

Thus, for example, in some embodiments, there is provided a multi-specific (e.g., bispecific) anti-PMC molecule comprising a) an anti-PMC antibody moiety that specifically binds to a complex comprising a PSA peptide and an MHC class I protein, and b) a second binding moiety (such as an antigen-binding moiety). In some embodiments, the second binding moiety specifically binds to a complex comprising a different PSA peptide bound to the MHC class I protein. In some embodiments, the second scFv specifically binds to a complex comprising the PSA peptide bound to a different MHC class I protein. In some embodiments, the second binding moiety specifically binds to a different epitope on the complex comprising the PSA peptide and the MHC class I protein. In some embodiments, the second binding moiety specifically binds to a different antigen. In some embodiments, the second binding moiety specifically binds to an antigen on the surface of a cell, such as a cytotoxic cell. In some embodiments, the second binding moiety specifically binds to an antigen on the surface of a lymphocyte, such as a T cell, an NK cell, a neutrophil, a monocyte, a macrophage, or a dendritic cell. In some embodiments, the second binding moiety specifically binds to an effector T cell, such as a cytotoxic T cell (also known as cytotoxic T lymphocyte (CTL) or T killer cell).

In some embodiments, there is provided a multi-specific anti-PMC molecule comprising a) an anti-PMC antibody moiety that specifically binds to a complex comprising a PSA peptide and an MHC class I protein, and b) a second antigen-binding moiety that binds specifically to CD3. In some embodiments, the second antigen-binding moiety specifically binds to CD3ε. In some embodiments, the second antigen-binding moiety specifically binds to an agonistic epitope of CD3ε. The term “agonistic epitope”, as used herein, means (a) an epitope that, upon binding of the multi-specific molecule, optionally upon binding of several multi-specific molecules on the same cell, allows said multi-specific molecules to activate TCR signaling and induce T cell activation, and/or (b) an epitope that is solely composed of amino acid residues of the epsilon chain of CD3 and is accessible for binding by the multi-specific molecule, when presented in its natural context on T cells (i.e. surrounded by the TCR, the CD3γ chain, etc.), and/or (c) an epitope that, upon binding of the multi-specific molecule, does not lead to stabilization of the spatial position of CD3ϵ relative to CD3γ.

In some embodiments, there is provided a multi-specific anti-PMC molecule comprising a) an anti-PMC antibody moiety that specifically binds to a complex comprising a PSA peptide and an MHC class I protein, and b) a second antigen-binding moiety that binds specifically to an antigen on the surface of an effector cell, including for example CD3γ, CD3δ, CD3ε, CD3ζ, CD28, CD16a, CD56, CD68, and GDS2D.

In some embodiments, there is provided a multi-specific anti-PMC molecule comprising a) an anti-PMC antibody moiety that specifically binds to a complex comprising a PSA peptide and an MHC class I protein, and b) a second antigen-binding moiety that binds specifically to a component of the complement system, such as C1q, C1q is a subunit of the C enzyme complex that activates the serum complement system.

In some embodiments, the second antigen-binding moiety specifically binds to an Fc receptor. In some embodiments, the second antigen-binding moiety specifically binds to an Fcγ receptor (FcγR). The FcγR may be an FcγRIII present on the surface of natural killer (NK) cells or one of FcγRI, FcγRIIA, FcγRIIBI, FcγRIIB2, and FcγRIIIB present on the surface of macrophages, monocytes, neutrophils and/or dendritic cells. In some embodiments, the second antigen-binding moiety is an Fc region or functional fragment thereof. A “functional fragment” as used in this context refers to a fragment of an antibody Fc region that is still capable of binding to an FcR, in particular to an FcγR, with sufficient specificity and affinity to allow an FcγR bearing effector cell, in particular a macrophage, a monocyte, a neutrophil and/or a dendritic cell, to kill the target cell by cytotoxic lysis or phagocytosis. A functional Fc fragment is capable of competitively inhibiting the binding of the original, full-length Fc portion to an FcR such as the activating FcγRI. In some embodiments, a functional Fc fragment retains at least 30%, 40%, 50%, 60%, 70%, 80%, 90% or 95% of its affinity to an activating FcγR. In some embodiments, the Fc region or functional fragment thereof is an enhanced Fc region or functional fragment thereof. The term “enhanced Fc region”, as used herein, refers to an Fc region that is modified to enhance Fc receptor-mediated effector-functions, in particular antibody-dependent cell-mediated cytotoxicity (ADCC), complement-dependent cytotoxicity (CDC), and antibody-mediated phagocytosis. This can be achieved as known in the art, for example by altering the Fc region in a way that leads to an increased affinity for an activating receptor (e.g. FcγRIIIA (CD16A) expressed on natural killer (NK) cells) and/or a decreased binding to an inhibitory receptor (e.g. FcγRIIB1/B2 (CD32B)). In yet other embodiments, the second antigen-binding moiety is an antibody or antigen-binding fragment thereof that specifically binds to an FcR, in particular to an FcγR, with sufficient specificity and affinity to allow an FcγR bearing effector cell, in particular a macrophage, a monocyte, a neutrophil and/or a dendritic cell, to kill the target cell by cytotoxic lysis or phagocytosis.

In some embodiments, the multi-specific anti-PMC molecule allows killing of PMC-presenting target cells and/or can effectively redirect CTLs to lyse PMC-presenting target cells. In some embodiments, the multi-specific (e.g., bispecific) anti-PMC molecule of the present invention shows an in vitro EC₅₀ ranging from 10 to 500 ng/ml, and is able to induce redirected lysis of about 50% of the target cells through CTLs at a ratio of CTLs to target cells of from about 1:1 to about 50:1 (such as from about 1:1 to about 15:1, or from about 2:1 to about 10:1).

In some embodiments, the multi-specific (e.g., bispecific) anti-PMC molecule is capable of cross-linking a stimulated or unstimulated CTL and the target cell in such a way that the target cell is lysed. This offers the advantage that no generation of target-specific T cell clones or common antigen presentation by dendritic cells is required for the multi-specific anti-PMC molecule to exert its desired activity. In some embodiments, the multi-specific anti-PMC molecule of the present invention is capable of redirecting CTLs to lyse the target cells in the absence of other activating signals. In some embodiments, the second antigen-binding moiety of the multi-specific anti-PMC molecule specifically binds to CD3 (e.g., specifically binds to CD3ε), and signaling through CD28 and/or IL-2 is not required for redirecting CTLs to lyse the target cells.

Methods for measuring the preference of the multi-specific anti-PMC molecule to simultaneously bind to two antigens (e.g., antigens on two different cells) are within the normal capabilities of a person skilled in the art. For example, when the second binding moiety specifically binds to CD3, the multi-specific anti-PMC molecule may be contacted with a mixture of CD3⁺/PSA⁻ cells and CD3⁺/PSA⁺ cells. The number of multi-specific anti-PMC molecule-positive single cells and the number of cells cross-linked by multi-specific anti-PMC molecules may then be assessed by microscopy or fluorescence-activated cell sorting (FACS) as known in the art.

For example, in some embodiments, there is provided a multi-specific anti-PMC molecule comprising a) an anti-PMC antibody moiety that specifically binds to a complex comprising a PSA peptide and an MHC class I protein, and b) a second antigen-binding moiety. In some embodiments, the PSA peptide is PSA 146-154 (SEQ ID NO: 4). In some embodiments, the MHC class I protein is HLA-A02. In some embodiments, the MHC class I protein is HLA-A*02:01. In some embodiments, the second antigen-binding moiety specifically binds to a complex comprising a different PSA peptide bound to the MHC class I protein. In some embodiments, the second antigen-binding moiety specifically binds to a complex comprising the PSA peptide bound to a different MHC class I protein. In some embodiments, the second antigen-binding moiety specifically binds to a different epitope on the complex comprising the PSA peptide and the MHC class I protein. In some embodiments, the second antigen-binding moiety specifically binds to another antigen. In some embodiments, the second antigen-binding moiety specifically binds to an antigen on the surface of a cell, such as a PMC-presenting cell. In some embodiments, the second antigen-binding moiety specifically binds to an antigen on the surface of a cell that does not express PSA. In some embodiments, the second antigen-binding moiety specifically binds to an antigen on the surface of a cytotoxic cell. In some embodiments, the second antigen-binding moiety specifically binds to an antigen on the surface of a lymphocyte, such as a T cell, an NK cell, a neutrophil, a monocyte, a macrophage, or a dendritic cell. In some embodiments, the second antigen-binding moiety specifically binds to an antigen on the surface of an effector T cell, such as a cytotoxic T cell. In some embodiments, the second antigen-binding moiety specifically binds to an antigen on the surface of an effector cell, including for example CD3γ, CD3δ, CD3ε, CD3ζ, CD28, CD16a, CD56, CD68, and GDS2D. In some embodiments, the anti-PMC antibody moiety is human, humanized, or semi-synthetic. In some embodiments, the second antigen-binding moiety is an antibody moiety. In some embodiments, the second antigen-binding moiety is a human, humanized, or semi-synthetic antibody moiety. In some embodiments, the multi-specific anti-PMC molecule further comprises at least one (such as at least about any of 2, 3, 4, 5, or more) additional antigen-binding moieties.

In some embodiments, there is provided a multi-specific anti-PMC molecule comprising a) an anti-PMC antibody moiety that specifically binds to a complex comprising a PSA 146-154 peptide (SEQ ID NO: 4) and HLA-A*02:01, and b) a second antigen-binding moiety. In some embodiments, the anti-PMC antibody moiety cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC antibody moiety cross-reacts with at least one (such as at least any of 2, 3, 4, or 5) complex comprising the PSA peptide and a different subtype of the MHC class I protein.

In some embodiments, there is provided a multi-specific anti-PMC molecule comprising a) an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions, and b) a second antigen-binding moiety.

In some embodiments, there is provided a multi-specific anti-PMC molecule comprising a) an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184, and b) a second antigen-binding moiety.

In some embodiments, there is provided a multi-specific anti-PMC molecule comprising a) an anti-PMC antibody moiety that specifically binds to a complex comprising a PSA peptide and an MHC class I protein comprising i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions; and b) a second antigen-binding moiety.

In some embodiments, there is provided a multi-specific anti-PMC molecule comprising a) an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences; and b) a second antigen-binding moiety.

In some embodiments, there is provided a multi-specific anti-PMC molecule comprising a) an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; and b) a second antigen-binding moiety.

In some embodiments, there is provided a multi-specific anti-PMC molecule comprising a) an anti-PMC antibody moiety comprising a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% sequence identity; and b) a second scFv.

In some embodiments, there is provided a multi-specific anti-PMC molecule comprising a) an anti-PMC antibody moiety comprising a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154; and b) a second antigen-binding moiety.

In some embodiments, the multi-specific anti-PMC molecule is, for example, a diabody (Db), a single-chain diabody (scDb), a tandem scDb (Tandab), a linear dimeric scDb (LD-scDb), a circular dimeric scDb (CD-scDb), a di-diabody, a tandem scFv, a tandem di-scFv (e.g., a bispecific T cell engager), a tandem tri-scFv, a tri(a)body, a bispecific Fab2, a di-miniantibody, a tetrabody, an scFv-Fc-scFv fusion, a dual-affinity retargeting (DART) antibody, a dual variable domain (DVD) antibody, an IgG-scFab, an scFab-ds-scFv, an Fv2-Fc, an IgG-scFv fusion, a dock and lock (DNL) antibody, a knob-into-hole (KiH) antibody (bispecific IgG prepared by the KiH technology), a DuoBody (bispecific IgG prepared by the Duobody technology), a heteromultimeric antibody, or a heteroconjugate antibody. In some embodiments, the multi-specific anti-PMC molecule is a tandem scFv (e.g., a tandem di-scFv, such as a bispecific T cell engager).

Tandem scFv

The multi-specific anti-PMC molecule in some embodiments is a tandem scFv comprising a first scFv comprising an anti-PMC antibody moiety and a second scFv (also referred to herein as a “tandem scFv multi-specific anti-PMC antibody”). In some embodiments, the tandem scFv multi-specific anti-PMC antibody further comprises at least one (such as at least about any of 2, 3, 4, 5, or more) additional scFv.

In some embodiments, there is provided a tandem scFv multi-specific (e.g., bispecific) anti-PMC antibody comprising a) a first scFv that specifically binds to a complex comprising a PSA peptide and an MHC class I protein, and b) a second scFv. In some embodiments, the PSA peptide is PSA 146-154 (SEQ ID NO: 4). In some embodiments, the MHC class I protein is HLA-A02. In some embodiments, the MHC class I protein is HLA-A*02:01. In some embodiments, the second scFv specifically binds to a complex comprising a different PSA peptide bound to the MHC class I protein. In some embodiments, the second scFv specifically binds to a complex comprising the PSA peptide bound to a different MHC class I protein. In some embodiments, the second scFv specifically binds to a different epitope on the complex comprising the PSA peptide and the MHC class I protein. In some embodiments, the second scFv specifically binds to another antigen. In some embodiments, the second scFv specifically binds to an antigen on the surface of a cell, such as a PMC-presenting cell. In some embodiments, the second scFv specifically binds to an antigen on the surface of a cell that does not express PSA. In some embodiments, the second scFv specifically binds to an antigen on the surface of a cytotoxic cell. In some embodiments, the second scFv specifically binds to an antigen on the surface of a lymphocyte, such as a T cell, an NK cell, a neutrophil, a monocyte, a macrophage, or a dendritic cell. In some embodiments, the second scFv specifically binds to an antigen on the surface of an effector T cell, such as a cytotoxic T cell. In some embodiments, the second scFv specifically binds to an antigen on the surface of an effector cell, including for example CD3γ, CD3δ, CD3ε, CD3ζ, CD28, CD16a, CD56, CD68, and GDS2D. In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic. In some embodiments, the tandem scFv multi-specific anti-PMC antibody further comprises at least one (such as at least about any of 2, 3, 4, 5, or more) additional scFv.

In some embodiments, there is provided a tandem scFv multi-specific (e.g., bispecific) anti-PMC antibody comprising a) a first scFv that specifically binds to a complex comprising a PSA 146-154 peptide (SEQ ID NO: 4) and HLA-A*02:01, and b) a second scFv. In some embodiments, the first scFv cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the first scFv cross-reacts with at least one (such as at least any of 2, 3, 4, or 5) complex comprising the PSA peptide and a different subtype of the MHC class I protein.

In some embodiments, there is provided a tandem scFv multi-specific (e.g., bispecific) anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions, and b) a second scFv.

In some embodiments, there is provided a tandem scFv multi-specific (e.g., bispecific) anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184, and b) a second scFv.

In some embodiments, there is provided a tandem scFv multi-specific (e.g., bispecific) anti-PMC antibody comprising a) a first scFv that specifically binds to a complex comprising a PSA peptide and an MHC class I protein comprising i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions; and b) a second scFv.

In some embodiments, there is provided a tandem scFv multi-specific (e.g., bispecific) anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences; and b) a second scFv.

In some embodiments, there is provided a tandem scFv multi-specific (e.g., bispecific) anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; and b) a second scFv.

In some embodiments, there is provided a tandem scFv multi-specific (e.g., bispecific) anti-PMC antibody comprising a) a first scFv comprising a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% sequence identity; and b) a second scFv.

In some embodiments, there is provided a tandem scFv multi-specific (e.g., bispecific) anti-PMC antibody comprising a) a first scFv comprising a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154; and b) a second scFv.

In some embodiments, there is provided a tandem scFv multi-specific (e.g., bispecific) anti-PMC antibody comprising a) a first scFv that specifically binds to a complex comprising a PSA peptide and an MHC class I protein, and b) a second scFv, wherein the tandem scFv multi-specific anti-PMC antibody is a tandem di-scFv or a tandem tri-scFv. In some embodiments, the tandem scFv multi-specific anti-PMC antibody is a tandem di-scFv. In some embodiments, the tandem scFv multi-specific anti-PMC antibody is a bispecific T-cell engager.

For example, in some embodiments, there is provided a tandem di-scFv bispecific anti-PMC antibody comprising a) a first scFv that specifically binds to a complex comprising a PSA peptide and an MHC class I protein, and b) a second scFv that specifically binds to an antigen on the surface of a T cell. In some embodiments, the PSA peptide is PSA 146-154 (SEQ ID NO: 4). In some embodiments, the MHC class I protein is HLA-A02. In some embodiments, the MHC class I protein is HLA-A*02:01. In some embodiments, the second scFv specifically binds to an antigen on the surface of an effector T cell, such as a cytotoxic T cell. In some embodiments, the second scFv specifically binds to an antigen selected, for example, from the group consisting of CD3γ, CD3δ, CD3ε, CD3ζ, CD28, OX40, GITR. CD137, CD27, CD40L, and HVEM. In some embodiments, the second scFv specifically binds to an agonistic epitope on an antigen on the surface of a T cell, wherein the binding of the second scFv to the antigen enhances T cell activation. In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-PMC antibody comprising a) a first scFv that specifically binds to a complex comprising a PSA 146-154 peptide (SEQ ID NO: 4) and HLA-A*02:01, and b) a second scFv that specifically binds to an antigen on the surface of a T cell.

In some embodiments, there is provided a tandem di-scFv bispecific anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions, and b) a second scFv that specifically binds to an antigen on the surface of a T cell.

In some embodiments, there is provided a tandem di-scFv bispecific anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184, and b) a second scFv that specifically binds to an antigen on the surface of a T cell.

In some embodiments, there is provided a tandem di-scFv bispecific anti-PMC antibody comprising a) a first scFv that specifically binds to a complex comprising a PSA peptide and an MHC class I protein comprising i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions; and b) a second scFv that specifically binds to an antigen on the surface of a T cell.

In some embodiments, there is provided a tandem di-scFv bispecific anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences, and b) a second scFv that specifically binds to an antigen on the surface of a T cell.

In some embodiments, there is provided a tandem di-scFv bispecific anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; and b) a second scFv that specifically binds to an antigen on the surface of a T cell.

In some embodiments, there is provided a tandem di-scFv bispecific anti-PMC antibody comprising a) a first scFv comprising a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% (for example at least about any of 96%, 97%, 98%, or 99%) sequence identity, and b) a second scFv that specifically binds to an antigen on the surface of a T cell.

In some embodiments, there is provided a tandem di-scFv bispecific anti-PMC antibody comprising a) a first scFv comprising a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154, and b) a second scFv that specifically binds to an antigen on the surface of a T cell.

In some embodiments, there is provided a tandem di-scFv bispecific anti-PMC antibody comprising a) a first scFv that specifically binds to a complex comprising a PSA peptide and an MHC class I protein, and b) a second scFv that specifically binds to CD3ε. In some embodiments, the PSA peptide is PSA 146-154 (SEQ ID NO: 4). In some embodiments, the MHC class I protein is HLA-A02. In some embodiments, the MHC class I protein is HLA-A*02:01. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence GGGGS (SEQ ID NO: 175). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-PMC antibody comprising a) a first scFv that specifically binds to a complex comprising a PSA 146-154 peptide (SEQ ID NO: 4) and HLA-A*02:01, and b) a second scFv that specifically binds to CD3ε. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence GGGGS (SEQ ID NO: 175). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions, and b) a second scFv that specifically binds to CD3&. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence GGGGS (SEQ ID NO: 175). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184, and b) a second scFv that specifically binds to CD3ε. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence GGGGS (SEQ ID NO: 175). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-PMC antibody comprising a) a first scFv that specifically binds to a complex comprising a PSA peptide and an MHC class I protein comprising i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions, and b) a second scFv that specifically binds to CD3ε. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence GGGGS (SEQ ID NO: 175). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences, and b) a second scFv that specifically binds to CD3ε. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence GGGGS (SEQ ID NO: 175). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-PMC antibody comprising a) a first scFv that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; and b) a second scFv that specifically binds to CD3ε. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence GGGGS (SEQ ID NO: 175). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-PMC antibody comprising a) a first scFv comprising a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% (for example at least about any of 96%, 97%, 98%, or 99%) sequence identity, and b) a second scFv that specifically binds to CD3ε. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence GGGGS (SEQ ID NO: 175). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-PMC antibody comprising a) a first scFv comprising a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154, and b) a second scFv that specifically binds to CD3ε. In some embodiments, the first scFv is fused to the second scFv through linkage with a peptide linker. In some embodiments, the peptide linker is between about 5 to about 20 (such as about any of 5, 10. 15, or 20, including any ranges between these values) amino acids in length. In some embodiments, the peptide linker comprises (and in some embodiments consists of) the amino acid sequence GGGGS (SEQ ID NO: 175). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, the tandem di-scFv bispecific anti-PMC antibody binds to a complex comprising a PSA peptide and an MHC class I protein with a K_{d} between about 0.1 pM to about 500 nM (such as about any of 0.1 pM, 1.0 pM, 10 pM, 50 pM, 100 pM, 500 pM. 1 nM, 10 nM, 50 nM, 100 nM, or 500 nM, including any ranges between these values). In some embodiments, the tandem di-scFv bispecific anti-PMC antibody binds to a complex comprising a PSA peptide and an MHC class I protein with a K_{d} between about 1 nM to about 500 nM (such as about any of 1, 10, 25, 50, 75, 100, 150, 200, 250, 300, 350, 400, 450, or 500 nM, including any ranges between these values).

### Chimeric Antigen Receptor (CAR) and CAR Effector Cells

The anti-PMC construct in some embodiments is a chimeric antigen receptor (CAR) comprising an anti-PMC antibody moiety (also referred to herein as an “anti-PMC CAR”). Also provided is a CAR effector cell (e.g., T cell) comprising a CAR comprising an anti-PMC antibody moiety (also referred to herein as an “anti-PMC CAR effector cell”, e.g., “anti-PMC CAR T cell”).

The anti-PMC CAR comprises a) an extracellular domain comprising an anti-PMC antibody moiety that specifically binds to a complex comprising a PSA peptide and an MHC class I protein and b) an intracellular signaling domain. A transmembrane domain may be present between the extracellular domain and the intracellular domain.

Between the extracellular domain and the transmembrane domain of the anti-PMC CAR, or between the intracellular domain and the transmembrane domain of the anti-PMC CAR, there may be a spacer domain. The spacer domain can be any oligo- or polypeptide that functions to link the transmembrane domain to the extracellular domain or the intracellular domain in the polypeptide chain. A spacer domain may comprise up to about 300 amino acids, including for example about 10 to about 100, or about 25 to about 50 amino acids.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the α, β, δ, or γ chain of the T-cell receptor, CD28, CD3ε, CD3ζ, CD45, CD4, CD5, CD8, CD9. CD16, CD22, CD33, CD37, CD64, CD80, CD86, CD134, CD137, or CD154. In some embodiments, the transmembrane domain may be synthetic, in which case it may comprise predominantly hydrophobic residues such as leucine and valine. In some embodiments, a triplet of phenylalanine, tryptophan and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of the anti-PMC CAR. In some embodiments, the linker is a glycine-serine doublet.

In some embodiments, the transmembrane domain that naturally is associated with one of the sequences in the intracellular domain of the anti-PMC CAR is used (e.g., if an anti-PMC CAR intracellular domain comprises a CD28 co-stimulatory sequence, the transmembrane domain of the anti-PMC CAR is derived from the CD28 transmembrane domain). In some embodiments, the transmembrane domain can be selected or modified by amino acid substitution to avoid binding of such domains to the transmembrane domains of the same or different surface membrane proteins to minimize interactions with other members of the receptor complex.

The intracellular signaling domain of the anti-PMC CAR is responsible for activation of at least one of the normal effector functions of the immune cell in which the anti-PMC CAR has been placed in. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the anti-PMC CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or co-stimulatory signal (co-stimulatory signaling sequences).

Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs. The anti-PMC CAR constructs in some embodiments comprise one or more ITAMs.

Examples of ITAM containing primary signaling sequences that are of particular use in the invention include those derived from TCRζ, FcRγ, FcRβ, CD3γ, CD3δ, CD3ε, CD5, CD22, CD79a, CD79b, and CD66d.

In some embodiments, the anti-PMC CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the anti-PMC CAR of the invention. For example, the intracellular domain of the anti-PMC CAR can comprise a CD3ζ intracellular signaling sequence and a costimulatory signaling sequence. The costimulatory signaling sequence can be a portion of the intracellular domain of a costimulatory molecule including, for example, CD27, CD28, 4-1BB (CD137). OX40, CD30, CD40. PD-1, ICOS, lymphocyte function-associated antigen-1 (LFA-1), CD2, CD7, LIGHT, NKG2C, B7-H3, a ligand that specifically binds with CD83, and the like.

In some embodiments, the intracellular signaling domain of the anti-PMC CAR comprises the intracellular signaling sequence of CD3ζ and the intracellular signaling sequence of CD28. In some embodiments, the intracellular signaling domain of the anti-PMC CAR comprises the intracellular signaling sequence of CD3ζ and the intracellular signaling sequence of 4-1BB. In some embodiments, the intracellular signaling domain of the anti-PMC CAR comprises the intracellular signaling sequence of CD3ζ and the intracellular signaling sequences of CD28 and 4-1BB.

Thus, in some embodiments, there is provided an anti-PMC CAR comprising a) an extracellular domain comprising an anti-PMC antibody moiety that specifically binds to a complex comprising a PSA peptide and an MHC class I protein, b) a transmembrane domain, and c) an intracellular signaling domain. In some embodiments, the PSA peptide is PSA 146-154 (SEQ ID NO: 4). In some embodiments, the MHC class I protein is HLA-A02. In some embodiments, the MHC class I protein is HLA-A*02:01. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 or 4-1BB intracellular signaling sequence. In some embodiments, the intracellular domain comprises a CD3ζ intracellular signaling sequence and a CD28 or 4-1BB intracellular signaling sequence. In some embodiments, the anti-PMC CAR comprises the anti-PMC antibody moiety fused to the amino acid sequence of SEQ ID NO: 177. In some embodiments, the anti-PMC CAR comprises the anti-PMC antibody moiety fused to the amino acid sequence of SEQ ID NO: 178. In some embodiments, the anti-PMC antibody moiety is an scFv. In some embodiments, the scFv comprises heavy and light chain variable regions linked by a peptide linker, including, but not limited to, a peptide linker comprising the amino acid sequence of SEQ ID NO: 176.

In some embodiments, there is provided an anti-PMC CAR comprising a) an extracellular domain comprising an anti-PMC antibody moiety that specifically binds to a complex comprising a PSA 146-154 peptide (SEQ ID NO: 4) and HLA-A*02:01, b) a transmembrane domain, and c) an intracellular signaling domain. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 or 4-1BB intracellular signaling sequence. In some embodiments, the intracellular domain comprises a CD3ζ intracellular signaling sequence and a CD28 or 4-1BB intracellular signaling sequence. In some embodiments, the anti-PMC antibody moiety cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC antibody moiety cross-reacts with at least one (such as at least any of 2, 3, 4, or 5) complex comprising the PSA peptide and a different subtype of the MHC class I protein.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions, b) a transmembrane domain, and c) an intracellular signaling domain. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 or 4-1BB intracellular signaling sequence. In some embodiments, the intracellular domain comprises a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184; b) an intracellular signaling domain. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 or 4-1BB intracellular signaling sequence. In some embodiments, the intracellular domain comprises a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions; b) a transmembrane domain, and c) an intracellular signaling domain. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 or 4-1BB intracellular signaling sequence. In some embodiments, the intracellular domain comprises a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences; b) a transmembrane domain, and c) an intracellular signaling domain. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 or 4-1BB intracellular signaling sequence. In some embodiments, the intracellular domain comprises a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; b) an intracellular signaling domain. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 or 4-1BB intracellular signaling sequence. In some embodiments, the intracellular domain comprises a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% sequence identity; b) a transmembrane domain, and c) an intracellular signaling domain. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 or 4-1BB intracellular signaling sequence. In some embodiments, the intracellular domain comprises a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154; b) an intracellular signaling domain. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 or 4-1BB intracellular signaling sequence. In some embodiments, the intracellular domain comprises a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein, b) a transmembrane domain, and c) an intracellular signaling domain comprising a CD3ζ intracellular signaling sequence and a CD28 or 4-1BB intracellular signaling sequence. In some embodiments, the PSA peptide is PSA 146-154 (SEQ ID NO: 4). In some embodiments, the MHC class I protein is HLA-A02. In some embodiments, the MHC class I protein is HLA-A*02:01.

In some embodiments, there is provided an anti-PMC CAR comprising a) an extracellular domain comprising an anti-PMC antibody moiety that specifically binds to a complex comprising a PSA 146-154 peptide (SEQ ID NO: 4) and HLA-A*02:01, b) a transmembrane domain, and c) an intracellular signaling domain comprising a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions, b) a transmembrane domain, and c) an intracellular signaling domain comprising a CD3ζ intracellular signaling sequence and a CD28 or 4-1 BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184, b) a transmembrane domain, and c) an intracellular signaling domain comprising a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions; b) a transmembrane domain, and c) an intracellular signaling domain comprising a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences; b) a transmembrane domain, and c) an intracellular signaling domain comprising a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; b) a transmembrane domain, and c) an intracellular signaling domain comprising a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising i) a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% sequence identity; b) a transmembrane domain, and c) an intracellular signaling domain comprising a CD3ζ intracellular signaling sequence and a CD28 or 4-1BB intracellular signaling sequence.

In some embodiments, there is provided an anti-PMC CAR comprising a) an extracellular domain comprising an anti-PMC antibody moiety that specifically binds to a complex comprising a PSA peptide and an MHC class I protein comprising a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154; b) a transmembrane domain, and c) an intracellular signaling domain comprising a CD3ζ intracellular signaling sequence and a CD28 or 4-1BB intracellular signaling sequence.

Also provided herein are effector cells (such as lymphocytes, e.g., T cells) expressing an anti-PMC CAR.

Also provided is a method of producing an effector cell expressing an anti-PMC CAR, the method comprising introducing a vector comprising a nucleic acid encoding the anti-PMC CAR into the effector cell. In some embodiments, introducing the vector into the effector cell comprises transducing the effector cell with the vector. In some embodiments, introducing the vector into the effector cell comprises transfecting the effector cell with the vector. Transduction or transfection of the vector into the effector cell can be carried about using any method known in the art.

### Immunoconjugates

The anti-PMC constructs in some embodiments comprise an immunoconjugate comprising an anti-PMC antibody moiety attached to an effector molecule (also referred to herein as an “anti-PMC immunoconjugate”). In some embodiments the effector molecule is a therapeutic agent, such as a cancer therapeutic agent, which is either cytotoxic, cytostatic or otherwise provides some therapeutic benefit. In some embodiments, the effector molecule is a label, which can generate a detectable signal, either directly or indirectly.

In some embodiments, there is provided an anti-PMC immunoconjugate comprising an anti-PMC antibody moiety and a therapeutic agent (also referred to herein as an “antibody-drug conjugate”, or “ADC”). In some embodiments, the therapeutic agent is a toxin that is either cytotoxic, cytostatic or otherwise prevents or reduces the ability of the target cells to divide. The use of ADCs for the local delivery of cytotoxic or cytostatic agents, i.e., drugs to kill or inhibit tumor cells in the treatment of cancer (Syrigos and Epenetos. Anticancer Research 19:605-614 (1999); Niculescu-Duvaz and Springer, Adv. Drg. Del. Rev. 26:151-172 (1997); U.S. Pat. No. 4,975,278) allows targeted delivery of the drug moiety to target cells, and intracellular accumulation therein, where systemic administration of these unconjugated therapeutic agents may result in unacceptable levels of toxicity to normal cells as well as the target cells sought to be eliminated (Baldwin et al., Lancet (Mar. 15, 1986):603-605 (1986); Thorpe, (1985) “Antibody Carriers Of Cytotoxic Agents In Cancer Therapy: A Review,” in Monoclonal Antibodies '84: Biological And Clinical Applications. A. Pinchera et al. (eds.), pp. 475-506). Maximal efficacy with minimal toxicity is sought thereby. Importantly, since most normal cells do not present the PMC on their surface, they cannot bind the anti-PMC immunoconjugate, and are protected from the killing effect of the toxin or other therapeutic agents.

Therapeutic agents used in anti-PMC immunoconjugates include, for example, daunomycin, doxorubicin, methotrexate, and vindesine (Rowland et al., Cancer Immunol. Immunother. 21:183-187 (1986)). Toxins used in anti-PMC immunoconjugates include bacterial toxins such as diphtheria toxin, plant toxins such as ricin, small molecule toxins such as geldanamycin (Mandler et al., J. Nat. Cancer Inst. 92(19):1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Enzymatically active toxins and fragments thereof that can be used include, for example, diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct. 28, 1993.

Anti-PMC immunoconjugates of an anti-PMC antibody moiety and one or more small molecule toxins, such as a calicheamicin, maytansinoids, dolastatins, aurostatins, a trichothecene, and CC 1065, and the derivatives of these toxins that have toxin activity, are also contemplated herein.

In some embodiments, there is provided an anti-PMC immunoconjugate comprising a therapeutic agent that has an intracellular activity. In some embodiments, the anti-PMC immunoconjugate is internalized and the therapeutic agent is a cytotoxin that blocks the protein synthesis of the cell, therein leading to cell death. In some embodiments, the therapeutic agent is a cytotoxin comprising a polypeptide having ribosome-inactivating activity including, for example, gelonin, bouganin, saporin, ricin, ricin A chain, bryodin, diphtheria toxin, restrictocin, Pseudomonas exotoxin A and variants thereof. In some embodiments, where the therapeutic agent is a cytotoxin comprising a polypeptide having a ribosome-inactivating activity, the anti-PMC immunoconjugate must be internalized upon binding to the target cell in order for the protein to be cytotoxic to the cells.

In some embodiments, there is provided an anti-PMC immunoconjugate comprising a therapeutic agent that acts to disrupt DNA. In some embodiments, the therapeutic agent that acts to disrupt DNA is, for example, selected from the group consisting of enediyne (e.g., calicheamicin and esperamicin) and non-enediyne small molecule agents (e.g., bleomycin, methidiumpropyl-EDTA-Fe(II)). Other cancer therapeutic agents useful in accordance with the present application include, without limitation, daunorubicin, doxorubicin, distamycin A, cisplatin, mitomycin C. ecteinascidins, duocarmycin/CC-1065, and bleomycin/pepleomycin.

The present invention further contemplates an anti-PMC immunoconjugate formed between the anti-PMC antibody moiety and a compound with nucleolytic activity (e.g., a ribonuclease or a DNA endonuclease such as a deoxyribonuclease; DNase).

In some embodiments, the anti-PMC immunoconjugate comprises an agent that acts to disrupt tubulin. Such agents may include, for example, rhizoxin/maytansine, paclitaxel, vincristine and vinblastine, colchicine, auristatin dolastatin 10 MMAE, and peloruside A.

In some embodiments, the anti-PMC immunoconjugate comprises an alkylating agent including, for example, Asaley NSC 167780, AZQ NSC 182986, BCNU NSC 409962, Busulfan NSC 750, carboxyphthalatoplatinum NSC 271674, CBDCA NSC 241240. CCNU NSC 79037, CHIP NSC 256927, chlorambucil NSC 3088, chlorozotocin NSC 178248, cis-platinum NSC 119875, clomesone NSC 338947, cyanomorpholinodoxorubicin NSC 357704, cyclodisone NSC 348948, dianhydrogalactitol NSC 132313, fluorodopan NSC 73754, hepsulfam NSC 329680, hycanthone NSC 142982, melphalan NSC 8806, methyl CCNU NSC 95441, mitomycin C NSC 26980, mitozolamide NSC 353451, nitrogen mustard NSC 762. PCNU NSC 95466, piperazine NSC 344007, piperazinedione NSC 135758, pipobroman NSC 25154, porfiromycin NSC 56410, spirohydantoin mustard NSC 172112, teroxirone NSC 296934, tetraplatin NSC 363812, thio-tepa NSC 6396, triethylenemelamine NSC 9706, uracil nitrogen mustard NSC 34462, and Yoshi-864 NSC 102627.

In some embodiments, the cancer therapeutic agent portion of the anti-PMC immunoconjugate of the present application may comprise an antimitotic agent including, without limitation, allocolchicine NSC 406042, Halichondrin B NSC 609395, colchicine NSC 757, colchicine derivative NSC 33410, dolastatin 10 NSC 376128 (NG—auristatin derived), maytansine NSC 153858, rhizoxin NSC 332598, taxol NSC 125973, taxol derivative NSC 608832, thiocolchicine NSC 361792, trityl cysteine NSC 83265, vinblastine sulfate NSC 49842, and vincristine sulfate NSC 67574.

In some embodiments, the anti-PMC immunoconjugate comprises a topoisomerase I inhibitor including, without limitation, camptothecin NSC 94600, camptothecin, Na salt NSC 100880, aminocamptothecin NSC 603071, camptothecin derivative NSC 95382, camptothecin derivative NSC 107124, camptothecin derivative NSC 643833, camptothecin derivative NSC 629971, camptothecin derivative NSC 295500, camptothecin derivative NSC 249910, camptothecin derivative NSC 606985, camptothecin derivative NSC 374028, camptothecin derivative NSC 176323, camptothecin derivative NSC 295501, camptothecin derivative NSC 606172, camptothecin derivative NSC 606173, camptothecin derivative NSC 610458, camptothecin derivative NSC 618939, camptothecin derivative NSC 610457, camptothecin derivative NSC 610459, camptothecin derivative NSC 606499, camptothecin derivative NSC 610456, camptothecin derivative NSC 364830, camptothecin derivative NSC 606497, and morpholinodoxorubicin NSC 354646.

In some embodiments, the anti-PMC immunoconjugate comprises a topoisomerase II inhibitor including, without limitation, doxorubicin NSC 123127, amonafide NSC 308847, m-AMSA NSC 249992, anthrapyrazole derivative NSC 355644, pyrazoloacridine NSC 366140, bisantrene HCL NSC 337766, daunorubicin NSC 82151, deoxydoxorubicin NSC 267469, mitoxantrone NSC 301739, menogaril NSC 269148, N,N-dibenzyl daunomycin NSC 268242, oxanthrazole NSC 349174, rubidazone NSC 164011, VM-26 NSC 122819, and VP-16 NSC 141540.

In some embodiments, the anti-PMC immunoconjugate comprises an RNA or DNA antimetabolite including, without limitation, L-alanosine NSC 153353, 5-azacytidine NSC 102816, 5-fluorouracil NSC 19893, acivicin NSC 163501, aminopterin derivative NSC 132483, aminopterin derivative NSC 184692, aminopterin derivative NSC 134033, an antifol NSC 633713, an antifol NSC 623017, Baker's soluble antifol NSC 139105, dichlorallyl lawsone NSC 126771, brequinar NSC 368390, ftorafur (pro-drug) NSC 148958, 5,6-dihydro-5-azacytidine NSC 264880, methotrexate NSC 740, methotrexate derivative NSC 174121, N-(phosphonoacetyl)-L-aspartate (PALA) NSC 224131, pyrazofurin NSC 143095, trimetrexate NSC 352122, 3-HP NSC 95678, 2′-deoxy-5-fluorouridine NSC 27640, 5-HP NSC 107392, α-TGDR NSC 71851, aphidicolin glycinate NSC 303812, ara-C NSC 63878, 5-aza-2′-deoxycytidine NSC 127716, β-TGDR NSC 71261, cyclocytidine NSC 145668, guanazole NSC 1895, hydroxyurea NSC 32065, inosine glycodialdehyde NSC 118994, macbecin 11 NSC 330500, pyrazoloimidazole NSC 51143, thioguanine NSC 752, and thiopurine NSC 755.

In some embodiments, the anti-PMC immunoconjugate comprises a highly radioactive atom. A variety of radioactive isotopes are available for the production of radioconjugated antibodies. Examples include ²¹¹At, ¹³¹I, ¹²⁵Y, ¹⁸⁶Re, ¹⁸⁸Re, ¹⁵³Sm, ²¹²Bi, ³²P, ²¹²Pb and radioactive isotopes of Lu.

In some embodiments, the anti-PMC antibody moiety can be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

In some embodiments, an anti-PMC immunoconjugate may comprise an anti-PMC antibody moiety conjugated to a prodrug-activating enzyme. In some such embodiments, a prodrug-activating enzyme converts a prodrug (e.g., a peptidyl chemotherapeutic agent, see WO 81/01145) to an active drug, such as an anti-cancer drug. Such anti-PMC immunoconjugates are useful, in some embodiments, in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug. 5-fluorouracil; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibody moieties by recombinant DNA techniques well known in the art. See, e.g., Neuberger et al., Nature 312:604-608 (1984).

In some embodiments, the therapeutic portion of the anti-PMC immunoconjugates may be a nucleic acid. Nucleic acids that may be used include, but are not limited to, anti-sense RNA, genes or other polynucleotides, including nucleic acid analogs such as thioguanine and thiopurine.

The present application further provides anti-PMC immunoconjugates comprising an anti-PMC antibody moiety attached to an effector molecule, wherein the effector molecule is a label, which can generate a detectable signal, indirectly or directly. These anti-PMC immunoconjugates can be used for research or diagnostic applications, such as for the in vivo detection of cancer. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the label is a radioactive atom for scintigraphic studies, for example ⁹⁹Tc or ¹²³I, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

In some embodiments, the anti-PMC immunoconjugate is detectable indirectly. For example, a secondary antibody that is specific for the anti-PMC immunoconjugate and contains a detectable label can be used to detect the anti-PMC immunoconjugate.

Thus, for example, in some embodiments, there is provided an anti-PMC immunoconjugate comprising a) an anti-PMC antibody moiety that specifically binds to a complex comprising a PSA peptide and an MHC class I protein, and b) an effector molecule. In some embodiments, the PSA peptide is PSA 146-154 (SEQ ID NO: 4). In some embodiments, the MHC class I protein is HLA-A02. In some embodiments, the MHC class I protein is HLA-A*02:01. In some embodiments, the effector molecule is covalently attached to the anti-PMC antibody moiety. In some embodiments, the effector molecule is a therapeutic agent selected, for example, from the group consisting of a drug, a toxin, a radioisotope, a protein, a peptide, and a nucleic acid. In some embodiments, the effector molecular is a cancer therapeutic agent. In some embodiments, the cancer therapeutic agent is a chemotherapeutic. In some embodiments, the cancer therapeutic agent is a highly radioactive atom selected, for example, from the group consisting of ²¹³At, ¹³¹I, ¹²⁵I, ⁹⁰Y, ¹⁸⁶Re, ¹⁸⁸Re, ¹⁵³Sm, ²¹²Bi, ³²P, and ²¹²Pb. In some embodiments, the effector molecule is a label that can generate a detectable signal, either directly or indirectly. In some embodiments, the label is a radioisotope selected, for example, from the group consisting of ³H, ¹⁴C, ³²P, ³⁵S, ¹²³I, ¹²⁵I, and ¹³¹I. In some embodiments, the anti-PMC antibody moiety is an scFv. In some embodiments, the anti-PMC antibody moiety is human, humanized, or semi-synthetic.

In some embodiments, there is provided an anti-PMC immunoconjugate comprising a) an anti-PMC antibody moiety that specifically binds to a complex comprising a PSA 146-154 peptide (SEQ ID NO: 4) and HLA-A*02:01, and b) an effector molecule. In some embodiments, the effector molecule is covalently attached to the anti-PMC antibody moiety. In some embodiments, the effector molecule is a therapeutic agent selected, for example, from the group consisting of a drug, a toxin, a radioisotope, a protein, a peptide, and a nucleic acid. In some embodiments, the effector molecular is a cancer therapeutic agent. In some embodiments, the cancer therapeutic agent is a chemotherapeutic. In some embodiments, the cancer therapeutic agent is a highly radioactive atom selected, for example, from the group consisting of ²¹¹At, ¹³¹I, ¹²⁵I, ⁹⁰Y, ¹⁸⁶Re, ¹⁸⁸Re, ¹⁵³Sm, ²¹²Bi, ³²P, and ²¹²Pb. In some embodiments, the effector molecule is a label that can generate a detectable signal, either directly or indirectly. In some embodiments, the label is a radioisotope selected, for example, from the group consisting of ³H, ¹⁴C, ³²P, ³⁵S, ¹²³I, ¹²⁵I, and ¹³¹I. In some embodiments, the anti-PMC antibody moiety is an scFv. In some embodiments, the anti-PMC antibody moiety is human, humanized, or semi-synthetic. In some embodiments, the anti-PMC antibody moiety cross-reacts with at least one (such as at least any of 2, 3, 4, 5, or 6) complex comprising the MHC class I protein and a variant of the PSA peptide having one amino acid substitution (such as a conservative amino acid substitution). In some embodiments, the anti-PMC antibody moiety cross-reacts with at least one (such as at least any of 2, 3, 4, or 5) complex comprising the PSA peptide and a different subtype of the MHC class I protein.

In some embodiments, there is provided an anti-PMC immunoconjugate comprising a) an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions, and b) an effector molecule.

In some embodiments, there is provided an anti-PMC immunoconjugate comprising a) an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184, and b) an effector molecule.

In some embodiments, there is provided an anti-PMC immunoconjugate comprising a) an anti-PMC antibody moiety that specifically binds to a complex comprising a PSA peptide and an MHC class I protein comprising i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions, and b) an effector molecule.

In some embodiments, there is provided an anti-PMC immunoconjugate comprising a) an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences, and b) an effector molecule.

In some embodiments, there is provided an anti-PMC immunoconjugate comprising a) an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174, and b) an effector molecule.

In some embodiments, there is provided an anti-PMC immunoconjugate comprising a) an anti-PMC antibody moiety that specifically binds to a complex comprising a PSA peptide and an MHC class I protein comprising a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% (for example at least about any of 96%, 97%, 98%, or 99%) sequence identity, and b) an effector molecule.

In some embodiments, there is provided an anti-PMC immunoconjugate comprising a) an anti-PMC antibody moiety that specifically binds to a complex comprising a PSA peptide and an MHC class I protein comprising a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154, b) an effector molecule.

### Nucleic Acids

Nucleic acid molecules encoding the anti-PMC constructs or anti-PMC antibody moieties are also contemplated. In some embodiments, there is provided a nucleic acid (or a set of nucleic acids) encoding a full-length anti-PMC antibody. In some embodiments, there is provided a nucleic acid (or a set of nucleic acids) encoding a multi-specific anti-PMC molecule (e.g., a multi-specific anti-PMC antibody, a bispecific anti-PMC antibody, or a bispecific T-cell engager anti-PMC antibody), or polypeptide portion thereof. In some embodiments, there is provided a nucleic acid (or a set of nucleic acids) encoding an anti-PMC CAR. In some embodiments, there is provided a nucleic acid (or a set of nucleic acids) encoding an anti-PMC immunoconjugate, or polypeptide portion thereof.

For example, in some embodiments, there is provided a nucleic acid encoding an anti-PMC construct comprising an anti-PMC antibody moiety, wherein the anti-PMC antibody moiety comprises a heavy chain variable region and a light chain variable region, and wherein the nucleic acid comprises the nucleotide sequence of SEQ ID NO: 179 encoding the heavy chain variable region and the nucleotide sequence of SEQ ID NO: 180 encoding the light chain variable region.

The present application also includes variants to these nucleic acid sequences. For example, the variants include nucleotide sequences that hybridize to the nucleic acid sequences encoding the anti-PMC constructs or anti-PMC antibody moieties of the present application under at least moderately stringent hybridization conditions.

The present invention also provides vectors in which a nucleic acid of the present invention is inserted.

In brief summary, the expression of an anti-PMC construct (e.g., anti-PMC CAR) or polypeptide portion thereof by a natural or synthetic nucleic acid encoding the anti-PMC construct or polypeptide portion thereof can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory. New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements. e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell. e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual. Cold Spring Harbor Laboratory. New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian. e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting. RT-PCR and PCR; “biochemical” assays, such as detecting the presence or absence of a particular peptide, e.g., by immunological means (ELISAs and Western blots) or by assays described herein to identify agents falling within the scope of the invention.

### MHC Class I Proteins

MHC class I proteins are one of two primary classes of major histocompatibility complex (MHC) molecules (the other being MHC class II) and are found on nearly every nucleated cell of the body. Their function is to display fragments of proteins from within the cell to T cells; healthy cells will be ignored, while cells containing foreign proteins will be attacked by the immune system. Because MHC class I proteins present peptides derived from cytosolic proteins, the pathway of MHC class I presentation is often called the cytosolic or endogenous pathway. Class I MHC molecules bind peptides generated mainly from degradation of cytosolic proteins by the proteasome. The MHC I:peptide complex is then inserted into the plasma membrane of the cell. The peptide is bound to the extracellular part of the class I MHC molecule. Thus, the function of the class I MHC is to display intracellular proteins to cytotoxic T cells (CTLs). However, class I MHC can also present peptides generated from exogenous proteins, in a process known as cross-presentation.

MHC class I proteins consist of two polypeptide chains, α and β2-microglobulin (β2M). The two chains are linked noncovalently via interaction of b2m and the α3 domain. Only the α chain is polymorphic and encoded by a HLA gene, while the b2m subunit is not polymorphic and encoded by the β-2 microglobulin gene. The α3 domain is plasma membrane-spanning and interacts with the CD8 co-receptor of T-cells. The α3-CD8 interaction holds the MHC I molecule in place while the T cell receptor (TCR) on the surface of the cytotoxic T cell binds its α1-α2 heterodimer ligand, and checks the coupled peptide for antigenicity. The α1 and α2 domains fold to make up a groove for peptides to bind. MHC class I proteins bind peptides that are 8-10 amino acid in length.

The human leukocyte antigen (HLA) genes are the human versions of the MHC genes. The three major MHC class I proteins in humans are HLA-A, HLA-B, and HLA-C, while the 3 minor ones are HLA-E, HLA-F, and HLA-G, HLA-A is ranked among the genes in humans with the fastest-evolving coding sequence. As of December 2013, there were 2432 known HLA-A alleles coding for 1740 active proteins and 117 null proteins. The HLA-A gene is located on the short arm of chromosome 6 and encodes the larger, α-chain, constituent of HLA-A. Variation of HLA-A α-chain is key to HLA function. This variation promotes genetic diversity in the population. Since each HLA has a different affinity for peptides of certain structures, greater variety of HLAs means greater variety of antigens to be ‘presented’ on the cell surface, enhancing the likelihood that a subset of the population will be resistant to any given foreign invader. This decreases the likelihood that a single pathogen has the capability to wipe out the entire human population. Each individual can express up to two types of HLA-A, one from each of their parents. Some individuals will inherit the same HLA-A from both parents, decreasing their individual HLA diversity; however, the majority of individuals will receive two different copies of HLA-A. This same pattern follows for all HLA groups. In other words, a person can only express either one or two of the 2432 known HLA-A alleles.

All alleles receive at least a four digit classification, e.g., HLA-A*02:12. The A signifies which HLA gene the allele belongs to. There are many HLA-A alleles, so that classification by serotype simplifies categorization. The next pair of digits indicates this assignment. For example, HLA-A*02:02, HLA-A*02:04, and HLA-A*02:324 are all members of the A2 serotype (designated by the *02 prefix). This group is the primary factor responsible for HLA compatibility. All numbers after this cannot be determined by serotyping and are designated through gene sequencing. The second set of digits indicates what HLA protein is produced. These are assigned in order of discovery and as of December 2013 there are 456 different HLA-A02 proteins known (assigned names HLA-A*02:01 to HLA-A*02:456). The shortest possible HLA name includes both of these details. Each extension beyond that signifies a nucleotide change that may or may not change the protein.

In some embodiments, the anti-PMC antibody moiety specifically binds to a complex comprising a PSA peptide and an MHC class I protein, wherein the MHC class I protein is HLA-A, HLA-B, HLA-C, HLA-E, HLA-F, or HLA-G. In some embodiments, the MHC class I protein is HLA-A, HLA-B. or HLA-C. In some embodiments, the MHC class I protein is HLA-A. In some embodiments, the MHC class I protein is HLA-B. In some embodiments, the MHC class I protein is HLA-C. In some embodiments, the MHC class I protein is HLA-A01, HLA-A02, HLA-A03, HLA-A09, HLA-A10, HLA-A11, HLA-A19, HLA-A23, HLA-A24, HLA-A25, HLA-A26, HLA-A28, HLA-A29, HLA-A30, HLA-A31, HLA-A32, HLA-A33, HLA-A34, HLA-A36, HLA-A43, HLA-A66, HLA-A68, HLA-A69, HLA-A74, or HLA-A80. In some embodiments, the MHC class I protein is HLA-A02. In some embodiments, the MHC class I protein is any one of HLA-A*02:01-555, such as HLA-A*02:01, HLA-A*02:02, HLA-A*02:03, HLA-A*02:04, HLA-A*02:05, HLA-A*02:06, HLA-A*02:07, HLA-A*02:08, HLA-A*02:09, HLA-A*02:10, HLA-A*02:11, HLA-A*02:12, HLA-A*02:13, HLA-A*02:14, HLA-A*02:15, HLA-A*02:16, HLA-A*02:17, HLA-A*02:18, HLA-A*02:19, HLA-A*02:20, HLA-A*02:21, HLA-A*02:22, or HLA-A*02:24. In some embodiments, the MHC class I protein is HLA-A*02:01, HLA-A*02:01 is expressed in 39-46% of all Caucasians. and therefore represents a suitable choice of MHC class I protein for use in the present invention.

PSA peptides suitable for use in generating anti-PMC antibody moieties can be determined, for example, based on the presence of HLA-A*02:01-binding motifs and cleavage sites for proteasomes and immune-proteasomes using computer prediction models known to those of skill in the art. For predicting MHC class I binding sites, such models include, but are not limited to, IEDB (Vita et al., The immune epitope database (IEDB) 3.0. Nucleic Acids Res. 2014 Oct. 9, pii: gku938). ProPred1 (described in more detail in Singh and Raghava, ProPred: prediction of HLA-DR binding sites. BIOINFORMATICS 17(12):1236-1237, 2001), and SYFPEITHI (see Schuler et al. SYFPEITHI, Database for Searching and T-Cell Epitope Prediction, in Immunoinformatics Methods in Molecular Biology, vol 409(1): 75-93, 2007).

Once appropriate peptides have been identified, peptide synthesis may be done in accordance with protocols well known to those of skill in the art. Because of their relatively small size, the peptides of the invention may be directly synthesized in solution or on a solid support in accordance with conventional peptide synthesis techniques. Various automatic synthesizers are commercially available and can be used in accordance with known protocols. The synthesis of peptides in solution phase has become a well-established procedure for large-scale production of synthetic peptides and as such is a suitable alternative method of preparing the peptides of the invention (See for example, Solid Phase Peptide Synthesis by John Morrow Stewart and Martin et al. Application of Almez-mediated Amidation Reactions to Solution Phase Peptide Synthesis, Tetrahedron Letters Vol. 39, pages 1517-1520, 1998).

The binding activity of candidate PSA peptides can be tested using the antigen-processing-deficient T2 cell line, which increases expression of HLA-A when stabilized by a peptide in the antigen-presenting groove. T2 cells are pulsed with the candidate peptide for a time sufficient to stabilize HLA-A expression on the cell surface, which can be measured using any methods known in the art, such as by immunostaining with a fluorescently labeled monoclonal antibody specific for HLA-A (for example, BB7.2) followed by fluorescence-activated cell-sorting (FACS) analysis.

### Preparation of Anti-PMC Antibodies and Anti-PMC Antibody Moieties

In some embodiments, the anti-PMC antibody or anti-PMC antibody moiety is a monoclonal antibody. Monoclonal antibodies can be prepared, e.g., using hybridoma methods, such as those described by Kohler and Milstein, Nature. 256:495 (1975) and Sergeeva et al. Blood, 117(16):4262-4272, using the phage display methods described herein and in the Examples below, or using recombinant DNA methods (see, e.g., U.S. Pat. No. 4,816,567).

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest, or a complex comprising at least two molecules, such as a complex comprising a PSA peptide and an MHC class I protein. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. See, e.g., Goding, Monoclonal Antibodies: Principles and Practice (New York: Academic Press, 1986). pp. 59-103. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center. San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al. Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The anti-PMC antibodies or antibody moieties may also be identified by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury. Methods in Molecular Biology 248:161-175 (Lo, ed., Human Press. Totowa, N.J., 2003); Sidhu et al., J. Mol. Biol. 338(2): 299-310 (2004); Lee et al., J. Mol. Biol. 340(5): 1073-1093 (2004); Fellouse. Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The antibodies or antigen-binding fragments thereof can be prepared using phage display to screen libraries for antibodies specific to a complex comprising a PSA peptide and an MHC class I protein. The library can be a human scFv phage display library having a diversity of at least one×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a non-human phage display library.

Phage clones that bind to the PMC with high affinity can be selected by iterative binding of phage to the PMC, which is bound to a solid support (such as, for example, beads for solution panning or mammalian cells for cell panning), followed by removal of non-bound phage and by elution of specifically bound phage. In an example of solution panning, the PMC can be biotinylated for immobilization to a solid support. The biotinylated PMC is mixed with the phage library and a solid support, such as streptavidin-conjugated Dynabeads M-280, and then PMC-phage-bead complexes are isolated. The bound phage clones are then eluted and used to infect an appropriate host cell, such as E. coli XL1-Blue, for expression and purification. In an example of cell panning, T2 cells (a TAP-deficient, HLA-A*02:01⁺ lymphoblast cell line) loaded with the PSA peptide of the PMC are mixed with the phage library, after which the cells are collected and the bound clones are eluted and used to infect an appropriate host cell for expression and purification. The panning can be performed for multiple (such as about any of 2, 3, 4, 5, 6 or more) rounds with either solution panning, cell panning, or a combination of both, to enrich for phage clones binding specifically to the PMC. Enriched phage clones can be tested for specific binding to the PMC by any methods known in the art, including for example ELISA and FACS.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). Hybridoma cells as described above or PMC-specific phage clones of the invention can serve as a source of such DNA. Once isolated, the DNA can be placed into expression vectors, which are then transfected into host cells such as simian COS cells. Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a nonimmunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fe region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge. CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology, 121: 210 (1986).

### Human and Humanized Antibodies

The anti-PMC antibodies or antibody moieties can be humanized antibodies or human antibodies. Humanized forms of non-human (e.g., murine) antibodies are chimeric immunoglobulins, immunoglobulin chains, or fragments thereof (such as Fv, Fab. Fab′, F(ab′)₂, scFv, or other antigen-binding subsequences of antibodies) that typically contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin, and all or substantially all of the FR regions are those of a human immunoglobulin consensus sequence. In some embodiments, the humanized antibody will comprise at least a portion of an immunoglobulin constant region (Fc), typically that of a human immunoglobulin. See. e.g., Jones et al., Nature. 321: 522-525 (1986); Riechmann et al., Nature, 332: 323-329 (1988); Presta. Curr. Op. Struct. Biol., 2:593-596 (1992).

Generally, a humanized antibody has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature. 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibodies are antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661016, and Marks et al., Bio/Technology, 10: 779-783 (1992); Lonberg et al., Nature 368: 856-859 (1994); Morrison, Nature, 368: 812-813 (1994); Fishwild et al., Nature Biotechnology. 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al, and Boerner et al, are also available for the preparation of human monoclonal antibodies. Cole et al., Monoclonal Antibodies and Cancer Therapy. Alan R. Liss, p. 77 (1985) and Boerner et al., J. Immunol., 147(1): 86-95 (1991).

### Multi-Specific Antibodies

In some embodiments, the anti-PMC construct is a multi-specific antibody. Suitable methods for making multi-specific (e.g., bispecific) antibodies are well known in the art. For example, the production of bispecific antibodies can based on the co-expression of two immunoglobulin heavy-chain/light-chain pairs, where the two pairs each have different specificities, and upon association result in a heterodimeric antibody (see, e.g., Milstein and Cuello. Nature, 305: 537-539 (1983); WO 93/08829, and Traunecker et al., EMBO J. 10: 3655 (1991)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829 and in Traunecker et al., EMBO. 10: 3655-3659 (1991). Alternatively, the combining of heavy and light chains can be directed by taking advantage of species-restricted pairing (see, e.g., Lindhofer et al., J. Immunol., 155:219-225 (1995)) and the pairing of heavy chains can be directed by use of “knob-into hole” engineering of CH3 domains (see, e.g., U.S. Pat. No. 5,731,168; Ridgway et al., Protein Eng., 9(7):617-621 (1996)). Multi-specific antibodies may also be made by engineering electrostatic steering effects for making antibody Fc-heterodimeric molecules (see, e.g., WO 2009/089004A1). In yet another method, stable bispecific antibodies can be generated by controlled Fab-arm exchange, where two parental antibodies having distinct antigen specificity and matched point mutations in the CH3 domains are mixed in reducing condition to allow for separation, reassembly, and reoxidation to form highly pure bispecific antibodies. Labrigin et al., Proc. Natl. Acad Sci., 110(13):5145-5150 (2013). Such antibodies, comprising a mixture of heavy-chain/light-chain pairs, are also referred to herein as “heteromultimeric antibodies”.

Antibodies or antigen-binding fragments thereof having different specificities can also be chemically cross-linked to generate multi-specific heteroconjugate antibodies. For example, two F(ab′)2 molecules, each having specificity for a different antigen, can be chemically linked. Pullarkat et al., Trends Biotechnol., 48:9-21 (1999). Such antibodies have, for example, been proposed to target immune-system cells to unwanted cells (U.S. Pat. No. 4,676,980), and for treatment of HIV infection. WO 91/00360; WO 92/200373; EP 03089. It is contemplated that the antibodies can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

In some embodiments, multi-specific antibodies can be prepared using recombinant DNA techniques. For example, a bispecific antibody can be engineered by fusing two scFvs, such as by fusing them through a peptide linker, resulting in a tandem scFv. One example of a tandem scFv is a bispecific T cell engager. Bispecific T cell engagers are made by linking an anti-CD3 scFv to an scFv specific for a surface antigen of a target cell, such as a tumor-associated antigen (TAA), resulting in the redirection of T cells to the target cells. Mack et al., Proc. Natl. Acad. Sci., 92:7021-7025 (1995); Brischwein et al., Mol. Immunol., 43(8):1129-1143 (2006). By shortening the length of a peptide linker between two variable domains, they can be prevented from self-assembling and forced to pair with domains on a second polypeptide, resulting in a compact bispecific antibody called a diabody (Db). Holliger et al., Proc. Natl. Acad. Sci., 90:6444-6448 (1993). The two polypeptides of a Db each comprise a VH connected to a VL by a linker which is too short to allow pairing between the two domains on the same chain. Accordingly, the VH and VL domains of one polypeptide are forced to pair with the complementary VL and VH domains of another polypeptide, thereby forming two antigen-binding sites. In a modification of this format, the two polypeptides are linked by another peptide linker, resulting in a single chain diabody (scDb). In yet another modification of the Db format, dual-affinity retargeting (DART) bispecific antibodies can be generated by introducing a disulfide linkage between cysteine residues at the C-terminus of each polypeptide, optionally including domains prior to the C-terminal cysteine residues that drive assembly of the desired heterodimeric structure. Veri et al., Arthritis Rheum., 62(7):1933-1943 (2010). Dual-variable-domain immunoglobulins (DVD-Ig™), in which the target-binding variable domains of two monoclonal antibodies are combined via naturally occurring linkers to yield a tetravalent, bispecific antibody, are also known in the art. Gu and Ghayur, Methods Enzymol., 502:25-41 (2012). In yet another format. Dock and Lock (DNL), bispecific antibodies are prepared by taking advantage of the dimerization of a peptide (DDD2) derived from the regulatory subunit of human cAMP-dependent protein kinase (PKA) with a peptide (AD2) derived from the anchoring domains of human A kinase anchor proteins (AKAPs). Rossi et al., Proc. Natl. Acad. Sci., 103:6841-6846 (2006).

Various techniques for making and isolating bispecific antibody fragments directly from recombinant cell culture have also been described. For example, bispecific antibodies have been produced using leucine zippers. Kostelny et al., J. Immunol., 148(5): 1547-1553 (1992). This method can also be utilized for the production of antibody homodimers.

### Anti-PMC Variants

In some embodiments, amino acid sequence variants of the antibody moieties provided herein are contemplated. For example, it may be desirable to improve the binding affinity and/or other biological properties of the antibody moiety. Amino acid sequence variants of an antibody moiety may be prepared by introducing appropriate modifications into the nucleotide sequence encoding the antibody moiety, or by peptide synthesis. Such modifications include, for example, deletions from, and/or insertions into and/or substitutions of residues within the amino acid sequences of the antibody moiety. Any combination of deletion, insertion, and substitution can be made to arrive at the final construct, provided that the final construct possesses the desired characteristics, e.g., antigen-binding.

In some embodiments, antibody moiety variants having one or more amino acid substitutions are provided. Sites of interest for substitutional mutagenesis include the HVRs and FRs. Amino acid substitutions may be introduced into an antibody moiety of interest and the products screened for a desired activity. e.g., retained/improved antigen binding, decreased immunogenicity, or improved ADCC or CDC.

Conservative substitutions are shown in Table 5 below.

TABLE 5
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gin;
c. acidic: Asp, Glu;
d. basic: His. Lys, Arg;
e. residues that influence chain orientation: Gly, Pro;
f. aromatic: Trp. Tyr, Phe.

Non-conservative substitutions will entail exchanging a member of one of these classes for another class.

An exemplary substitutional variant is an affinity matured antibody moiety, which may be conveniently generated, e.g., using phage display-based affinity maturation techniques. Briefly, one or more CDR residues are mutated and the variant antibody moieties displayed on phage and screened for a particular biological activity (e.g. binding affinity). Alterations (e.g., substitutions) may be made in HVRs. e.g., to improve antibody moiety affinity. Such alterations may be made in HVR “hotspots,” i.e., residues encoded by codons that undergo mutation at high frequency during the somatic maturation process (see. e.g., Chowdhury, Methods Mol. Biol. 207:179-196 (2008)), and/or specificity determining residues (SDRs), with the resulting variant VH or VL being tested for binding affinity. Affinity maturation by constructing and reselecting from secondary libraries has been described, e.g., in Hoogenboom et al, in Methods in Molecular Biology 178:1-37 (O'Brien et al., ed., Human Press, Totowa. N.J. (2001).)

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody moiety variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling, CDR-H3 and CDR-L3 in particular are often targeted.

In some embodiments, substitutions, insertions, or deletions may occur within one or more HVRs so long as such alterations do not substantially reduce the ability of the antibody moiety to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant VH and VL sequences provided above, each HVR either is unaltered, or contains no more than one, two or three amino acid substitutions.

A useful method of identification of residues or regions of an antibody moiety that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody moiety with antigen is affected. Further substitutions may be introduced at the amino acid locations demonstrating functional sensitivity to the initial substitutions. Alternatively. or additionally, a crystal structure of an antigen-antibody moiety complex can be determined to identify contact points between the antibody moiety and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody moiety with an N-terminal methionyl residue. Other insertional variants of the antibody moiety include the fusion to the N- or C-terminus of the antibody moiety to an enzyme (e.g. for ADEPT) or a polypeptide which increases the serum half-life of the antibody moiety.

### Fc Region Variants

In some embodiments, one or more amino acid modifications may be introduced into the Fc region of a full-length anti-PMC antibody provided herein, thereby generating an Fc region variant. In some embodiments, the Fc region variant has enhanced antibody dependent cellular cytotoxicity (ADCC) effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al. J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-PMC antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is called CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the invention contemplates an anti-PMC construct variant comprising an FC region that possesses some but not all effector functions, which makes it a desirable candidate for applications in which the half-life of the anti-PMC construct in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example. Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see. e.g. Hellstrom, I. et al. Proc. Nat'l Acad Sci. USA 83:7059-7063 (1986)) and Hellstrom, I et al., Proc. Nat'l Acad Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively. or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example. Gazzano-Santoro et al., J. Immunol. Methods 202:163 (1996); Cragg, M. S. et al., Blood 101:1045-1052 (2003); and Cragg, M. S. and M. J. Glennie. Blood 103:2738-2743 (2004)). FcRn binding and in vivo clearance/half life determinations can also be performed using methods known in the art (see. e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, there is provided an anti-PMC construct (e.g., a full-length anti-PMC antibody) variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-PMC construct (e.g., full-length anti-PMC antibody) variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided an anti-PMC construct (e.g., a full-length anti-PMC antibody) variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-PMC constructs (such as full-length anti-PMC antibodies) comprising any of the Fc variants described herein, or combinations thereof, are contemplated.

### Glycosylation Variants

In some embodiments, an anti-PMC construct provided herein is altered to increase or decrease the extent to which the anti-PMC construct is glycosylated. Addition or deletion of glycosylation sites to an anti-PMC construct may be conveniently accomplished by altering the amino acid sequence of the anti-PMC construct or polypeptide portion thereof such that one or more glycosylation sites is created or removed.

Where the anti-PMC construct comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates. e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-PMC construct of the invention may be made in order to create anti-PMC construct variants with certain improved properties.

In some embodiments, anti-PMC construct (such as full-length anti-PMC antibody) variants are provided comprising an Fc region wherein a carbohydrate structure attached to the Fc region has reduced fucose or lacks fucose, which may improve ADCC function. Specifically, anti-PMC constructs are contemplated herein that have reduced fusose relative to the amount of fucose on the same anti-PMC construct produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-PMC construct is one wherein less than about 50%, 40%, 30%, 20%, 10%, or 5% of the N-linked glycans thereon comprise fucose. For example, the amount of fucose in such an anti-PMC construct may be from 1% to 80%, from 1% to 65%, from 5% to 65% or from 20% to 40%. In some embodiments, the anti-PMC construct is one wherein none of the N-linked glycans thereon comprise fucose. i.e., wherein the anti-PMC construct is completely without fucose, or has no fucose or is afucosylated. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297. i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example S11), and knockout cell lines, such as α-1,6-fucosyltransferase gene. FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-PMC construct (such as full-length anti-PMC antibody) variants are further provided with bisected oligosaccharides, e.g., in which a biantennary oligosaccharide attached to the Fc region of the anti-PMC construct is bisected by GlcNAc. Such anti-PMC construct (such as full-length anti-PMC antibody) variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-PMC construct (such as full-length anti-PMC antibody) variants with at least one galactose residue in the oligosaccharide attached to the Fc region are also provided. Such anti-PMC construct variants may have improved CDC function. Such antibody variants are described, e.g., in WO 1997/30087 (Patel et al.); WO 1998/58964 (Raju, S.); and WO 1999/22764 (Raju, S.).

In some embodiments, the anti-PMC construct (such as full-length anti-PMC antibody) variants comprising an Fc region are capable of binding to an FcγRIII. In some embodiments, the anti-PMC construct (such as full-length anti-PMC antibody) variants comprising an Fc region have ADCC activity in the presence of human effector cells or have increased ADCC activity in the presence of human effector cells compared to the otherwise same anti-PMC construct (such as full-length anti-PMC antibody) comprising a human wild-type IgG1Fc region.

### Cysteine Engineered Variants

In some embodiments, it may be desirable to create cysteine engineered anti-PMC constructs (such as full-length anti-PMC antibodies) in which one or more amino acid residues are substituted with cysteine residues. In some embodiments, the substituted residues occur at accessible sites of the anti-PMC construct. By substituting those residues with cysteine, reactive thiol groups are thereby positioned at accessible sites of the anti-PMC construct and may be used to conjugate the anti-PMC construct to other moieties, such as drug moieties or linker-drug moieties, to create an anti-PMC immunoconjugate, as described further herein. Cysteine engineered anti-PMC constructs (such as full-length anti-PMC antibodies) may be generated as described. e.g., in U.S. Pat. No. 7,521,541.

### Derivatives

In some embodiments, an anti-PMC construct provided herein may be further modified to contain additional nonproteinaceous moieties that are known in the art and readily available. The moieties suitable for derivatization of the anti-PMC construct include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-PMC construct may vary, and if more than one polymer are attached, they can be the same or different molecules. In general, the number and/or type of polymers used for derivatization can be determined based on considerations including, but not limited to, the particular properties or functions of the anti-PMC construct to be improved, whether the anti-PMC construct derivative will be used in a therapy under defined conditions, etc.

In some embodiments, conjugates of an anti-PMC construct and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the anti-PMC construct-nonproteinaceous moiety are killed.

### CAR Effector Cell Preparation

The present invention in one aspect provides effector cells (such as lymphocytes, for example T cells) expressing an anti-PMC CAR. Exemplary methods of preparing effector cells (such as T cells) expressing the anti-PMC CARs (anti-PMC CAR effector cells, such as anti-PMC CAR T cells) are provided herein.

In some embodiments, an anti-PMC CAR effector cell (such as T cell) can be generated by introducing a vector (including for example a lentiviral vector) comprising an anti-PMC CAR (for example a CAR comprising an anti-PMC antibody moiety and CD28 and CD3ζ intracellular signaling sequences) into the effector cell (such as T cell). In some embodiments, the anti-PMC CAR effector cells (such as T cells) of the invention are able to replicate in vivo, resulting in long-term persistence that can lead to sustained control of a PSA-positive disease (such as cancer. e.g., prostate cancer, breast cancer, ovarian cancer, or lung cancer).

In some embodiments, the invention relates to administering a genetically modified T cell expressing an anti-PMC CAR for the treatment of a patient having a PSA-positive disease or at risk of having a PSA-positive disease using lymphocyte infusion. In some embodiments, autologous lymphocyte infusion is used in the treatment. Autologous PBMCs are collected from a patient in need of treatment and T cells are activated and expanded using the methods described herein and known in the art and then infused back into the patient.

In some embodiments, the anti-PMC CAR T cell expresses an anti-PMC CAR comprising an anti-PMC antibody moiety (also referred to herein as an “anti-PMC CAR T cell”). In some embodiments, the anti-PMC CAR T cell expresses an anti-PMC CAR comprising an extracellular domain comprising an anti-PMC antibody moiety and an intracellular domain comprising intracellular signaling sequences of CD3ζ and CD28 or 4-1BB. The anti-PMC CAR T cells of the invention can undergo robust in vivo T cell expansion and can establish PMC-specific memory cells that persist at high levels for an extended amount of time in blood and bone marrow. In some embodiments, the anti-PMC CAR T cells of the invention infused into a patient can eliminate PMC-presenting cells, such as PMC-presenting cancer cells, in vivo in patients having a PSA-positive disease. In some embodiments, the anti-PMC CAR T cells of the invention infused into a patient can eliminate PMC-presenting cells, such as PMC-presenting cancer cells, in vivo in patients having a PSA-positive disease that is refractory to at least one conventional treatment.

Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of T cell lines available in the art may be used. In some embodiments of the present invention, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least one, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD11b, CD16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells. B cells, dendritic cells, and other cells of the immune system.

Whether prior to or after genetic modification of the T cells to express a desirable anti-PMC CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular. T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

### Immunoconjugate Preparation

The anti-PMC immunoconjugates may be prepared using any methods known in the art. See, e.g., WO 2009/067800, WO 2011/133886, and U.S. Patent Application Publication No. 2014322129, incorporated by reference herein in their entirety.

The anti-PMC antibody moiety of an anti-PMC immunoconjugate may be “attached to” the effector molecule by any means by which the anti-PMC antibody moiety can be associated with, or linked to, the effector molecule. For example, the anti-PMC antibody moiety of an anti-PMC immunoconjugate may be attached to the effector molecule by chemical or recombinant means. Chemical means for preparing fusions or conjugates are known in the art and can be used to prepare the anti-PMC immunoconjugate. The method used to conjugate the anti-PMC antibody moiety and effector molecule must be capable of joining the binding protein with the effector molecule without interfering with the ability of the binding protein to bind to the antigen on the target cell.

The anti-PMC antibody moiety of an anti-PMC immunoconjugate may be linked indirectly to the effector molecule. For example, the anti-PMC antibody moiety of an anti-PMC immunoconjugate may be directly linked to a liposome containing the effector molecule of one of several types. The effector molecule(s) and/or the anti-PMC antibody moiety may also be bound to a solid surface.

In some embodiments, the anti-PMC antibody moiety of an anti-PMC immunoconjugate and the effector molecule are both proteins and can be conjugated using techniques well known in the art. There are several hundred crosslinkers available that can conjugate two proteins. (See for example “Chemistry of Protein Conjugation and Crosslinking”. 1991, Shans Wong, CRC Press. Ann Arbor). The crosslinker is generally chosen based on the reactive functional groups available or inserted on the anti-PMC antibody moiety and/or effector molecule. In addition, if there are no reactive groups, a photoactivatible crosslinker can be used. In certain instances, it may be desirable to include a spacer between the anti-PMC antibody moiety and the effector molecule. Crosslinking agents known to the art include the homobifunctional agents: glutaraldehyde, dimethyladipimidate and Bis(diazobenzidine) and the heterobifunctional agents: m Maleimidobenzoyl-N-Hydroxysuccinimide and Sulfo-m Maleimidobenzoyl-N-Hydroxysuccinimide.

In some embodiments, the anti-PMC antibody moiety of an anti-PMC immunoconjugate may be engineered with specific residues for chemical attachment of the effector molecule. Specific residues used for chemical attachment of molecule known to the art include lysine and cysteine. The crosslinker is chosen based on the reactive functional groups inserted on the anti-PMC antibody moiety, and available on the effector molecule.

An anti-PMC immunoconjugate may also be prepared using recombinant DNA techniques. In such a case a DNA sequence encoding the anti-PMC antibody moiety is fused to a DNA sequence encoding the effector molecule, resulting in a chimeric DNA molecule. The chimeric DNA sequence is transfected into a host cell that expresses the fusion protein. The fusion protein can be recovered from the cell culture and purified using techniques known in the art.

Examples of attaching an effector molecule, which is a label, to the binding protein include the methods described in Hunter, et al., Nature 144:945 (1962); David, et al., Biochemistry 13:1014 (1974); Pain, et al., J. Immunol. Meth. 40:219 (1981); Nygren, J. Histochem. and Cytochem. 30:407 (1982): Wensel and Meares. Radioimmunoimaging And Radioimmunotherapy, Elsevier, N.Y. (1983); and Colcher et al., “Use Of Monoclonal Antibodies As Radiopharmaceuticals For The Localization Of Human Carcinoma Xenografts In Athymic Mice”, Meth. Enzymol., 121:802-16 (1986).

The radio- or other labels may be incorporated in the immunoconjugate in known ways. For example, the peptide may be biosynthesized or may be synthesized by chemical amino acid synthesis using suitable amino acid precursors involving, for example, fluorine-19 in place of hydrogen. Labels such as ⁹⁹Tc or ¹²³I, ¹⁸⁶Re, ¹⁸⁸Re and ¹¹¹In can be attached via a cysteine residue in the peptide. Yttrium-90 can be attached via a lysine residue. The IODOGEN method (Fraker et al., Biochem. Biophys. Res. Commun. 80:49-57 (1978)) can be used to incorporate iodine-123. “Monoclonal Antibodies in Immunoscintigraphy” (Chatal. CRC Press 1989) describes other methods in detail.

Immunoconjugates of the antibody moiety and a cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene tnaminepentaacetic acid (MX-DTPA) is an exemplary chelating agent for conjugation of radionucleotide to the antibody. See, e.g., WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The anti-PMC immunoconjugates of the invention expressly contemplate, but are not limited to, ADC prepared with cross-linker reagents: BMPS, EMCS, GMBS, HBVS, LC-SMCC, MBS, MPBH, SBAP. SIA. SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology. Inc., Rockford. Ill., U.S.A). See pages 467-498, 2003-2004 Applications Handbook and Catalog.

### Pharmaceutical Compositions

Also provided herein are compositions (such as pharmaceutical compositions, also referred to herein as formulations) comprising an anti-PMC construct. In some embodiments, the composition further comprises a cell (such as an effector cell. e.g., a T cell) associated with the anti-PMC construct. In some embodiments, there is provided a pharmaceutical composition comprising an anti-PMC construct and a pharmaceutically acceptable carrier. In some embodiments, the pharmaceutical composition further comprises a cell (such as an effector cell, e.g., a T cell) associated with the anti-PMC construct. In yet other embodiments, there is provided a composition (such as a pharmaceutical composition) comprising a nucleic acid encoding an anti-PMC construct.

Suitable formulations of the anti-PMC constructs are obtained by mixing an anti-PMC construct having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-PMC constructs of this invention into cells.

The formulation herein may also contain one or more active compounds in addition to the anti-PMC construct as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent in addition to the anti-PMC construct. Such molecules are suitably present in combination in amounts that are effective for the purpose intended. The effective amount of such other agents depends on the amount of anti-PMC construct present in the formulation, the type of disease or disorder or treatment, and other factors discussed above. These are generally used in the same dosages and with administration routes as described herein or about from 1 to 99% of the heretofore employed dosages.

The anti-PMC constructs may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Sustained-release preparations may be prepared.

Sustained-release preparations of the anti-PMC constructs can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydro gels release proteins for shorter time periods. When encapsulated antibodies remain in the body for a long time, they can denature or aggregate as a result of exposure to moisture at 37° C. resulting in a loss of biological activity and possible changes in immunogenicity. Rational strategies can be devised for stabilization of anti-PMC constructs depending on the mechanism involved. For example, if the aggregation mechanism is discovered to be intermolecular S-S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-PMC construct is formulated in a buffer comprising a citrate, NaCl, acetate, succinate, glycine, polysorbate 80 (Tween 80), or any combination of the foregoing. In some embodiments, the anti-PMC construct is formulated in a buffer comprising about 100 mM to about 150 mM glycine. In some embodiments, the anti-PMC construct is formulated in a buffer comprising about 50 mM to about 100 mM NaCl. In some embodiments, the anti-PMC construct is formulated in a buffer comprising about 10 mM to about 50 mM acetate. In some embodiments, the anti-PMC construct is formulated in a buffer comprising about 10 mM to about 50 mM succinate. In some embodiments, the anti-PMC construct is formulated in a buffer comprising about 0.005% to about 0.02% polysorbate 80. In some embodiments, the anti-PMC construct is formulated in a buffer having a pH between about 5.1 and 5.6. In some embodiments, the anti-PMC construct is formulated in a buffer comprising 10 mM citrate. 100 mM NaCl, 100 mM glycine, and 0.01% polysorbate 80, wherein the formulation is at pH 5.5.

The formulations to be used for in vivo administration must be sterile. This is readily accomplished by, e.g., filtration through sterile filtration membranes.

### Methods for Treatment Using Anti-PMC Constructs

The anti-PMC constructs and/or compositions of the invention can be administered to individuals (e.g., mammals such as humans) to treat a disease and/or disorder associated with PSA overexpression (also referred to herein as a “PSA-positive” disease or disorder), including, for example, PSA-positive cancer (such as prostate cancer, breast cancer, ovarian cancer, or lung cancer). The present application thus in some embodiments provides a method of treating a PSA-positive disease (such as cancer) in an individual comprising administering to the individual an effective amount of a composition (such as a pharmaceutical composition) comprising an anti-PMC construct comprising an anti-PMC antibody moiety, such as any one of the anti-PMC constructs described herein. In some embodiments, the composition further comprises a cell (such as an effector cell) associated with the anti-PMC construct. In some embodiments, the cancer is selected, for example, from the group consisting of prostate cancer, breast cancer, ovarian cancer, and lung cancer.

For example, in some embodiments, there is provided a method of treating a PSA-positive disease in an individual comprising administering to the individual an effective amount of a composition comprising an anti-PMC construct comprising an anti-PMC antibody moiety that specifically binds to a complex comprising a PSA peptide and an MHC class I protein. In some embodiments, the PSA peptide is PSA 146-154 (SEQ ID NO: 4). In some embodiments, the MHC class I protein is HLA-A02. In some embodiments, the MHC class I protein is HLA-A*02:01. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the composition further comprises a cell (such as an effector cell) associated with the anti-PMC construct. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an anti-PMC construct comprising an anti-PMC antibody moiety that specifically binds to a complex comprising a PSA 146-154 peptide (SEQ ID NO: 4) and HLA-A*02:01. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the composition further comprises a cell (such as an effector cell) associated with the anti-PMC construct. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an anti-PMC construct comprising an anti-PMC antibody moiety that specifically binds to a complex comprising a PSA peptide and an MHC class I protein, wherein the anti-PMC antibody moiety comprises: i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the composition further comprises a cell (such as an effector cell) associated with the anti-PMC construct. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an anti-PMC construct comprising an anti-PMC antibody moiety that specifically binds to a complex comprising a PSA peptide and an MHC class I protein, wherein the anti-PMC antibody moiety comprises: i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the composition further comprises a cell (such as an effector cell) associated with the anti-PMC construct. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an anti-PMC construct comprising an anti-PMC antibody moiety that specifically binds to a complex comprising a PSA peptide and an MHC class I protein, wherein the anti-PMC antibody moiety comprises: i) a heavy chain variable domain sequence comprising an HC-CDR1 comprising the amino acid sequence of any one of SEQ ID NOs: 40-52, 155, and 156, or a variant thereof comprising up to about 5 (for example about any of 1, 2, 3, 4, or 5) amino acid substitutions; an HC-CDR2 comprising the amino acid sequence of any one of SEQ ID NOs: 53-65 and 157, or a variant thereof comprising up to about 5 (for example about any of 1, 2, 3, 4, or 5) amino acid substitutions; and an HC-CDR3 comprising the amino acid sequence of any one of SEQ ID NOs: 66-78 and 158-162; or a variant thereof comprising up to about 5 (for example about any of 1, 2, 3, 4, or 5) amino acid substitutions; and ii) a light chain variable domain sequence comprising an LC-CDR1 comprising the amino acid sequence of any one of SEQ ID NOs: 79-91 and 163-166, or a variant thereof comprising up to about 5 (for example about any of 1, 2, 3, 4, or 5) amino acid substitutions; an LC-CDR2 comprising the amino acid sequence of any one of SEQ ID NOs: 92-104 and 167-169, or a variant thereof comprising up to about 3 (for example about any of 1, 2, or 3) amino acid substitutions; and an LC-CDR3 comprising the amino acid sequence of any one of SEQ ID NOs: 105-117 and 170-174; or a variant thereof comprising up to about 5 (for example about any of 1, 2, 3, 4, or 5) amino acid substitutions. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the composition further comprises a cell (such as an effector cell) associated with the anti-PMC construct. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an anti-PMC construct comprising an anti-PMC antibody moiety that specifically binds to a complex comprising a PSA peptide and an MHC class I protein, wherein the anti-PMC antibody moiety comprises: i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (for example about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (for example about any of 1, 2, 3, 4, or 5) amino acid substitutions in the LC-CDR sequences. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the composition further comprises a cell (such as an effector cell) associated with the anti-PMC construct. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an anti-PMC construct comprising an anti-PMC antibody moiety that specifically binds to a complex comprising a PSA peptide and an MHC class I protein, wherein the anti-PMC antibody moiety comprises: i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the composition further comprises a cell (such as an effector cell) associated with the anti-PMC construct. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an anti-PMC construct comprising an anti-PMC antibody moiety that specifically binds to a complex comprising a PSA peptide and an MHC class I protein, wherein the anti-PMC antibody moiety comprises a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% (for example at least about any of 96%, 97%, 98%, or 99%) sequence identity. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the composition further comprises a cell (such as an effector cell) associated with the anti-PMC construct. In some embodiments, the PSA-opposite disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an anti-PMC construct comprising an anti-PMC antibody moiety that specifically binds to a complex comprising a PSA peptide and an MHC class I protein, wherein the anti-PMC antibody moiety comprises a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the composition further comprises a cell (such as an effector cell) associated with the anti-PMC construct. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments of any of the methods for treating a PSA-positive disease described herein, the anti-PMC construct is conjugated to a cell (such as an immune cell. e.g., a T cell) prior to being administered to the individual. Thus, for example, there is provided a method of treating a PSA-positive disease in an individual comprising a) conjugating any one of the anti-PMC constructs described herein to a cell (such as an immune cell, e.g., a T cell) to form an anti-PMC construct/cell conjugate, and b) administering to the individual an effective amount of a composition comprising the anti-PMC construct/cell conjugate. In some embodiments, the cell is derived from the individual. In some embodiments, the cell is not derived from the individual. In some embodiments, the anti-PMC construct is conjugated to the cell by covalent linkage to a molecule on the surface of the cell. In some embodiments, the anti-PMC construct is conjugated to the cell by non-covalent linkage to a molecule on the surface of the cell. In some embodiments, the anti-PMC construct is conjugated to the cell by insertion of a portion of the anti-PMC construct into the outer membrane of the cell. In some embodiments, the anti-PMC construct is non-naturally occurring. In some embodiments, the anti-PMC construct is a full-length antibody. In some embodiments, the anti-PMC construct is a multi-specific (such as bispecific) molecule. In some embodiments, the anti-PMC construct is a chimeric antigen receptor. In some embodiments, the anti-PMC construct is an immunoconjugate. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or genetically prone to one or more of the diseases or disorders described herein (such as prostate cancer, breast cancer, ovarian cancer, or lung cancer). In some embodiments, the individual has one or more risk factors associated with one or more diseases or disorders described herein.

The present application in some embodiments provides a method of delivering an anti-PMC construct (such as any one of the anti-PMC constructs described herein) to a cell presenting on its surface a complex comprising a PSA peptide and an MHC class I protein in an individual, the method comprising administering to the individual a composition comprising the anti-PMC construct. In some embodiments, the anti-PMC construct to be delivered is associated with a cell (such as an effector cell, e.g., a T cell).

Many diagnostic methods for PSA-positive cancer (such as prostate cancer, breast cancer, ovarian cancer, or lung cancer) or any other disease exhibiting PSA expression and the clinical delineation of those diseases are known in the art. Such methods include, but are not limited to, e.g., immunohistochemistry, PCR, and fluorescent in situ hybridization (FISH).

In some embodiments, the anti-PMC constructs and/or compositions of the invention are administered in combination with a second, third, or fourth agent (including. e.g., an antineoplastic agent, a growth inhibitory agent, a cytotoxic agent, or a chemotherapeutic agent) to treat diseases or disorders involving PSA expression. In some embodiments, the anti-PMC construct is administered in combination with an agent that increases the expression of MHC class I proteins and/or enhances the surface presentation of PSA peptides by MHC class I proteins. In some embodiments, the agent includes, for example, IFN receptor agonists. Hsp90 inhibitors, enhancers of p53 expression, and chemotherapeutic agents. In some embodiments, the agent is an IFN receptor agonist including, for example, IFNγ, IFNβ, and IFNα. In some embodiments, the agent is an Hsp90 inhibitor including, for example, tanespimycin (17-AAG), alvespimycin (17-DMAG), retaspimycin (IPI-504), IPI-493. CNF2024/BIIB021. MPC-3100, Debio 0932 (CUDC-305), PU-H71, Ganetespib (STA-9090), NVP-AUY922 (VER-52269), HSP990, KW-2478, AT13387, SNX-5422, DS-2248, and XL888. In some embodiments, the agent is an enhancer of p53 expression including, for example, 5-fluorouracil and nutlin-3. In some embodiments, the agent is a chemotherapeutic agent including, for example, topotecan, etoposide, cisplatin, paclitaxel, and vinblastine.

In some embodiments, there is provided a method of treating a PSA-positive disease in an individual, wherein the cells expressing PSA do not normally present, or present at relatively low levels, a complex comprising a PSA protein and an MHC class I protein on their surface, the method comprising administering to the individual a composition comprising an anti-PMC construct in combination with an agent that increases the expression of MHC class I proteins and/or enhances the surface presentation of PSA peptides by MHC class I proteins. In some embodiments, the agent includes, for example, IFN receptor agonists, Hsp90 inhibitors, enhancers of p53 expression, and chemotherapeutic agents. In some embodiments, the agent is an IFN receptor agonist including, for example, IFNγ. IFNβ, and IFNα. In some embodiments, the agent is an Hsp90 inhibitor including, for example, tanespimycin (17-AAG), alvespimycin (17-DMAG), retaspimycin (IPI-504), IPI-493, CNF2024/BIIB021. MPC-3100, Debio 0932 (CUDC-305), PU-H71, Ganetespib (STA-9090), NVP-AUY922 (VER-52269). HSP990, KW-2478, AT13387, SNX-5422. DS-2248, and XL888. In some embodiments, the agent is an enhancer of p53 expression including, for example, 5-fluorouracil and nutlin-3. In some embodiments, the agent is a chemotherapeutic agent including, for example, topotecan, etoposide, cisplatin, paclitaxel, and vinblastine.

Cancer treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

In some embodiments, the efficacy of treatment is measured as the percentage tumor growth inhibition (% TGI), calculated using the equation 100−(T/C×100), where T is the mean relative tumor volume of the treated tumor, and C is the mean relative tumor volume of a non-treated tumor. In some embodiments, the % TGI is about 10%, about 20%, about 30%, about 40%, about 50%, about 60%, about 70%, about 80%, about 90%, about 91%, about 92%, about 93%, about 94%, about 95%, or more than 95%.

### Dosing and Method of Administering the Anti-PMC Construct Compositions

The dose of the anti-PMC construct compositions administered to an individual (such as a human) may vary with the particular composition, the mode of administration, and the type of disease being treated. In some embodiments, the amount of the composition is effective to result in an objective response (such as a partial response or a complete response). In some embodiments, the amount of the anti-PMC construct composition is sufficient to result in a complete response in the individual. In some embodiments, the amount of the anti-PMC construct composition is sufficient to result in a partial response in the individual. In some embodiments, the amount of the anti-PMC construct composition administered (for example when administered alone) is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the anti-PMC construct composition. Responses of an individual to the treatment of the methods described herein can be determined, for example, based on RECIST levels.

In some embodiments, the amount of the composition is sufficient to prolong progress-free survival of the individual. In some embodiments, the amount of the composition is sufficient to prolong overall survival of the individual. In some embodiments, the amount of the composition (for example when administered along) is sufficient to produce clinical benefit of more than about any of 50%, 60%, 70%, or 77% among a population of individuals treated with the anti-PMC construct composition.

In some embodiments, the amount of the composition, alone or in combination with a second, third, and/or fourth agent, is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the anti-PMC construct (e.g., full-length anti-PMC antibody, multi-specific anti-PMC molecule, anti-PMC CAR, or anti-PMC immunoconjugate) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the composition is close to a maximum tolerated dose (MTD) of the composition following the same dosing regimen. In some embodiments, the amount of the composition is more than about any of 80%, 90%, 95%, or 98% of the MTD.

In some embodiments, the amount of an anti-PMC construct (e.g., full-length anti-PMC antibody, multi-specific anti-PMC molecule, anti-PMC CAR, or anti-PMC immunoconjugate) in the composition is included in a range of about 0.001 μg to about 1000 μg.

In some embodiments of any of the above aspects, the effective amount of an anti-PMC construct (e.g., full-length anti-PMC antibody, multi-specific anti-PMC molecule, anti-PMC CAR, or anti-PMC immunoconjugate) in the composition is in the range of about 0.1 μg/kg to about 100 mg/kg of total body weight.

The anti-PMC construct compositions can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In some embodiments, the composition is administered intravenously. In some embodiments, the composition is administered intraportally. In some embodiments, the composition is administered intraarterially. In some embodiments, the composition is administered intraperitoneally. In some embodiments, the composition is administered intrahepatically. In some embodiments, the composition is administered by hepatic arterial infusion.

### Anti-PMC CAR Effector Cell Therapy

The present application also provides methods of using an anti-PMC CAR to redirect the specificity of an effector cell (such as a primary T cell) to a complex comprising a PSA peptide and an MHC class I protein. Thus, the present invention also provides a method of stimulating an effector cell-mediated response (such as a T cell-mediated immune response) to a target cell population or tissue comprising PMC-presenting cells in a mammal, comprising the step of administering to the mammal an effector cell (such as a T cell) that expresses an anti-PMC CAR.

Anti-PMC CAR effector cells (such as T cells) expressing the anti-PMC CAR can be infused to a recipient in need thereof. The infused cell is able to kill PMC-presenting cells in the recipient. In some embodiments, unlike antibody therapies, anti-PMC CAR effector cells (such as T cells) are able to replicate in vivo resulting in long-term persistence that can lead to sustained tumor control.

In some embodiments, the anti-PMC CAR effector cells are anti-PMC CAR T cells that can undergo robust in vivo T cell expansion and can persist for an extended amount of time. In some embodiments, the anti-PMC CAR T cells of the invention develop into specific memory T cells that can be reactivated to inhibit any additional tumor formation or growth.

The anti-PMC CAR T cells of the invention may also serve as a type of vaccine for ex vivo immunization and/or in vivo therapy in a mammal. In some embodiments, the mammal is a human.

With respect to ex vivo immunization, of least one of the following occurs in vitro prior to administering the cell into a mammal: i) expansion of the cells, ii) introducing a nucleic acid encoding an anti-PMC CAR to the cells, and/or iii) cryopreservation of the cells.

Ex vivo procedures are well known in the art and are discussed more fully below. Briefly, cells are isolated from a mammal (preferably a human) and genetically modified (i.e., transduced or transfected in vitro) with a vector expressing an anti-PMC CAR disclosed herein. The anti-PMC CAR cell can be administered to a mammalian recipient to provide a therapeutic benefit. The mammalian recipient may be a human and the anti-PMC CAR cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34⁺ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

The anti-PMC CAR effector cells (such as T cells) of the present invention may be administered either alone, or as a pharmaceutical composition in combination with diluents and/or with other components such as IL-2 or other cytokines or cell populations. Briefly, pharmaceutical compositions of the present invention may comprise anti-PMC CAR effector cells (such as T cells), in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, anti-PMC CAR effector cell (such as T cell) compositions are formulated for intravenous administration.

The precise amount of the anti-PMC CAR effector cell (such as T cell) compositions of the present invention to be administered can be determined by a physician with consideration of individual differences in age, weight, tumor size, extent of infection or metastasis, and condition of the patient (subject). In some embodiments, a pharmaceutical composition comprising the anti-PMC CAR effector cells (such as T cells) is administered at a dosage of about 10⁴ to about 10⁹ cells/kg body weight, such any of about 10⁴ to about 10, about 10⁵ to about 10⁶, about 10⁶ to about 10⁷, about 10⁸ to about 10⁸, or about 10⁸ to about 10⁹ cells/kg body weight, including all integer values within those ranges. Anti-PMC CAR effect cell (such as T cell) compositions may also be administered multiple times at these dosages. The cells can be administered by using infusion techniques that are commonly known in immunotherapy (see. e.g., Rosenberg et al., New Eng. J. of Med. 319:1676, 1988). The optimal dosage and treatment regimen for a particular patient can readily be determined by one skilled in the art of medicine by monitoring the patient for signs of disease and adjusting the treatment accordingly.

In some embodiments, it may be desired to administer activated anti-PMC CAR T cells to a subject and then subsequently redraw blood (or have an apheresis performed), activate T cells therefrom according to the present invention, and reinfuse the patient with these activated and expanded T cells. This process can be carried out multiple times every few weeks. In some embodiments. T cells can be activated from blood draws of from 10 cc to 400 cc. In some embodiments, T cells are activated from blood draws of 20 cc, 30 cc, 40 cc, 50 cc. 60 cc. 70 cc, 80 cc, 90 cc, or 100 cc.

The administration of the anti-PMC CAR effector cells (such as T cells) may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In some embodiments, the anti-PMC CAR effector cell (such as T cell) compositions of the present invention are administered to a patient by intradermal or subcutaneous injection. In some embodiments, the anti-PMC CAR effector cell (such as T cell) compositions of the present invention are administered by i.v. injection. The compositions of anti-PMC CAR effector cells (such as T cells) may be injected directly into a tumor, lymph node, or site of infection.

Thus, for example, in some embodiments, there is provided a method of treating a PSA-positive disease in an individual comprising administering to the individual an effective amount of a composition comprising an effector cell (such as a T cell) expressing an anti-PMC CAR comprising a) an extracellular domain comprising an anti-PMC antibody moiety that specifically binds to a complex comprising a PSA peptide and an MHC class I protein, b) a transmembrane domain, and c) an intracellular signaling domain. In some embodiments, the intracellular signaling domain comprises a CD3ζ intracellular signaling sequence. In some embodiments, the intracellular signaling domain further comprises a co-stimulatory sequence, including, without limitation, a CD28 or 4-1BB intracellular signaling sequence. In some embodiments, the PSA peptide is PSA 146-154 (SEQ ID NO: 4). In some embodiments, the MHC class I protein is HLA-A02. In some embodiments, the MHC class I protein is HLA-A*02:01. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an effector cell (such as a T cell) expressing an anti-PMC CAR comprising a) an extracellular domain comprising an anti-PMC antibody moiety that specifically binds to a complex comprising a PSA 146-154 peptide (SEQ ID NO: 4) and HLA-A*02:01, b) a transmembrane domain, and c) an intracellular signaling domain comprising a CD3ζ intracellular signaling sequence and a CD28 or 4-1BB intracellular signaling sequence. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an effector cell (such as a T cell) expressing an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, or a variant thereof comprising up to about 3 (for example about any of 1, 2, or 3) amino acid substitutions, an HC-CDR2 comprising the amino acid sequence of SEQ ID NO: 119, or a variant thereof comprising up to about 3 (for example about any of 1, 2, or 3) amino acid substitutions, and an HC-CDR3 comprising the amino acid sequence of SEQ ID NO: 120 or 181, or a variant thereof comprising up to about 3 (for example about any of 1, 2, or 3) amino acid substitutions; and ii) a light chain variable domain comprising an LC-CDR1 comprising the amino acid sequence of SEQ ID NO: 121, or a variant thereof comprising up to about 3 (for example about any of 1, 2, or 3) amino acid substitutions, and an LC-CDR3 comprising the amino acid sequence of SEQ ID NO: 122, 123, 124, 182, 183, or 184, or a variant thereof comprising up to about 3 (for example about any of 1, 2, or 3) amino acid substitutions, b) a transmembrane domain, and c) an intracellular signaling domain comprising a CD3ζ intracellular signaling sequence and a CD28 or 4-1BB intracellular signaling sequence. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an effector cell (such as a T cell) expressing an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of SEQ ID NO: 118, an HC-CDR2 comprising the amino acid sequence of SEQ ID NO: 119, and an HC-CDR3 comprising the amino acid sequence of SEQ ID NO: 120 or 181; and ii) a light chain variable domain comprising an LC-CDR1 comprising the amino acid sequence of SEQ ID NO: 121, and an LC-CDR3 comprising the amino acid sequence of SEQ ID NO: 122, 123, 124, 182, 183, or 184, b) a transmembrane domain, and c) an intracellular signaling domain comprising a CD3ζ intracellular signaling sequence and a CD28 or 4-1BB intracellular signaling sequence. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an effector cell (such as a T cell) expressing an anti-PMC CAR comprising a) an extracellular domain comprising an anti-PMC antibody moiety that specifically binds to a complex comprising a PSA peptide and an MHC class I protein comprising i) a heavy chain variable domain comprising an HC-CDR1 comprising the amino acid sequence of any one of SEQ ID NOs: 40-52, 155, and 156, or a variant thereof comprising up to about 5 (such as about any of 1, 2, 3, 4, or 5) amino acid substitutions, an HC-CDR2 comprising the amino acid sequence of any one of SEQ ID NOs: 53-65 and 157, or a variant thereof comprising up to about 5 (such as about any of 1, 2, 3, 4, or 5) amino acid substitutions, and an HC-CDR3 comprising the amino acid sequence of any one of SEQ ID NOs: 66-78 and 158-162, or a variant thereof comprising up to about 5 (such as about any of 1, 2, 3, 4, or 5) amino acid substitutions; and ii) a light chain variable domain comprising an LC-CDR1 comprising the amino acid sequence of any one of SEQ ID NOs: 79-91 and 163-166, or a variant thereof comprising up to about 5 (such as about any of 1, 2, 3, 4, or 5) amino acid substitutions, an LC-CDR2 comprising the amino acid sequence of any one of SEQ ID NOs: 92-104 and 167-169, or a variant thereof comprising up to about 3 (such as about any of 1, 2, or 3) amino acid substitutions, and an LC-CDR3 comprising the amino acid sequence of any one of SEQ ID NOs: 105-117 and 170-174, or a variant thereof comprising up to about 5 (such as about any of 1, 2, 3, 4, or 5) amino acid substitutions; b) a transmembrane domain, and c) an intracellular signaling domain comprising a CD3ζ intracellular signaling sequence and a CD28 or 4-1BB intracellular signaling sequence. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an effector cell (such as a T cell) expressing an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; or a variant thereof comprising up to about 5 (such as about any of 1, 2, 3, 4, or 5) amino acid substitutions in the HC-CDR sequences; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; or a variant thereof comprising up to about 5 (such as about any of 1, 2, 3, 4, or 5) amino acid substitutions in the LC-CDR sequences; b) a transmembrane domain, and c) an intracellular signaling domain comprising a CD3ζ intracellular signaling sequence and a CD28 or 4-1BB intracellular signaling sequence. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an effector cell (such as a T cell) expressing an anti-PMC CAR comprising a) an extracellular domain comprising an anti-PMC antibody moiety that specifically binds to a complex comprising a PSA peptide and an MHC class I protein comprising i) a heavy chain variable domain sequence comprising an HC-CDR1 comprising the amino acid sequence of any one of SEQ ID NOs: 40-52, 155, and 156; an HC-CDR2 comprising the amino acid sequence of any one of SEQ ID NOs: 53-65 and 157; and an HC-CDR3 comprising the amino acid sequence of any one of SEQ ID NOs: 66-78 and 158-162; and ii) a light chain variable domain sequence comprising an LC-CDR1 comprising the amino acid sequence of any one of SEQ ID NOs: 79-91 and 163-166; an LC-CDR2 comprising the amino acid sequence of any one of SEQ ID NOs: 92-104 and 167-169; and an LC-CDR3 comprising the amino acid sequence of any one of SEQ ID NOs: 105-117 and 170-174; b) a transmembrane domain, and c) an intracellular signaling domain comprising a CD3ζ intracellular signaling sequence and a CD28 or 4-1BB intracellular signaling sequence. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an effector cell (such as a T cell) expressing an anti-PMC CAR comprising a) an extracellular domain comprising an anti-PMC antibody moiety that specifically binds to a complex comprising a PSA peptide and an MHC class I protein comprising i) a heavy chain variable domain comprising the amino acid sequence of any one of SEQ ID NOs: 14-26 and 145-149, or a variant thereof having at least about 95% (for example at least about any of 96%, 97%, 98%, or 99%) sequence identity, and a light chain variable domain comprising the amino acid sequence of any one of SEQ ID NOs: 27-39 and 150-154, or a variant thereof having at least about 95% sequence identity; b) a transmembrane domain, and c) an intracellular signaling domain comprising a CD3ζ intracellular signaling sequence and a CD28 or 4-1BB intracellular signaling sequence. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

In some embodiments, there is provided a method of treating a PSA-positive disease in an individual comprising administering to the individual an effective amount of a composition comprising an effector cell (such as a T cell) expressing an anti-PMC CAR comprising a) an extracellular domain comprising an anti-PMC antibody moiety that specifically binds to a complex comprising a PSA peptide and an MHC class I protein comprising a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154; b) a transmembrane domain, and c) an intracellular signaling domain comprising a CD3ζ intracellular signaling sequence and a CD28 or 4-1BB intracellular signaling sequence. In some embodiments, the PSA-positive disease is cancer. In some embodiments, the cancer is, for example, prostate cancer, breast cancer, ovarian cancer, or lung cancer. In some embodiments, the cancer is prostate cancer, such as hormone-resistant prostate cancer. In some embodiments, the individual is human.

### Cancers

The anti-PMC constructs and anti-PMC CAR cells in some embodiments can be useful for treating PSA-positive cancer. Cancers that may be treated using any of the methods described herein include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the anti-PMC constructs and anti-PMC CAR cells of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), bladder carcinoma, melanoma, cancer of the uterus (e.g., endometrial carcinoma), urothelial cancers (e.g., squamous cell carcinoma, transitional cell carcinoma, adenocarcinoma, ureter cancer, and urinary bladder cancer), and CNS tumors (such as a glioma (such as brainstem glioma and mixed gliomas), glioblastoma (also known as glioblastoma multiforme) astrocytoma. CNS lymphoma, germinoma, medulloblastoma. Schwannoma craniopharyogioma, ependymoma, pinealoma, hemangioblastoma, acoustic neuroma, oligodendroglioma, menangioma, neuroblastoma, retinoblastoma and brain metastases).

In some embodiments, the PSA-overexpression cancer is prostate cancer. In some embodiments, the prostate cancer is an adenocarcinoma. In some embodiments, the prostate cancer is a sarcoma, neuroendocrine tumor, small cell cancer, ductal cancer, or a lymphoma. In some embodiments, the prostate cancer is at any of the four stages. A, B, C. or D, according to the Jewett staging system. In some embodiments, the prostate cancer is stage A prostate cancer (e.g., the cancer cannot be felt during a rectal exam). In some embodiments, the prostate cancer is stage B prostate cancer (e.g., the tumor involves more tissue within the prostate, and can be felt during a rectal exam, or is found with a biopsy that is done because of a high PSA level). In some embodiments, the prostate cancer is stage C prostate cancer (e.g., the cancer has spread outside the prostate to nearby tissues). In some embodiments, the prostate cancer is stage D prostate cancer. In some embodiments, the prostate cancer is androgen independent prostate cancer (AIPC). In some embodiments, the prostate cancer is androgen dependent prostate cancer. In some embodiments, the prostate cancer is refractory to hormone therapy.

Cancer treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

### Methods for Diagnosis and Imaging Using Anti-PMC Constructs

Labeled anti-PMC antibody moieties and derivatives and analogs thereof, which specifically bind to a PMC on the surface of a cell, can be used for diagnostic purposes to detect, diagnose, or monitor diseases and/or disorders associated with the expression, aberrant expression and/or activity of PSA, including any of the diseases and disorders described herein, such as cancer (e.g., prostate cancer, breast cancer, ovarian cancer, or lung cancer). For example, the anti-PMC antibody moieties of the invention can be used in in situ, in vivo, ex vivo, and in vitro diagnostic assays or imaging assays.

Additional embodiments of the invention include methods of diagnosing a disease or disorder associated with expression or aberrant expression of PSA in an individual (e.g., a mammal, such as a human). The methods comprise detecting PMC-presenting cells in the individual. In some embodiments, there is provided a method of diagnosing a disease or disorder associated with expression or aberrant expression of PSA in an individual (e.g., a mammal, such as a human) comprising (a) administering an effective amount of a labeled anti-PMC antibody moiety according to any of the embodiments described herein to the individual; and (b) determining the level of the label in the individual, such that a level of the label above a threshold level indicates that the individual has the disease or disorder. The threshold level can be determined by various methods, including, for example, by detecting the label according to the method of diagnosing described herein in a first set of individuals that have the disease or disorder and a second set of individuals that do not have the disease or disorder, and setting the threshold to a level that allows for discrimination between the first and second sets. In some embodiments, the threshold level is zero, and the method comprises determining the presence or absence of the label in the individual. In some embodiments, the method further comprises waiting for a time interval following the administering of step (a) to permit the labeled anti-PMC antibody moiety to preferentially concentrate at sites in the individual where the PMC is expressed (and for unbound labeled anti-PMC antibody moiety to be cleared). In some embodiments, the method further comprises subtracting a background level of the label. Background level can be determined by various methods, including, for example, by detecting the label in the individual prior to administration of the labeled anti-PMC antibody moiety, or by detecting the label according to the method of diagnosing described herein in an individual that does not have the disease or disorder. In some embodiments, the disease or disorder is cancer. In some embodiments, the cancer is selected, for example, from the group consisting of hepatocellular carcinoma, germ cell tumor, and breast cancer. In some embodiments, the cancer is hepatocellular carcinoma. In some embodiments, the cancer is metastatic hepatocellular carcinoma. In some embodiments, the cancer is metastatic hepatocellular carcinoma, and the method further comprises determining the level of the label in the individual's blood. In some embodiments, the individual is human.

In some embodiments, there is provided a method of diagnosing a metastatic PSA-positive cancer (such as metastatic prostate cancer) in an individual (e.g., a mammal, such as a human), comprising (a) administering an effective amount of a labeled anti-PMC antibody moiety according to any of the embodiments described herein to the individual; and (b) determining the level of the label in the individual's blood, such that a level of the label above a threshold level indicates that the individual has a metastatic PSA-positive cancer. The threshold level can be determined by various methods, including, for example, by detecting the label according to the method of diagnosing described herein in a first set of individuals that have a metastatic PSA-positive cancer and a second set of individuals that do not have a metastatic PSA-positive cancer, and setting the threshold to a level that allows for discrimination between the first and second sets. In some embodiments, the threshold level is zero, and the method comprises determining the presence or absence of the label in the individual's blood. In some embodiments, the method further comprises waiting for a time interval following the administering of step (a) to permit the labeled anti-PMC antibody moiety to preferentially concentrate at sites in the individual where the PMC is expressed (and for unbound labeled anti-PMC antibody moiety to be cleared). In some embodiments, the method further comprises subtracting a background level of the label. Background level can be determined by various methods, including, for example, by detecting the label in the individual prior to administration of the labeled anti-PMC antibody moiety, or by detecting the label according to the method of diagnosing described herein in an individual that does not have a metastatic PSA-positive cancer. In some embodiments, the individual is human.

In some embodiments, there is provided a method of diagnosing a disease or disorder associated with expression or aberrant expression of PSA in an individual (e.g., a mammal, such as a human), comprising (a) contacting a labeled anti-PMC antibody moiety according to any of the embodiments described herein with a sample (such as whole blood or homogenized tissue) derived from the individual; and (b) determining the number of cells bound with the labeled anti-PMC antibody moiety in the sample, such that a value for the number of cells bound with the labeled anti-PMC antibody moiety above a threshold level indicates that the individual has the disease or disorder. The threshold level can be determined by various methods, including, for example, by determining the number of cells bound with the labeled anti-PMC antibody moiety according to the method of diagnosing described herein in a first set of individuals that have the disease or disorder and a second set of individuals that do not have the disease or disorder, and setting the threshold to a level that allows for discrimination between the first and second sets. In some embodiments, the threshold level is zero, and the method comprises determining the presence or absence of cells bound with the labeled anti-PMC antibody moiety in the sample. In some embodiments, the method further comprises subtracting a background level of the number of cells bound with the labeled anti-PMC antibody moiety. Background level can be determined by various methods, including, for example, by determining the number of cells bound with the labeled anti-PMC antibody moiety in the individual prior to administration of the labeled anti-PMC antibody moiety, or by determining the number of cells bound with the labeled anti-PMC antibody moiety according to the method of diagnosing described herein in an individual that does not have the disease or disorder. In some embodiments, the disease or disorder is cancer. In some embodiments, the cancer is selected, for example, from the group consisting of prostate cancer, breast cancer, ovarian cancer, and lung cancer. In some embodiments, the cancer is metastatic. In some embodiments, the cancer is metastatic prostate cancer. In some embodiments, the cancer is metastatic and the sample is a blood sample (such as whole blood). In some embodiments, the individual is human.

In some embodiments, there is provided a method of diagnosing a metastatic PSA-positive cancer in an individual (e.g., a mammal, such as a human), comprising (a) contacting a labeled anti-PMC antibody moiety according to any of the embodiments described herein with a sample (such as whole blood) derived from the individual; and (b) determining the number of cells bound with the labeled anti-PMC antibody moiety in the sample, such that a value for the number of cells bound with the labeled anti-PMC antibody moiety herein a threshold level indicates that the individual has a metastatic PSA-positive cancer. The threshold level can be determined by various methods, including, for example, by determining the number of cells bound with the labeled anti-PMC antibody moiety according to the method of diagnosing described herein in a first set of individuals that have a metastatic PSA-positive cancer and a second set of individuals that do not have a metastatic PSA-positive cancer, and setting the threshold to a level that allows for discrimination between the first and second sets. In some embodiments, the threshold level is zero, and the method comprises determining the presence or absence of cells bound with the labeled anti-PMC antibody moiety in the sample. In some embodiments, the method further comprises subtracting a background level of the number of cells bound with the labeled anti-PMC antibody moiety. Background level can be determined by various methods, including, for example, by determining the number of cells bound with the labeled anti-PMC antibody moiety in the individual prior to administration of the labeled anti-PMC antibody moiety, or by determining the number of cells bound with the labeled anti-PMC antibody moiety according to the method of diagnosing described herein in an individual that does not have a metastatic PSA-positive cancer. In some embodiments, the sample is blood (such as whole blood). In some embodiments, the individual is human.

Anti-PMC antibody moieties of the invention can be used to assay levels of PMC-presenting cell in a biological sample using methods known to those of skill in the art. Suitable antibody labels are known in the art and include enzyme labels, such as, glucose oxidase; radioisotopes, such as iodine (¹³¹I, ¹²⁵I, ¹²³I, ¹²¹I), carbon (¹⁴C), sulfur (³⁵S), tritium (³H), indium (^{115m}In, ^{113m}In, ¹¹²In, ¹¹¹In), technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), samarium (¹⁵³Sm), lutetium (¹⁷⁷Lu), gadolinium (¹⁵⁹Gd), promethium (¹⁴⁹Pm), lanthanum (¹⁴⁰La), ytterbium (¹⁷⁵Yb), holmium (¹⁶⁶Ho), yttrium (⁹⁰Y), scandium (⁴⁷Sc), rhenium (¹⁸⁶Re, ¹⁸⁸Re), praseodymium (¹⁴²Pr), rhodium (¹⁰⁵Rh), and ruthenium (⁹⁷Ru); luminol; fluorescent labels, such as fluorescein and rhodamine; and biotin.

Techniques known in the art may be applied to labeled anti-PMC antibody moieties of the invention. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003). Aside from the above assays, various in vivo and ex vivo assays are available to the skilled practitioner. For example, one can expose cells within the body of the subject to an anti-PMC antibody moiety which is optionally labeled with a detectable label, e.g., a radioactive isotope, and binding of the anti-PMC antibody moiety to the cells can be evaluated, e.g., by external scanning for radioactivity or by analyzing a sample (e.g., a biopsy or other biological sample) derived from a subject previously exposed to the anti-PMC antibody moiety.

### Articles of Manufacture and Kits

In some embodiments of the invention, there is provided an article of manufacture containing materials useful for the treatment of a PSA-positive disease such as cancer (for example prostate cancer, breast cancer, ovarian cancer, or lung cancer), for delivering an anti-PMC construct to a cell presenting a PMC on its surface, or for isolation or detection of PMC-presenting cells in an individual. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an anti-PMC construct of the invention. The label or package insert indicates that the composition is used for treating the particular condition. The label or package insert will further comprise instructions for administering the anti-PMC construct composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating PSA-positive cancer (such as prostate cancer, breast cancer, ovarian cancer, or lung cancer).

Additionally, the article of manufacture may further comprise a second container comprising a pharmaceutically-acceptable buffer, such as bacteriostatic water for injection (BWFI), phosphate-buffered saline. Ringer's solution and dextrose solution. It may further include other materials desirable from a commercial and user standpoint, including other buffers, diluents, filters, needles, and syringes.

Kits are also provided that are useful for various purposes, e.g., for treatment of a PSA-positive disease or disorder described herein, for delivering an anti-PMC construct to a cell presenting a PMC on its surface, or for isolation or detection of PMC-presenting cells in an individual, optionally in combination with the articles of manufacture. Kits of the invention include one or more containers comprising an anti-PMC construct composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-PMC construct (e.g., a full-length anti-PMC antibody, a multi-specific anti-PMC molecule (such as a bispecific anti-PMC antibody), or an anti-PMC immunoconjugate). In some embodiments, the kit comprises a) a composition comprising an anti-PMC construct, and b) an effective amount of at least one other agent, wherein the other agent increases the expression of MHC class I proteins and/or enhances the surface presentation of PSA peptides by MHC class I proteins (e.g., IFNγ, IFNβ, IFNα, or Hsp90 inhibitor). In some embodiments, the kit comprises a) a composition comprising an anti-PMC construct, and b) instructions for administering the anti-PMC construct composition to an individual for treatment of a PSA-positive disease, including for example prostate cancer, breast cancer, ovarian cancer, or lung cancer. In some embodiments, the kit comprises a) a composition comprising an anti-PMC construct, b) an effective amount of at least one other agent, wherein the other agent increases the expression of MHC class I proteins and/or enhances the surface presentation of PSA peptides by MHC class I proteins (e.g., IFNγ. IFNβ. IFNα, or Hsp90 inhibitor), and c) instructions for administering the anti-PMC construct composition and the other agent(s) to an individual for treatment of a PSA-positive disease, including for example prostate cancer, breast cancer, ovarian cancer, or lung cancer. The anti-PMC construct and the other agent(s) can be present in separate containers or in a single container. For example, the kit may comprise one distinct composition or two or more compositions wherein one composition comprises an anti-PMC construct and another composition comprises another agent.

In some embodiments, the kit comprises a) a composition comprising an anti-PMC construct (e.g., a full-length anti-PMC antibody, a multi-specific anti-PMC molecule (such as a bispecific anti-PMC antibody), or an anti-PMC immunoconjugate), and b) instructions for combining the anti-PMC construct with cells (such as cells, e.g., immune cells, derived from an individual) to form a composition comprising anti-PMC construct/cell conjugates and administering the anti-PMC construct/cell conjugate composition to the individual for treatment of a PSA-positive disease (including for example prostate cancer, breast cancer, ovarian cancer, or lung cancer). In some embodiments, the kit comprises a) a composition comprising an anti-PMC construct, and b) a cell (such as a cytotoxic cell). In some embodiments, the kit comprises a) a composition comprising an anti-PMC construct, b) a cell (such as a cytotoxic cell), and c) instructions for combining the anti-PMC construct with the cell to form a composition comprising anti-PMC construct/cell conjugates and administering the anti-PMC construct/cell conjugate composition to an individual for the treatment of a PSA-positive disease, including for example prostate cancer, breast cancer, ovarian cancer, or lung cancer. In some embodiments, the kit comprises a composition comprising an anti-PMC construct in association with a cell (such as a cytotoxic cell). In some embodiments, the kit comprises a) a composition comprising an anti-PMC construct in association with a cell (such as a cytotoxic cell), and b) instructions for administering the composition to an individual for the treatment of a PSA-positive disease, including for example prostate cancer, breast cancer, ovarian cancer, or lung cancer. In some embodiments, the association is by conjugation of the anti-PMC construct to a molecule on the surface of the cell. In some embodiments, the association is by insertion of a portion of the anti-PMC construct into the outer membrane of the cell.

In some embodiments, the kit comprises a nucleic acid (or set of nucleic acids) encoding an anti-PMC construct (e.g., a full-length anti-PMC antibody, a multi-specific anti-PMC molecule (such as a bispecific anti-PMC antibody), an anti-PMC CAR, or an anti-PMC immunoconjugate) or polypeptide portions thereof. In some embodiments, the kit comprises a) a nucleic acid (or set of nucleic acids) encoding an anti-PMC construct or polypeptide portions thereof, and b) a host cell (such as an effector cell) for expressing the nucleic acid (or set of nucleic acids). In some embodiments, the kit comprises a) a nucleic acid (or set of nucleic acids) encoding an anti-PMC construct or polypeptide portions thereof, and b) instructions for i) expressing the anti-PMC construct in a host cell (such as an effector cell. e.g., a T cell), ii) preparing a composition comprising the anti-PMC construct or the host cell expressing the anti-PMC construct, and iii) administering the composition comprising the anti-PMC construct or the host cell expressing the anti-PMC construct to an individual for the treatment of a PSA-positive disease, including for example prostate cancer, breast cancer, ovarian cancer, or lung cancer. In some embodiments, the host cell is derived from the individual. In some embodiments, the kit comprises a) a nucleic acid (or set of nucleic acids) encoding an anti-PMC construct or polypeptide portions thereof, b) a host cell (such as an effector cell) for expressing the nucleic acid (or set of nucleic acids), and c) instructions for i) expressing the anti-PMC construct in the host cell, ii) preparing a composition comprising the anti-PMC construct or the host cell expressing the anti-PMC construct, and iii) administering the composition comprising the anti-PMC construct or the host cell expressing the anti-PMC construct to an individual for the treatment of a PSA-positive disease, including for example prostate cancer, breast cancer, ovarian cancer, or lung cancer.

In some embodiments, the kit comprises a nucleic acid encoding an anti-PMC CAR. In some embodiments, the kit comprises a vector comprising a nucleic acid encoding an anti-PMC CAR. In some embodiments, the kit comprises a) a vector comprising a nucleic acid encoding an anti-PMC CAR, and b) instructions for i) introducing the vector into effector cells, such as T cells derived from an individual, ii) preparing a composition comprising the anti-PMC CAR effector cells, and iii) administering the anti-PMC CAR effector cell composition to the individual for treatment of a PSA-positive disease, including for example prostate cancer, breast cancer, ovarian cancer, or lung cancer.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the anti-PMC construct compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an anti-PMC construct (e.g., a full-length anti-PMC antibody, a multi-specific anti-PMC molecule (such as a bispecific anti-PMC antibody), an anti-PMC CAR, or an anti-PMC immunoconjugate) as disclosed herein to provide effective treatment of an individual for an extended period, such as any of a week, 8 days, 9 days, 10 days, 11 days. 12 days, 13 days, 2 weeks, 3 weeks, 4 weeks, 6 weeks, 8 weeks. 3 months, 4 months, 5 months, 7 months, 8 months, 9 months, or more. Kits may also include multiple unit doses of the anti-PMC construct and pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXEMPLARY EMBODIMENTS

### Embodiment 1

In some embodiments there is provided an isolated anti-PMC construct comprising an antibody moiety that specifically binds to a complex comprising a prostate-specific antigen (PSA) peptide and a major histocompatibility (MHC) class I protein (a PSA/MHC class I complex, or PMC).

### Embodiment 2

In some further embodiments of embodiment 1, the PSA/MHC class I complex is present on a cell surface.

### Embodiment 3

In some further embodiments of embodiment 1, the PSA/MHC class I complex is present on the surface of a cancer cell.

### Embodiment 4

In some further embodiments of any one of embodiments 1-3, the MHC class I protein is human leukocyte antigen (HLA)-A.

### Embodiment 5

In some further embodiments of embodiment 4, the MHC class I protein is HLA-A02.

### Embodiment 6

In some further embodiments of embodiment 5, the MHC class I protein is the HLA-A*02:01 subtype of the HLA-A02 allele.

### Embodiment 7

In some further embodiments of any one of embodiments 1-6, the antibody moiety cross-reacts with a complex comprising the PSA peptide and a second MHC class I protein having a different HLA allele than the MHC class I protein.

### Embodiment 8

In some further embodiments of any one of embodiments 1-7, the PSA peptide is 8 to 12 amino acids in length.

### Embodiment 9

In some further embodiments of any one of embodiments 1-8, the PSA peptide is derived from human PSA.

### Embodiment 10

In some further embodiments of any one of embodiments 1-9, the PSA peptide has an amino acid sequence selected from the group consisting of SEQ ID NOs: 3-13.

### Embodiment 11

In some further embodiments of embodiment 9, the PSA peptide has the amino acid sequence of KLQCVDLHV (SEQ ID NO: 4).

### Embodiment 12

In some further embodiments of any one of embodiments 1-11, the isolated anti-PMC construct cross-reacts with a complex comprising an interspecies variant of the PSA peptide and the MHC class I protein.

### Embodiment 13

In some further embodiments of any one of embodiments 1-12, the antibody moiety is human, humanized, or semi-synthetic.

### Embodiment 14

In some further embodiments of any one of embodiments 1-13, the antibody moiety is a full-length antibody, a Fab, a Fab′, a (Fab′)2, an Fv, or a single chain Fv (scFv).

### Embodiment 15

In some further embodiments of any one of embodiments 1-14, the antibody moiety binds to the PSA/MHC class I complex with an equilibrium dissociation constant (K_{d}) from about 0.1 pM to about 500 nM.

### Embodiment 16

In some further embodiments of any one of embodiments 1-15, the isolated anti-PMC construct binds to the PSA/MHC class I complex with a K_{d} from about 0.1 pM to about 500 nM.

### Embodiment 17

In some further embodiments of any one of embodiments 1-16, the antibody moiety comprises:

i) a heavy chain variable domain comprising a heavy chain complementarity determining region (HC-CDR) 1 comprising the amino acid sequence of G-G/Y-S/T-F-S/T-S-Y-A/G (SEQ ID NO: 118), or a variant thereof comprising up to about 3 amino acid substitutions, an HC-CDR2 comprising the amino acid sequence of I-X-P-X-X-G-X-T (SEQ ID NO: 119), or a variant thereof comprising up to about 3 amino acid substitutions, and an HC-CDR3 comprising the amino acid sequence of A-R-X-X-Y (SEQ ID NO: 120) or A-R-Y-X-F/W/Y-D (SEQ ID NO: 181); or a variant thereof comprising up to about 3 amino acid substitutions; and

ii) a light chain variable domain comprising a light chain complementarity determining region (LC-CDR) 1 comprising the amino acid sequence of S/T-S-N-F/I-G-A/N/S-G/N-Y (SEQ ID NO: 121), or a variant thereof comprising up to about 3 amino acid substitutions, and an LC-CDR3 comprising the amino acid sequence of A/G-A/T-W-D/S-S-L-N/S-A/G-X-V (SEQ ID NO: 122), L-L-Y-X-G-G (SEQ ID NO: 123). Q-S-Y-D-S/T-S-L-S-G-X-V (SEQ ID NO: 124), A/G-X-W-D/E-X-S-L (SEQ ID NO: 182). L-L/V-F/Y-X-G-G (SEQ ID NO: 183), or S/T-W/Y-X-S/T-S-L (SEQ ID NO: 184); or a variant thereof comprising up to 3 amino acid substitutions, wherein

X can be any amino acid.

### Embodiment 18

In some further embodiments of any one of embodiments 1-16, the antibody moiety comprises:

i) a heavy chain variable domain comprising an HC-CDR1 comprising the amino acid sequence of any one of SEQ ID NOs: 40-52, 155, and 156, or a variant thereof comprising up to about 5 amino acid substitutions, an HC-CDR2 comprising the amino acid sequence of any one of SEQ ID NOs: 53-65 and 157, or a variant thereof comprising up to about 5 amino acid substitutions, and an HC-CDR3 comprising the amino acid sequence of any one of SEQ ID NOs: 66-78 and 158-162; or a variant thereof comprising up to about 5 amino acid substitutions; and
ii) a light chain variable domain comprising an LC-CDR1 comprising the amino acid sequence of any one of SEQ ID NOs: 79-91 and 163-166, or a variant thereof comprising up to about 5 amino acid substitutions, an LC-CDR2 comprising the amino acid sequence of any one of SEQ ID NOs: 92-104 and 167-169, or a variant thereof comprising up to about 3 amino acid substitutions, and an LC-CDR3 comprising the amino acid sequence of any one of SEQ ID NOs: 105-117 and 170-174; or a variant thereof comprising up to about 5 amino acid substitutions.

### Embodiment 19

In some further embodiments of any one of embodiments 1-16, the antibody moiety comprises:

i) a heavy chain (HC) variable domain comprising an HC-CDR1 comprising the amino acid sequence of any one of SEQ ID NOs: 40-52, 155, and 156, an HC-CDR2 comprising the amino acid sequence of any one of SEQ ID NOs: 53-65 and 157, and an HC-CDR3 comprising the amino acid sequence of any one of SEQ ID NOs: 66-78 and 158-162; or a variant thereof comprising up to about 5 amino acid substitutions in the HC-CDR regions; and
ii) a light chain (LC) variable domain comprising an LC-CDR comprising the amino acid sequence of any one of SEQ ID NOs: 79-91 and 163-166, an LC-CDR2 comprising the amino acid sequence of any one of SEQ ID NOs: 92-104 and 167-169, and an LC-CDR3 comprising the amino acid sequence of any one of SEQ ID NOs: 105-117 and 170-174; or a variant thereof comprising up to about 5 amino acid substitutions in the LC-CDR regions.

### Embodiment 20

In some further embodiments of embodiment 18 or 19, the antibody moiety comprises a) a heavy chain variable domain comprising the amino acid sequence of any one of SEQ ID NOs: 14-26 and 145-149, or a variant thereof having at least about 95% sequence identify to any one of SEQ ID NOs: 14-26 and 145-149; and b) a light chain variable domain comprising the amino acid sequence of any one of SEQ ID NOs: 27-39 and 150-154, or a variant thereof having at least about 95% sequence identity to any one of SEQ ID NOs: 27-39 and 150-154.

### Embodiment 21

In some further embodiments of embodiment 20, the antibody moiety comprises a heavy chain variable domain comprising the amino acid sequence of any one of SEQ ID NOs: 14-26 and 145-149 and a light chain variable domain comprising the amino acid sequence of any one of SEQ ID NOs: 27-39 and 150-154.

### Embodiment 22

In some further embodiments of any one of embodiments 1-21, the isolated anti-PMC construct is a full-length antibody.

### Embodiment 23

In some further embodiments of any one of embodiments 1-22, the isolated anti-PMC construct is monospecific.

### Embodiment 24

In some further embodiments of any one of embodiments 1-22, the isolated anti-PMC construct is multispecific.

### Embodiment 25

In some further embodiments of embodiment 24, the isolated anti-PMC construct is bispecific.

### Embodiment 26

In some further embodiments of embodiment 24 or 25, the isolated anti-PMC construct is a tandem scFv, a diabody (Db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody.

### Embodiment 27

In some further embodiments of embodiment 26, the isolated anti-PMC construct is a tandem scFv comprising two scFvs linked by a peptide linker.

### Embodiment 28

In some further embodiments of embodiment 27, the peptide linker comprises the amino acid sequence GGGGS (SEQ ID NO: 175).

### Embodiment 29

In some further embodiments of any one of embodiments 24-28, the isolated anti-PMC construct further comprises a second antibody moiety that specifically binds to a second antigen.

### Embodiment 30

In some further embodiments of embodiment 29, the second antigen is an antigen on the surface of a T cell.

### Embodiment 31

In some further embodiments of embodiment 30, the second antigen is selected from the group consisting of CD3γ, CD3δ, CD3ε, CD3ζ, CD28, OX40. GITR, CD137, CD27, CD40L. and HVEM.

### Embodiment 32

In some further embodiments of embodiment 30, the second antigen is CD3ζ, and wherein the isolated anti-PMC construct is a tandem scFv comprising an N-terminal scFv specific for the PSA/MHC class I complex and a C-terminal scFv specific for CD3ε.

### Embodiment 33

In some further embodiments of embodiment 30, the T cell is selected from the group consisting of a cytotoxic T cell, a helper T cell, and a natural killer T cell.

### Embodiment 34

In some further embodiments of embodiment 29, the second antigen is an antigen on the surface of a natural killer cell, a neutrophil, a monocyte, a macrophage or a dendritic cell.

### Embodiment 35

In some further embodiments of any one of embodiments 1-21, the isolated anti-PMC construct is a chimeric antigen receptor.

### Embodiment 36

In some further embodiments of embodiment 35, the chimeric antigen receptor comprises an extracellular domain comprising the antibody moiety, a transmembrane domain, and an intracellular signaling domain comprising a CD3 intracellular signaling sequence and a CD28 or 4-1BB intracellular signaling sequence.

### Embodiment 37

In some further embodiments of any one of embodiments 1-21, the isolated anti-PMC construct is an immunoconjugate comprising the antibody moiety and an effector molecule.

### Embodiment 38

In some further embodiments of embodiment 36, the effector molecule is a therapeutic agent selected from the group consisting of a drug, a toxin, a radioisotope, a protein, a peptide, and a nucleic acid.

### Embodiment 39

In some further embodiments of embodiment 38, the therapeutic agent is a drug or a toxin.

### Embodiment 40

In some further embodiments of embodiment 37, the effector molecule is a label.

### Embodiment 41

In some embodiments there is provided a nucleic acid encoding the polypeptide components of the isolated anti-PMC construct of any one of embodiments 1-40.

### Embodiment 42

In some embodiments there is provided a vector comprising the nucleic acid of embodiment 41.

### Embodiment 43

In some embodiments there is provided a pharmaceutical composition comprising the isolated anti-PMC construct of any one of embodiments 1-39, the nucleic acid of embodiment 41, or the vector of embodiment 42.

### Embodiment 44

In some embodiments there is provided a host cell expressing the isolated anti-PMC construct of any one of embodiments 1-40.

### Embodiment 45

In some embodiments there is provided an effector cell expressing the isolated anti-PMC construct of embodiment 35 or 36.

### Embodiment 46

In some further embodiments of embodiment 45, the effector cell is a T cell.

### Embodiment 47

In some embodiments there is provided a method of detecting a cell presenting a complex comprising a PSA peptide and an MHC class I protein on its surface, comprising contacting the cell with the isolated anti-PMC construct of embodiment 39 and detecting the presence of the label on the cell.

### Embodiment 48

In some embodiments there is provided a method of treating an individual having a PSA-positive disease, comprising administering to the individual an effective amount of the pharmaceutical composition of embodiment 43.

### Embodiment 49

In some embodiments there is provided a method of treating an individual having a PSA-positive disease, comprising administering to the individual an effective amount of the effector cell of embodiment 45 or 46.

### Embodiment 50

In some further embodiments of embodiment 48 or 49, the administration is via intravenous route.

### Embodiment 51

In some further embodiments of embodiment 48 or 49, the administration is via intratumoral route.

### Embodiment 52

In some embodiments there is provided a method of diagnosing an individual having a PSA-positive disease, comprising:

a) administering an effective amount of the isolated anti-PMC construct of embodiment 40 to the individual; and
b) determining the level of the label in the individual, wherein a level of the label above a threshold level indicates that the individual has the PSA-positive disease.

### Embodiment 53

In some embodiments there is provided a method of diagnosing an individual having a PSA-positive disease, comprising:

a) contacting a sample derived from the individual with the isolated anti-PMC construct of embodiment 40; and

b) determining the number of cells bound with the isolated anti-PMC construct in the sample, wherein a value for the number of cells bound with the isolated anti-PMC construct above a threshold level indicates that the individual has the PSA-positive disease.

### Embodiment 54

In some further embodiments of any one of embodiments 48-53, the PSA-positive disease is cancer.

### Embodiment 55

In some further embodiments of embodiment 54, the cancer is selected from the group consisting of prostate cancer, breast cancer, ovarian cancer, and lung cancer.

### Embodiment 56

In some further embodiments of embodiment 55, where the cancer is prostate cancer.

### Embodiment 57

In some further embodiments of embodiment 56, the prostate cancer is hormone-resistant prostate cancer.

### EXAMPLES

### Materials

Cell Samples. Cell Lines, and Antibodies

The cell lines include: prostate cancer cell lines LNCaP (ATCC CRL-1740; HLA-A2⁺, PSA⁺), MDA PCa 2b (ATCC CRL-2422; HLA-A2⁻, PSA⁻), VCaP (ATCC CRL-2876; HLA-A2⁻, PSA⁻), 22Rv1 (ATCC CRL-2505; HLA-A2⁻, PSA⁻), and PC3 (ATCC CRL-1435; HLA-A2⁻, PSA⁻); breast cancer cell lines MDA-MB-231 (ATCC HTB-26; HLA-A2, PSA) and MDA-MB-231+PSA minigene (HLA-A2⁺. PSA⁺); liver cancer cell lines SK-Hep1 (ATCC HTB-52; HLA-A2⁺, PSA) and SK-Hep1+PSA minigene (HLA-A2⁺, PSA⁺); and lymphoblast cell line T2 (ATCC CRL-1992; HLA-A2⁺, PSA⁻). T2 is a TAP-deficient cell line. The cell lines were cultured in RPMI 1640 supplemented with 5% FCS, penicillin, streptomycin, 2 mmol/L glutamine, and 2-mercaptoethanol at 37° C./5% CO₂.

All peptides were purchased and synthesized by Genemed Synthesis, Inc. (San Antonio, Tex.). Peptides were >90% pure. The peptides were dissolved in DMSO and diluted in saline at 5 mg/mL and frozen at −180° C. Biotinylated single chain PSA peptide/HLA-A*02:01 and control peptides/HLA-A*02:01 complexes were synthesized by refolding the peptides with recombinant HLA-A02 and beta-2 microglobulin (β2M) (˜2M). 19 control peptides (p19, SEQ ID NOs: 125-143) that bind HLA-A*02:01 were derived from the following 15 genes: BCR, BTG2, CALR, CD247, CSF2RA. CTSG, DDX5, DMTN, HLA-E, IFI30, IL7, PIMI, PPP2RIB, RPS6KB1, SSR1.

### Example 1. Production of Biotinylated PSA146/HLA-A*02:01 Complex Monomer

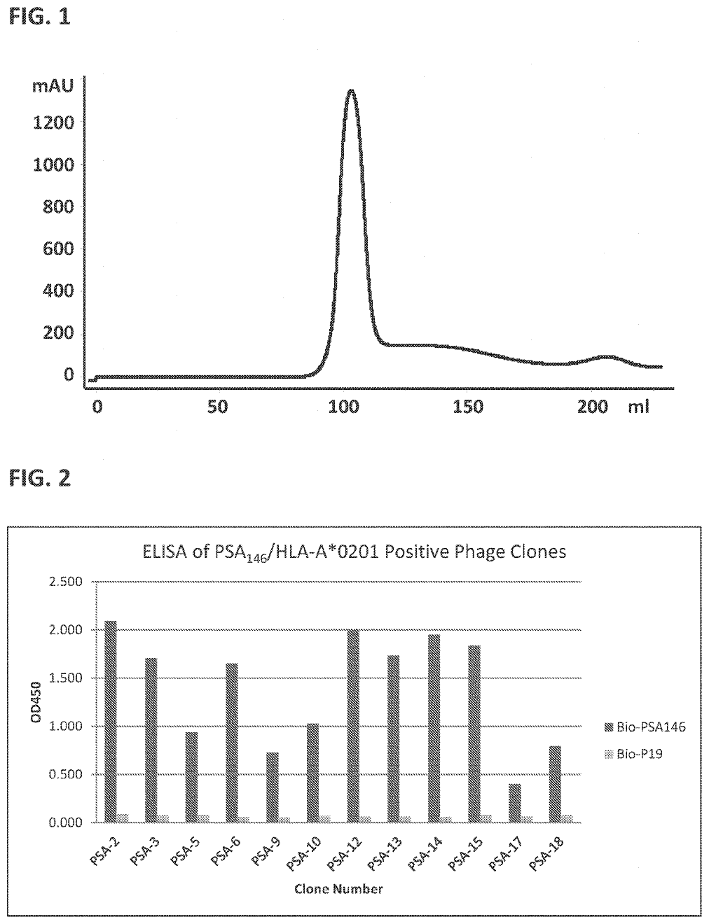

Biotinylated PSA 146-154 wild type and C9V mutant peptide/HLA-A*02:01 complex monomers were prepared according to standard protocols (John D. Altman and Mark M. Davis, Current Protocols in Immunology 17.3.1-17.3.33, 2003). In brief, DNA encoding full-length human beta-2 microglobulin (β2m) was synthesized by Genewiz and cloned into vector pET-27b. The BirA substrate peptide (BSP) was added to the C-terminus of HLA-A*02:01 extracellular domain (ECD). DNA encoding HLA-A*02:01 ECD-BSP was also synthesized by Genewiz and cloned into vector pET-27b. The vectors expressing human β2m and HLA-A*02:01 ECD-BSP were transformed into E. coli BL21 cells separately, and expressed proteins were isolated as inclusion bodies from bacterial culture. Peptide ligand PSA 146-154 was refolded with human β2m and HLA-A*02:01 ECD-BSP to form PSA146/HLA-A*02:01 complex monomer. Folded peptide/HLA-A*02:01 monomers were concentrated by ultrafiltration and further purified through size-exclusion chromatography. HiPrep 26/60 Sephacryl S-300 HR was equilibrated with 1.5 column volumes of Hyclone Dulbecco's Phosphate Buffered Saline solution (Thermo Scientific, Cat No. SH3002802). The unpurified sample was loaded and eluted with 1 column volume. The first peak, corresponding to misfolded aggregates, eluted at approximately 111 mL, and the peak corresponding to the properly folded MHC complex was observed at 212 mL (FIG. 1). Peptide/HLA-A*02:01 monomers were biotinylated via BirA-mediated enzymatic reaction and subsequently purified by high-resolution anion-exchange chromatography. Biotinylated peptide/HLA-A*02:01 monomers were stored in PBS at −80° C.

SDS-PAGE of the purified PSA peptide/MHC complex can be performed to determine protein purity. For example, 1 μg of the protein complex is mixed with 2.5 μL of the NuPAGE LDS Sample Buffer (Life Technologies, NP0008) and brought up to 10 μL with deionized water. The sample is heated at 70° C. for 10 minutes, and then loaded onto the gel. Gel electrophoresis is performed at 180V for 1 hour.

### Example 2. Selection and Characterization of scFv Specific for PSA146/HLA-A*02:01 Complexes

A collection of human scFv antibody phage display libraries (diversity=10×10¹⁰) constructed by Eureka Therapeutics was used for the selection of human mAbs specific to PSA 146/HLA-A*02:01, 24 fully human phage scFv libraries were used to pan against PSA146/HLA-A*02:01 complex. In order to reduce the conformational change of MHCI complex introduced by immobilizing the protein complex onto plastic surfaces, solution panning and cell panning were used in place of conventional plate panning. In solution panning, biotinylated antigens were first mixed with the human scFv phage library after extended washing with PBS buffer, and then antigen-scFv antibody phage complexes were pulled down by streptavidin-conjugated Dynabeads M-280 through a magnetic rack. The bound clones were then eluted and used to infect E. coli XL1-Blue cells. In cell panning. T2 cells loaded with PSA 146 peptide were first mixed with the human scFv phage library. T2 cells are a TAP-deficient. HLA-A*02:01⁺ lymphoblast cell line. To load peptide, T2 cells were pulsed with peptides (50 ug/ml) in serum-free RPM11640 medium in the presence of 20 μg/ml β2M overnight. After extended washing with PBS, peptide-loaded T2 cells with bound scFv antibody phage were spun down. The bound clones were then eluted and used to infect E. coli XL1-Blue cells. The phage clones expressed in bacteria were then purified. The panning was performed for 3-4 rounds with either solution panning, cell panning or a combination of solution and cell panning to enrich for scFv phage clones that bound PSA146/HLA-A*02:01 specifically.

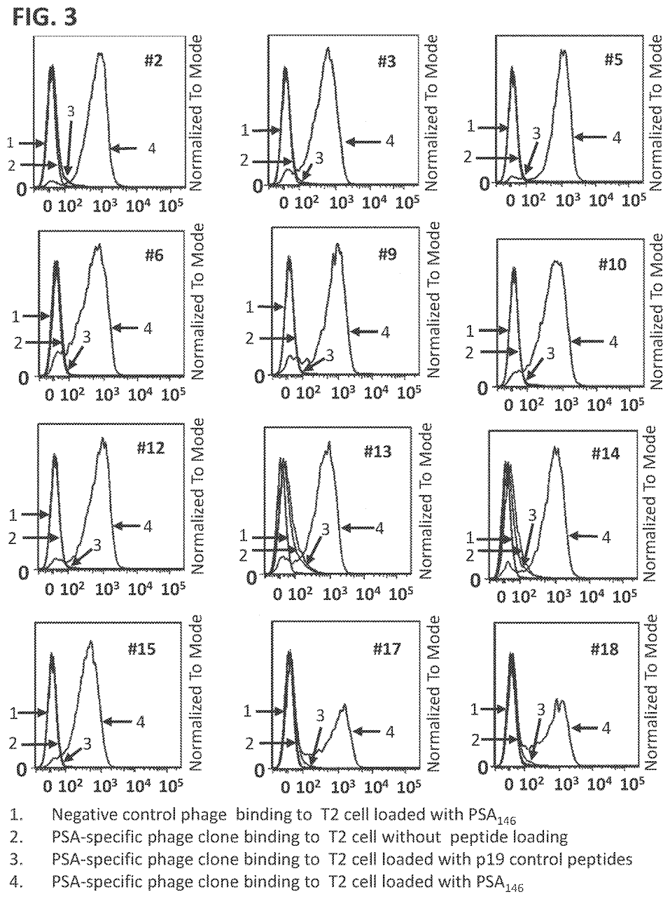

Streptavidin ELISA plates were coated with biotinylated PSA 146/HLA-A*02:01 complex monomer (Bio-PSA 146 mutant) or biotinylated control peptide mixture P19/HLA-A*02:01 monomer (Bio-P19). Individual phage clones from enriched phage display panning pools against PSA146/HLA-A*02:01 complex were incubated in the coated plates. Binding of the phage clones was detected by HRP-conjugated anti-M13 antibodies and developed using HRP substrate. The absorbance was read at 450 nm. 115 positive clones were identified through ELISA screening of 1033 phage clones enriched from phage panning. FIG. 2 provides an example of phage clone binding to biotinylated PSA 146/HLA-A*02:01 monomer in an ELISA assay. 16 unique clones (clone # s 2, 3, 5, 6, 8, 9, 10, 12, 13, 14, 15, 17, and 18; see Table 9) were identified by DNA sequencing of the 115 ELISA-positive phage clones (clone #5, for example, had the heavy chain variable region nucleotide sequence of SEQ ID NO: 179 and the light chain variable region nucleotide sequence of SEQ ID NO: 180). Positive clones were determined by standard ELISA against biotinylated PSA 146/HLA-A*02:01 complex monomer. Then, unique antibody clones were identified through DNA sequencing of ELISA-positive clones. Specific and unique clones were further tested for their binding to HLA-A*02:01/peptide complexes on live cell surface by flow cytometry (FACS analysis) using PSA 146-loaded live T2 cells. T2 cells loaded with different peptides and β2M were first stained with purified scFv phage clones, followed by staining with a mouse anti-M13 mAb, and finally R-PE conjugated horse anti-mouse IgG from Vector Labs. Each step of the staining was performed for between 30-60 minutes on ice and the cells were washed twice between stainings. All 16 clones recognized PSA146-loaded T2 cells specifically. FIG. 3 provides examples of PSA146/HLA-A*02:01 specific phage clone binding to peptide-loaded T2 cells through FACS. The phage clones specifically bound to PSA146-loaded T2 cells, and did not recognize T2 cells loaded with control peptide mixture (P19) or T2 cells with no peptide loaded. A negative control scFv phage clone showed no binding to PSA146-loaded T2 cells, demonstrating the antibody-specific binding of the PSA-positive scFv antibody phage clones to PSA146-loaded T2 cells. The negative control scFv phage clone presents a human antibody previously found not to bind any cell-surface human antigens.

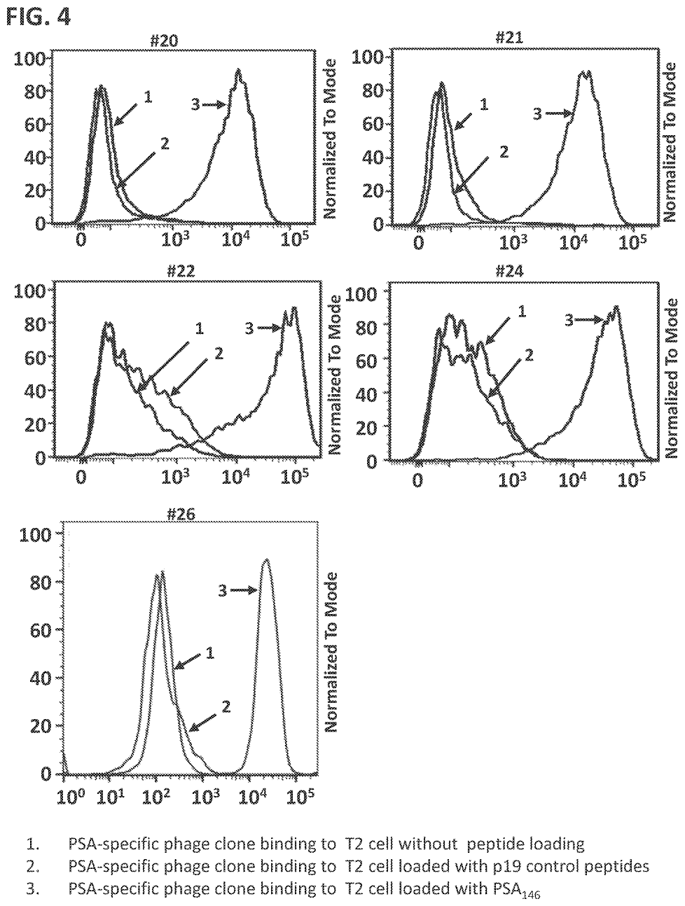

To identify additional PSA146/HLA-A*02:01 specific phage clones. 28 phage scFv libraries were used to pan against PSA 146/HLA-A*02:01 complex as described above. 122 positive clones were identified through ELISA screening of 1414 phage clones enriched from phage panning. 13 unique clones were identified by DNA sequencing of the 122 ELISA-positive phage clones, 11 of which were new clones not identified in the previous screen. The new clones were further tested for their binding to HLA-A*02:01/peptide complexes on live cell surface by FACS analysis using PSA146-loaded live T2 cells as described above. 5 new clones (clone # s 20, 21, 22, 24, and 26; see Table 9) recognized PSA146-loaded T2 cells specifically (FIG. 4).

TABLE 9

| | Light chain variable | Heavy chain variable |
|---|---|---|
| Clone | region (SEQ ID NO) | region (SEQ ID NO) |
| #2 | 27 | 14 |
| #3 | 28 | 15 |
| #5 | 29 | 16 |
| #6 | 30 | 17 |
| #8 | 31 | 18 |
| #9 | 32 | 19 |
| #10 | 33 | 20 |
| #12 | 34 | 21 |
| #13 | 35 | 22 |
| #14 | 36 | 23 |
| #15 | 37 | 24 |
| #17 | 38 | 25 |
| #18 | 39 | 26 |
| #20 | 150 | 145 |
| #21 | 151 | 146 |
| #22 | 152 | 147 |
| #24 | 153 | 148 |
| #26 | 154 | 149 |

### Example 3. Characterization of FACS-Positive PSA-Specific Phage Clones

### Antibody Binding Specificity Evaluation Against Endogenous Peptides

On average, each nucleated cell in the human body expresses about half a million different peptide/MHC Class I complexes. In order to develop anti-peptide/MHCI-complex antibodies into anti-cancer drugs with high specificity and therapeutic index, it is essential for the antibodies to specifically recognize the target peptide/MHCI complex, but not the MHCI molecule itself, or MHCI molecules bound to other peptides presented on cell surfaces. For the current study, the relevant MHCI molecule is HLA-A*02:01. During the early stages of our phage panning and screening, we eliminated antibodies that bound to the HLA-A*02:01 molecule alone or antibodies recognizing non-PSA 146 peptide (the P19 peptide mixture)/HLA-A*02:01 complexes (see, for example, FIG. 2). The top phage clones were then screened against 19 endogenous HLA-A*02:01 peptides (p19, SEQ ID NOs: 125-143) in FACS binding assays. The endogenous peptides were derived from proteins normally expressed in multiple types of nucleated human cells, such as globin alpha chain, beta chain, nuclear protein p68, and the like. As shown in FIG. 3, the PSA146/HLA-A*02:01-specific antibody phage clones bound PSA146/HLA-A*02:01 complex, but not HLA-A*02:01 complexes folded with endogenous peptides. We conclude that the identified antibodies are specific to PSA146/HLA-A*02:01 complexes, and do not recognize HLA-A*02:01 molecules bound to other HLA-A*02:01-restricted peptides.

### Epitope Mapping by Alanine Walking

To investigate with precision the epitope for the mAb recognition, PSA146 peptides with alanine substitutions at positions 1, 3, 4, 5, 6, 7 and 8 are pulsed onto T2 cells. Antibody phage clones are then tested for binding to these peptide-loaded T2 cells by FACS analysis. KO7 helper phage is included as a negative control to show that the phage alone without scFv presentation on the phage particle surface does not bind to any of the peptide-loaded T2 cell groups. BB7.2 antibody, which recognizes the HLA-A02 alpha chain, is included to show that the alanine-substituted peptides are still able to bind HLA-A*02:01 molecules on the T2 cell surface, as peptide binding to MHC complex stabilizes cell-surface MHC complexes. Controls include T2 cells without peptide loading (no peptide), antibody BB7.2, and KO7 helper phage.

### Example 4. Engineering Bispecific Antibodies

Bispecific antibodies (BsAbs) are generated using scFv sequences of the PSA146/HLA-A*02:01-specific phage clones. The BsAbs are single-chain bispecific antibodies comprising the scFv sequence of an PSA 146/HLA-A*02:01-specific phage clone at the N-terminal end and an anti-human CD3ε mouse monoclonal scFv at the C-terminal end (Brischwein, K. et al., Molecular Immunology 43:1129-1143, 2006). DNA fragments coding for the PSA146 scFv and the anti-human CD3ε scFv are synthesized, for example, by Genewiz and subcloned into Eureka's mammalian expression vector pGSN-Hyg using standard DNA techniques. A hexhistamine tag may be inserted at the C-terminal end for antibody purification and detection. Chinese hamster ovary (CHO) cells are transfected with the BsAb expression vector, and then cultured for 7 days for BsAb antibody production. CHO cell supernatants containing secreted PSA BsAb molecules are then collected. BsAbs are purified using, for example. HisTrap HP column (GE healthcare) by FPLC AKTA system. Briefly. CHO cell culture is clarified and loaded onto the column with low imidazole concentration (20 mM), and then an isocratic high imidazole concentration elution buffer (500 mM) is used to elute the bound BsAb proteins. Purity and molecular weight of the purified PSA BsAbs are determined under reducing conditions by gel electrophoresis. For example, 4 μg of the protein is mixed with 2.5 μL of the NuPAGE LDS Sample Buffer (Life Technologies, NP0008) and brought up to 10 μL with deionized water. The sample is heated at 70° C. for 10 minutes, and then loaded onto the gel. Gel electrophoresis is performed at 180V for 1 hour. ˜50 KD bands are observed as the major bands on the gel.

Antibody aggregation can be assessed by size-exclusion chromatography (SEC). For example, 50 μL of the sample is injected into a SEC column (for example Agilent, BioSEC-3.300A. 4.6×300 mm) while flowing a buffer consisting of Dulbecco's Phosphate Buffered Saline (Fisher Scientific, SH30028.FS) and 0.2M arginine adjusted to pH 7.0. BsAbs with high molecular weight aggregation less than 10% are selected for further characterization.

BsAbs were generated as described above, using scFvs from clones # s 2, 3, 5, 6, 9, 10, 12, 13, 14, 15, 17, and 18.

### Example 5. Characterization of PSA BsAb Antibodies

### Binding Affinity of PSA BsAb Antibodies

The binding affinity of PSA BsAb antibodies to recombinant PSA146/HLA-A*02:01 complex is measured, for example, by Surface Plasma Resonance (BiaCore). The binding parameters between the PSA146 BsAb and the PSA146 wild type peptide/HLA-A*02:01 complex are measured using a streptavidin chip with biotin capture kit by Biacore X100 (GE Healthcare) according to the manufacturer's protocol for multi-cycle kinetics measurement. All the proteins used in the assay are diluted using HBS-E buffer. In brief, 10 ug/mL of the biotin-labelled PSA146 wild type peptide/HLA-A*02:01 complex is immobilized onto a streptavidin sensor chip. Binding towards the PSA146 BsAbs is analyzed at, for example, 1.875, 3.75, 7.5, 15, and 30 μg/mL, each run consisting of a 3 minute association and 3 minute dissociation at 30 μL/min. At the end of cycle, the surface is regenerated using the regeneration buffer from the biotin capture kit. Following the kinetics measurement, the surface is regenerated using the regeneration solution from the kit. The data are analyzed using 1:1 binding site mode with the BiaCore X-100 evaluation software. The binding parameters (association on rate constant k_{d}), dissociation constant k_{d}, and equilibrium dissociation constant K_{d}) are then calculated.

### Cross-Reactivity of PSA BsAb Antibodies Against Multiple HLA-A02 Alleles

Human MHCI molecules consist of 6 class isoforms, HLA-A, -B, -C, -E, -F and G. The HLA-A, -B and -C heavy chain genes are highly polymorphic. For each isoform, the HLA genes are further grouped according to the similarity of heavy chain sequences. For example, HLA-A is divided into different alleles such as HLA-A01, -A02, -A03, etc. For the HLA-A02 allele, there are multiple subtypes, such as HLA-A*02:01, A*02:02, etc. Between the different subtypes of HLA-A02 group, the sequence differences are limited to only several amino acids. So in many cases, peptides that bind to HLA-A*02:01 molecule can also form complexes with multiple subtypes of the HLA-A02 allele. As shown in Table 10 (http://www.allelefrequencies.net/), although HLA-A*02:01 is the dominant HLA-A02 subtype among Caucasian populations, in Asia. A*02:05, A*02:06, A*02:07 and A*02:11 are also common HLA-A02 subtypes. The ability of PSA antibodies to recognize not only PSA peptide in the context of HLA-A*02:01, but also other subtypes of HLA-A02, will greatly broaden the patient population that might be able to benefit from PSA antibody drug treatment. Recombinant PSA 146/MHCI complexes with other subtypes of the HLA-A02 allele are generated and tested for binding affinity with the PSA146/HLA-A*02:01-specific antibodies. Binding affinity is determined, for example, using a ForteBio Octet QK. Briefly, 5 μg/mL biotinylated HLA-A*02 MHC complex having varying subtypes is loaded onto a streptavidin biosensor. After washing off excess antigen, BsAb antibodies are tested at, for example, 10 μg/mL for association and dissociation. Binding parameters are calculated using a 1:1 binding site, partial fit model.

TABLE 10

| | | | | | | sub- | | |
|---|---|---|---|---|---|---|---|---|
| | | | | | north | saharan | | |
| | australia | china | europe | india | africa | africa | taiwan | us |
| A*02:01 | 97.8% | 39.5% | 94.0% | 53.9% | 73.3% | 56.3% | 35.1% | 79.4% |
| A*02:02 | 0.0% | 0.1% | 0.3% | 0.9% | 9.7% | 24.1% | 0.0% | 3.6% |
| A*02:03 | 0.0% | 15.3% | 0.2% | 4.9% | 0.0% | 0.4% | 19.3% | 2.2% |
| A*02:04 | 0.0% | 0.1% | 0.0% | 0.3% | 2.6% | 0.4% | 0.0% | 0.2% |
| A*02:05 | 1.1% | 0.9% | 3.2% | 5.8% | 13.8% | 15.9% | 0.1% | 4.5% |
| A*02:06 | 0.0% | 16.0% | 0.9% | 10.6% | 0.0% | 0.7% | 12.8% | 5.5% |
| A*02:07 | 1.1% | 26.1% | 0.4% | 0.4% | 0.0% | 0.0% | 32.7% | 2.4% |
| A*02:08 | 0.0% | 0.0% | 0.0% | 0.0% | 0.0% | 0.0% | 0.0% | 0.0% |
| A*02:09 | 0.0% | 0.0% | 0.0% | 0.0% | 0.0% | 0.0% | 0.0% | 0.0% |
| A*02:10 | 0.0% | 1.1% | 0.0% | 0.0% | 0.0% | 0.2% | 0.0% | 0.1% |
| A*02:11 | 0.0% | 0.1% | 0.1% | 22.3% | 0.0% | 1.5% | 0.0% | 1.7% |
| other A02 subtypes | 0.0% | 0.7% | 0.8% | 0.9% | 0.5% | 0.5% | 0.0% | 0.6% |
| (A*02:12-A*02:93) | | | | | | | | |
| | 100.0% | 100.0% | 100.0% | 100.0% | 100.0% | 100.0% | 100.0% | 100.0% |

T-Cell Killing Assay with Cancer Cell Lines

Tumor cytotoxicity is assayed, for example, by LDH Cytotoxicity Assay (Promega). Human T cells are activated and expanded with, for example, CD3/CD28 Dynabeads (Invitrogen) according to manufacturer's protocol. Activated T cells (ATC) are cultured and maintained in RPMI1640 medium with 10% FBS plus 30 U/ml IL-2, and used at day 7-14. Activated T cells (Effector cells) and Target cells are co-cultured at a 5:1 ratio with different concentrations of BsAbs for 16 hours. Cytotoxicities are then determined by measuring LDH activities in culture supernatants.

Target cells may include cancer cell lines that are PSA⁺/HLA-A*02:01⁺, PSA⁻/HLA-A*02:01⁺, PSA⁺/HLA-A*02:01⁻, and PSA/HLA-A*02:01⁻, such as prostate cancer cell lines LNCaP (ATCC CRL-1740; HLA-A2⁺, PSA⁺), MDA PCa 2b (ATCC CRL-2422; HLA-A2⁻, PSA⁺). VCaP (ATCC CRL-2876; HLA-A2⁻. PSA⁻), 22Rv1 (ATCC CRL-2505; HLA-A2⁻, PSA⁻), and PC3 (ATCC CRL-1435; HLA-A2⁻, PSA⁻); breast cancer cell line MDA-MB-231 (ATCC HTB-26; HLA-A2⁺, PSA⁻); and liver cancer cell line SK-Hep1 (ATCC HTB-52; HLA-A2⁺, PSA⁻). As controls, MDA-MB-231-MiniG (HLA-A2⁺. PSA⁺) and SK-HEP-1-MiniG (HLA-A2⁺. PSA⁺) are generated by transducing MDA-MB-231 and SK-HEP-1 with a PSA peptide expressing minigene cassette, which results in a high level of cell surface expression of PSA 146/HLA-A*02:01 complex.

T-Cell Killing Assay with Peptide-Pulsed T2 Cells

Peptide/MHC complex-specific cytotoxicity is assayed, for example, by LDH Cytotoxicity Assay (Promega). For example, human T cells are activated and expanded with CD3/CD28 Dynabeads (Invitrogen) according to manufacturer's protocol. Activated T cells (ATC) are cultured and maintained in RPMI1640 medium with 10% FBS plus 30 U/ml IL-2, and used at day 7-14. Activated T cells (effector cells) and target peptide-loaded T2 cells are co-cultured at a 5:1 ratio with 1 μg/ml or 0.2 μg/ml of BsAh for 16 hours. Peptide-loaded T2 cells are prepared by incubating T2 cells overnight with 50 μg/ml of either the target PSA 146-154 peptide (KLQCVDLHV, SEQ ID NO: 4) or negative control peptide, such as AFP158 peptide (FMNKFIYEI, SEQ ID NO: 144). A negative control BsAb, such as AFP158/HLA-A*02:01 specific BsAb, is optionally included. Cytotoxicities are determined by measuring LDH activities in culture supernatants.

### Example 6. Generation of PSA146/HLA-A*02:01 Specific Chimeric Antigen Receptor-Presenting T Cells (CAR-T)

Chimeric antigen receptor therapy (CAR-T therapy) is a new form of targeted immunotherapy. It merges the exquisite targeting specificity of monoclonal antibodies with the potent cytotoxicity and long-term persistence provided by cytotoxic T cells. This technology enables T cells to acquire long-term novel antigenic specificity independent of the endogenous TCR. Clinical trials have shown clinically significant antitumor activity of CAR-T therapy in neuroblastoma (Louis C. U. et al., Blood 118(23):6050-6056), B-ALL (Maude S. L. et al., N. Engl. J. Med. 371(16): 1507-1517, 2014). CLL (Brentjens R. J. et al., Blood 118(18):4817-4828, 2011), and B cell lymphoma (Kochenderfer J. N. et al., Blood 116(20):4099-4102, 2010). In one study, a 90% complete remission rate in 30 patients with B-ALL treated with CD19-CAR T therapy was reported (Maude S. L. et al., supra).

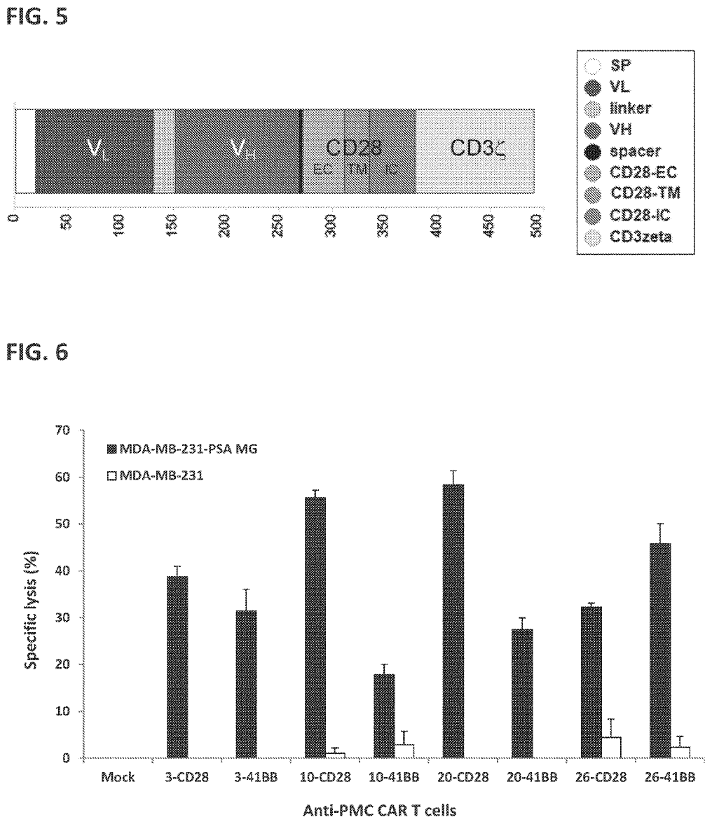

To further explore the potency of the PSA146/HLA-A*02:01 specific antibodies, anti-PMC scFv expressing CARs (anti-PMC CARs) are constructed and transduced into T cells. For example, PSA 46/HLA-A*02:01 specific CARs are constructed using a lentiviral CAR expression vector. Anti-PSA146/HLA-A*02:01 scFvs are grafted onto a second generation CAR (Mackall C. L. et al., Nat. Rev. Clin. Oncol. 11(12):693-703, 2014) with a CD28 or 4-1BB signaling domain and TCRζ engineered in cis to provide intracellular T cell stimulation signals and to activate T cells. FIG. 5 provides a schematic illustration of an exemplary anti-PMC CAR construct with a CD28 co-stimulatory signaling domain.

Anti-PMC CARs were constructed as described above using scFvs from clone # s 2, 3, 5, 6, 9, 10, 12, 13, 14, 15, 17, 18, 20, and 26. The anti-PMC CARs contained, in order from N-terminus to C-terminus, the light chain and heavy chain variable regions from a PMC-specific phage clone linked by a peptide linker (SEQ ID NO: 176), fused to either a 4-1BB/CD3ζ CAR sequence (SEQ ID NO: 178; for all of the clones mentioned in this paragraph, indicated as “clone #”-41BB) or a CD28/CD3ζ CAR sequence (SEQ ID NO: 177; for clones 3, 10, 20, and 26, indicated as “clone #”-CD28). For example, CAR 3-CD28 contained the scFv from clone 3 fused to the CD28/CD3ζ CAR sequence (SEQ ID NO: 177).

### Example 7. Characterization of PSA CAR-T Cells

### In Vitro Cytotoxicity Study of PSA CAR-T Cells

Lentiviruses containing PSA146/HLA-A*02:01 specific chimeric antigen receptors are produced, for example, by transfection of 293T cells with CAR vectors. Human T-cells are used for transduction after 2-day stimulation with CD3/CD28 beads (Dynabeads®, Invitrogen) in the presence of interleukin-2 at 30 U/ml. Concentrated lentiviruses are applied to T-cells in Retronectin (Takara) coated 6-well plates for 72 hours. Transduction efficiency is assessed by FACS using biotinylated PSA tetramer and PE-conjugated streptavidin. Repeat FACS analyses are done at 72 hours and every 3-4 days thereafter.

Functional assessment of the transduced T cells (anti-PMC CAR-T cells) is performed using LDH Cytotoxicity Assay. Effector-to-target ratios used include, for example, 5:1 and 10:1. The target cell lines may include, for example, prostate cancer cell lines LNCaP (ATCC CRL-1740; HLA-A2⁺. PSA⁺), MDA PCa 2b (ATCC CRL-2422; HLA-A2⁻, PSA⁺), VCaP (ATCC CRL-2876; HLA-A2⁻, PSA⁻). 22Rv1 (ATCC CRL-2505; HLA-A2⁻. PSA⁻), and PC3 (ATCC CRL-1435; HLA-A2⁻, PSA3⁻); breast cancer cell line MDA-MB-231 (ATCC HTB-26; HLA-A2⁺, PSA⁻); and liver cancer cell line SK-Hep1 (ATCC HTB-52; HLA-A2⁺, PSA⁻). As controls, MDA-MB-231-MiniG and SK-HEP-1-MiniG are generated by transducing MDA-MB-231 and SK-HEP-1 with a PSA peptide expressing minigene cassette, which results in a high level of cell surface expression of PSA146/HLA-A*02:01 complex. The specificity and efficiency of the anti-PMC CAR expressing T cells to kill the target-positive cancer cells is determined.

An in vitro cytotoxicity assay was carried out as described above using target cell lines MDA-MB-231 (HLA-A2⁺, PSA⁻) and MDA-MB-231 MiniG (HLA-A2⁺, PSA⁺) and T cells transduced with the following anti-PMC CARs: 3-CD28, 3-41BB, 10-CD28, 10-41BB, 20-CD28, 20-41BB, 26-CD28, and 26-41BB. As shown in FIG. 6, all of the anti-PMC CAR T cells tested killed the target-positive MDA-MD-231 MiniG cells in a specific and highly efficient manner, with target-negative MDA-MD-231 cells being poorly recognized. These results demonstrate that the anti-PMC CAR T cells are highly specific to PSA-expressing cells and do not mediate non-specific killing.

### Example 8. Generation and Characterization of the Full-Length IgG1 PSA Antibodies

Full-length human IgG1 of the selected phage clones are produced, for example, in HEK293 and Chinese hamster ovary (CHO) cell lines, as described (Tomimatsu K. et al., Biosci. Biotechnol. Biochem. 73(7):1465-1469, 2009). In brief, antibody variable regions are subcloned into mammalian expression vectors, with matching human lambda or kappa light chain constant region and human IgG1 constant region sequences. Applying the same cloning strategy, chimeric PSA full-length antibodies with mouse IgG11 heavy chain and light chain constant regions are generated. Molecular weight of the purified full length IgG antibodies is measured under both reducing and non-reducing conditions by electrophoresis. SDS-PAGE of purified PSA mouse chimeric IgG1 antibodies is performed to determine protein purity. In brief, 2 μg of the protein is mixed with 2.5 μL of NuPAGE LDS Sample Buffer (Life Technologies, NP0008) and brought up to 10 μL with deionized water. The sample is heated at 70° C. for 10 minutes, and then loaded onto the gel. Gel electrophoresis is performed at 180V for 1 hour.

PSA chimeric IgG1 antibody is tested for binding towards PSA presenting SK-HEP-1 cells by flow cytometry. SK-HEP-1 is an HLA-A*02:01 positive and PSA negative cell line. A PSA minigene cassette is transfected into SK-HEP-1 cells to generate the PSA-presenting SK-HEP-1-miniG cells. 10 μg/mL of antibody is added to cells on ice for 1 hour. After washing, R-PE conjugated anti-mouse IgG(H+L) (Vector Labs # EI-2007) is added to detect antibody binding. Binding affinity of the mouse chimeric IgG1 PSA antibodies is determined by ForteBio Octet QK. 5 μg/mL biotinylated PSA peptide/HLA-A*02:01 complex is loaded onto a streptavidin biosensor. After washing off excess antigen, mouse chimeric full-length antibodies are tested at 10 μg/mL for association and dissociation kinetics. Binding parameters are calculated using a 1:1 binding site, partial fit model.

PSA-specific and negative control (such as ET901) mouse chimeric IgG1 are tested for binding towards PSA 146/HLA-A*02:01, PSA recombinant protein and free PSA peptide in an ELISA assay. Antibodies are tested, for example, at 3× serial dilution, starting from 100 ng/mL for a total of 8 concentrations. Biotinlyated PSA146/HLA-A*02:01 MHC is coated onto streptavidin plates at 2 μg/mL, PSA protein is coated at 2 μg/mL and PSA peptide is coated at 40 ng/mL. The ability of full-length anti-PSA 146/HLA-A*02:01 antibodies to recognize the PSA peptide only in the context of HLA-A02, and not bind recombinant PSA protein or free PSA peptide is determined.

### Example 9. In Vivo Efficacy Studies

### PSA CAR-T Cell Treatment in Mice

HLA-A02⁺/PSA⁺ cancer cell line sub-cutaneous (s.c.) xenograft models are generated in SCID-beige (no functional T-, B-, NK-cells) mice. Animals are randomized when average s.c. tumor volume reaches 200 mm³. 24 hours prior to CART administration, animals are treated (via intraperitoneal route) with 60 mg/kg cyclophosphamide. Mice are divided into 4 groups (n=8-10 mice/group) that receive one of the following: (i) no treatment (ii) 10⁷ mock transduced CAR T cells, ix/week for 4 weeks (iii) 10⁷ anti-PMC CAR T cells. 1×/week for 4 weeks, or (iv) 2×10⁶ anti-PMC CAR T cells, ix/week for 4 weeks. The animals in each group are monitored for tumor volume, adverse response, human cytokine profile, histopathology of tumor for human CD3⁺ cells in tumor and organs for CAR T cell infiltration, serum PSA, body weight and general health condition (eating, walking, daily activities).

Sequence Listing

SEQ ID NO: 1, PSA protein
MWVPVVFLTLSVTWIGAAPLILSRIVGGWECEKHSQPWQVLVASRGRAVC
GGVLVHPQWVLTAAHCIRNKSVILLGRHSLFHPEDTGQVFQVSHSFPHPL
YDMSLLKNRFLRPGDDSSHDLMLLRLSEPAELTDAVKVMDLPTQEPALGT
TCYASGWGSIEPEEFLTPKKLQCVDLHVISNDVCAQVHPQKVTKFMLCAG
RWTGGKSTCSGDSGGPLVCNGVLQGITSWGSEPCALPERPSLYTKVVHYR
KWIKDTIVANP
SEQ ID NO: 2, PSA CDS
ATGTGGGTCCCGGTTGTCTTCCTCACCCTGTCCGTGACGTGGATTGGTGC
TGCACCCCTCATCCTGTCTCGGATTGTGGGAGGCTGGGAGTGCGAGAAGC
ATTCCCAACCCTGGCAGGTGCTTGTGGCCTCTCGTGGCAGGGCAGTCTGC
GGCGGTGTTCTGGTGCACCCCCAGTGGGTCCTCACAGCTGCCCACTGCAT
CAGGAACAAAAGCGTGATCTTGCTGGGTCGGCACAGCCTGTTTCATCCTG
AAGACACAGGCCAGGTATTTCAGGTCAGCCACAGCTTCCCACACCCGCTC
TACGATATGAGCCTCCTGAAGAATCGATTCCTCAGGCCAGGTGATGACTC
CAGCCACGACCTCATGCTGCTCCGCCTGTCAGAGCCTGCCGAGCTCACGG
ATGCTGTGAAGGTCATGGACCTGCCCACCCAGGAGCCAGCACTGGGGACC
ACCTGCTACGCCTCAGGCTGGGGCAGCATTGAACCAGAGGAGTTCTTGAC
CCCAAAGAAACTTCAGTGTGTGGACCTCCATGTTATTTCCAATGACGTGT
GTGCGCAAGTTCACCCTCAGAAGGTGACCAAGTTCATGCTGTGTGCTGGA
CGCTGGACAGGGGGCAAAAGCACCTGCTCGGGTGATTCTGGGGGCCCACT
TGTCTGTAATGGTGTGCTTCAAGGTATCACGTCATGGGGCAGTGAACCAT
GTGCCCTGCCCGAAAGGCCTTCCCTGTACACCAAGGTGGTGCATTACCGG
AAGTGGATCAAGGACACCATCGTGGCCAACCCCTGA
SEQ ID NO: 3, PSA 108-117
LTDAVKVMDL
SEQ ID NO: 4, PSA 146-154
KLQCVDLHV
SEQ ID NO: 5, PSA 154-163
VISNDVCAQV
SEQ ID NO: 6, PSA 141-150
FLTPKKTQCV
SEQ ID NO: 7, PSA 146-154 A1
ALQCVDLHV
SEQ ID NO: 8, PSA 146-154 A3
KLACVDLHV
SEQ ID NO: 9, PSA 146-154 A4
KLQAVDLHV
SEQ ID NO: 10, PSA 146-154 A5
KLQCADLHV
SEQ ID NO: 11, PSA 146-154 A6
KLQCVALHV
SEQ ID NO: 12, PSA 146-154 A7
KLQCVDAHV
SEQ ID NO: 13, PSA 146-154 A8
KLQCVDLAV
SEQ ID NO: 14, HCVR 2
EVQLVQSGAEVKRPGSSVKVSCKASGGTFSSYAINWVRRAPGQGLEWMGK
IIPIPGITNYAQKFQDRVTFTADTSTNIAYMELSSLRSEDTAMYYCARSY
KWGSSLVDAWGQGTLVTVSS
SEQ ID NO: 15, HCVR 3
EVQLVESGGGLVQPGGSLRLSCAASGFTFSSYAMSWVRQAPGKGLEWVSA
ISGSGGSTYYADSVKGRETISRDNSKNTLYLQMNSLRAEDTAVYYCARNY
YSQYWMMDLWGQGTLVTVSS
SEQ ID NO: 16, HCVR 5
QVQLVQSGAEVKKPGDSVKVSCKPSGYNFLNYGINWVRQAPGQGLEWMGW
ISTYTGNTNYAQKLQGRVTFTTDTSTSTAYMEMRSLRSDDTAVYYCARSS
EYYTWDHWGQGTLVTVSS
SEQ ID NO: 17, HCVR 6
EVQLVQSGAEVKKPGASVKVSCKASGYTFTGYYIHWVRQAPGQGLEWMGG
FDPEDGETIYAQKFQDRVTMTADTSTDTAYMELSSLRSEDTAVYYCARYG
FDYWGQGTLVTVSS
SEQ ID NO: 18, HCVR 8
QVQLQQWGAGLLKPSETLSLTCAVKGGSFSDYYWSWIRQPPGKGLEWIGE
INHSGGTNYNPSLKSRVTISVDTSKNQFSLKLLSVTAADTAVYYCARYNE
YGSGYDKWGQGTLVTVSS
SEQ ID NO: 19, HCVR 9
QVQLVQSGAEVKKPGASVKVSCKASGYTFTSYGISWVRQAPGQGLEWMGW
ISAYNGNTNYAQKLQGRVTMTTDTSTSTAYMELRSLRSDDTAVYYCARSS
QYYVWDSWGQGTLVTVSS
SEQ ID NO: 20, HCVR 10
QVQLVQSGAEVKKPGSSVKVSCKASGGTFSSYAISWVRQAPGQGLEWMGW
INPNSGGTNYAQKFQGRVTMTRDTSISTAYMELSRLRSDDTAVYYCARWS
YYYFQQFWSLDGWGQGTLVTVSS
SEQ ID NO: 21, HCVR 12
QMQLVQSGSEVKKPGASVKVSCKASGYTFTSYGISWVRQAPGQGLEWMGW
ISAYNGNTDYVQKLQGRVTMTTDTSTNTAYMELGSLGSDDTAVYYCARTN
YNKYDIWGQGTLVTVSS
SEQ ID NO: 22, HCVR 13
QVQLVQSGAEVKKPGASVKVSCKASGYTFTSYYMHWVRQAPGQGLEWMGG
FDPEDGETIYAQKFQGRVTMTEDTSTDTAYMGLSSLRSEDTAVYYCARYS
YDYWGQGTLVTVSS
SEQ ID NO: 23, HCVR 14
EVQLVQSGAEVKKPGASVKVSCKASGYTFTGYFMHWVRQAPGQGLEWMGG
FDPEDGETIYAQKFQGRVTMTEDTSTDTAYMELSSLRSEDTAVYYCARYS
YDLWGQGTLVTVSS
SEQ ID NO: 24, HCVR 15
QVQLVQSGAEVKKPGSSVKVSCKASGGTFSSYAISWVRQAPGQGLEWMGR
IIPILGIANYAQKFQGRVTITADKSTSTAYMELSSLRSEDTAVYYCARVS
QPVYGSSTYDIWGQGTLVTVSS
SEQ ID NO: 25, HCVR 17
QMQLVQSGAEVKKPGESLKISCKGSGYSFTSYWIGWVRQMPGKGLEWMGI
IYPGDSDTRYSPSFQGQVTISADKSISTAYLQWSSLKASDTAMYYCARLV
VPDAFDIWGQGTMVTVSS
SEQ ID NO: 26, HCVR 18
QVQLVQSGAEVKKPGESLKISCKGSGYSFTSYWIGWVRQMPGKGLEWMGI
IYPGDSDTRYSPSFQGQVTISADKSISTAYLQWSSLKASDTAMYYCARWG
SRGFLDAFDIWGQGTMVTVSS
SEQ ID NO: 27, LCVR 2
QSVLTQPPSASGTPGQRVTLSCSGSNSNIGSNTVNWYQQLPGTNPKLLIY
SNNQRPSGVPDRFSGSKSGTSASLAISGLQSEDEADYYCATWDDSLNGPV
FGGGTKLTVLG
SEQ ID NO: 28, LCVR 3
QSVLTQPPSVSGAPGQRVTISCTGSSSNFGAGYDVHWYQQLPGAAPKLLI
YGDTNRPSGVPDRFSGSKSGTSASLAITGLQAEDEADYYCQSYDTSLSGS
VFGGGTKETVLG
SEQ ID NO: 29, LCVR 5
QSVLTQPPSVSGAPGQRVTISCTGSSSNIGAGYDVHWYQQLPGTAPKLLI
YGNSNRPSGVPDRFSGSKSGTSASLAITGLQAEDEADYYCQSYDSSLSGW
VFGGGTKLTVLG
SEQ ID NO: 30, LCVR 6
QAVVTQEPSUIVSPGGTVTLTCASSTGAVTSGYYPNWFQLKPGQAPRALI
YTTGKKHSWAPARFSGSLLGGKAALTLSGVQPEDEAEYYCLLYSGGVWVF
GGGTKLTVLG
SEQ ID NO: 31, LCVR 8
QSVLTQPPSVSAAPGQKVTISCSGSSYNIGNNYVSWYQQLPGTAPKLLIY
DNNKRPSGIPDRFSGSKSGTSATLGITGLQTGDEAEYYCGTWESSLSAYV
FGTGTKVTVLG
SEQ ID NO: 32, LCVR 9
QSVVTQPPSVSGAPGQRVTISCTGSSSNFGAGYDVHWYQQLPGTAPKLLI
YGNSNRPSGVPDRFSGSKSGTSASLAITGLQAEDEADYYCQSYDSSLSGW
VFGGGTKLTVLG
SEQ ID NO: 33, LCVR 10
QAVLTQPPSASGTPGQRVTISCSGSSSNIGSNTVNWYQQLPGTAPKLLIY
SNNQRPSGVPDRFSGSKSGTSASLAISGLQSEDEADYYCAAWDDSLNGRW
VFGGGTKLTVLG
SEQ ID NO: 34, LCVR 12
QSVLTQPPSVSGAPGQRVTISCTGSSSNIGAGYDVHWYQQLPGTAPKLLI
YGNSNRPSGVPDRFSGSKSGTSASLAITGLQAEDEADYYCQSYDSSLSEV
FGTGTKVTVLG
SEQ ID NO: 35, LCVR 13
QAVVTQEPSLTVSPGGTVTLTCASSTGAVTSGYYPNWFQQKPGQAPRALI
YSTSNKHSWTPARFSGSLLGGKAALTLSGVQPEDEAEYYCLLYYGGAYVF
GTGTKVTVLG
SEQ ID NO: 36, LCVR 14
QTVVTQEPSLIVSPGGTVTLTCASSTGAVTSGYYPNWFQQKPGQAPRALI
YSTSNKHSWTPARFSGSLLGGKAALTLSGVQPEDEAEYYCLLYYGGAQWV
FGGGTKLTVLG
SEQ ID NO: 37, LCVR 15
QAVLTQPPSASGTPGQRVTISCSGRSSNLGSNSVNWYQQVPGTAPKLLIF
DNHQRPSGVPDRFSGSKSGTSASLAISGLRSEDETDYYCAAWDDSLNSVV
FGGGTKLTVLG
SEQ ID NO: 38, LCVR 17
QSVVTQPPSVSAAPGQKVTISCSGSSSNIGNNYVSWYQQLPGTAPKLLIY
DNYKRPSGIPDRFSGSKSGTSATLGITGLQTGDEADYYCGTWDSSLSAGV
FGGGTKLTVLG
SEQ ID NO: 39, LCVR 18
QSVVTQPPSVSAAPGQRVTISCSGSSSNIGNNYVSWYQQLPGAAPRLLIY
DNDKRPSGIPDRFSGSKSGTSATLGITGLQTGDEADYYCGTWDSSLSSGV
FGGGTKLTVLG
SEQ ID NO: 40, HC-CDR1 2
GGTFSSYA
SEQ ID NO: 41, HC-CDR1 3
GFTFSSYA
SEQ ID NO: 42, HC-CDR1 5
GYNFLNYG
SEQ ID NO: 43, HC-CDR1 6
GYTFTGYY
SEQ ID NO: 44, HC-CDR1 8
GGSFSDYY
SEQ ID NO: 45, HC-CDR1 9
GYTFTSYG
SEQ ID NO: 46, HC-CDR1 10
GGTFSSYA
SEQ ID NO: 47, HC-CDR1 12
GYTFTSYG
SEQ ID NO: 48, HC-CDR1 13
GYTFTSYY
SEQ ID NO: 49, HC-CDR1 14
GYTFTGYF
SEQ ID NO: 50, HC-CDR1 15
GGTFSSYA
SEQ ID NO: 51, HC-CDR1 17
GYSFTSYW
SEQ ID NO: 52, HC-CDR1 18
GYSFTSYW
SEQ ID NO: 53, HC-CDR2 2
IIPIPGIT
SEQ ID NO: 54, HC-CDR2 3
ISGSGGST
SEQ ID NO: 55, HC-CDR2 5
ISTYTGNT
SEQ ID NO: 56, HC-CDR2 6
FDPEDGET
SEQ ID NO: 57, HC-CDR2 8
INHSGGT
SEQ ID NO: 58, HC-CDR2 9
ISAYNGNT
SEQ ID NO: 59, HC-CDR2 10
INPNSGGT
SEQ ID NO: 60, HC-CDR2 12
ISAYNGNT
SEQ ID NO: 61, HC-CDR2 13
FDPEDGET
SEQ ID NO: 62, HC-CDR2 14
FDPEDGET
SEQ ID NO: 63, HC-CDR2 15
IIPILGIA
SEQ ID NO: 64, HC-CDR2 17
IYPGDSDT
SEQ ID NO: 65, HC-CDR2 18
IYPGDSDT
SEQ ID NO: 66, HC-CDR3 2
ARSYKWGSSLVDA
SEQ ID NO: 67, HC-CDR3 3
ARNYYSQYWMMDL
SEQ ID NO: 68, HC-CDR3 5
ARSSEYYTWDH
SEQ ID NO: 69, HC-CDR3 6
ARYGFDY
SEQ ID No: 70, HC-CDR3 8
ARYNEYGSGYDK
SEQ ID NO: 71, HC-CDR3 9
ARSSQYYVWDS
SEQ ID NO: 72, HC-CDR3 10
ARWSYYYFQQFWSLDG
SEQ ID NO: 73, HC-CDR3 12
ARTNYNKYDI
SEQ ID NO: 74, HC-CDR3 13
ARYSYDY
SEQ ID NO: 75, HC-CDR3 14
ARYSYDL
SEQ ID NO: 76, HC-CDR3 15
ARVSQPVYGSSTYDI
SEQ ID NO: 77, HC-CDR3 17
ARLVVPDAFDI
SEQ ID NO: 78, HC-CDR3 18
ARWGSRGFLDAFDI
SEQ ID NO: 79, LC-CDR1 2
NSNIGSNT
SEQ ID NO: 80, LC-CDR1 3
SSNFGAGYD
SEQ ID NO: 81, LC-CDR1 5
SSNIGAGYD
SEQ ID NO: 82, LC-CDR1 6
TGAVTSGYY
SEQ ID NO: 83, LC-CDR1 8
SYNIGNNY
SEQ ID NO: 84, LC-CDR1 9
SSNFGAGYD
SEQ ID NO: 85, LC-CDR1 10
SSNIGSNT
SEQ ID NO: 86, LC-CDR1 12
SSNIGAGYD
SEQ ID NO: 87, LC-CDR1 13
TGAVTSGYY
SEQ ID NO: 88, LC-CDR1 14
TGAVTSGYY
SEQ ID NO: 89, LC-CDR1 15
SSNLGSNS
SEQ ID NO: 90, LC-CDR1 17
SSNIGNNY
SEQ ID NO: 91, LC-CDR1 18
SSNIGNNY
SEQ ID NO: 92, LC-CDR2 2
SNN
SEQ ID NO: 93, LC-CDR2 3
GDT
SEQ ID NO: 94, LC-CDR2 5
GNS
SEQ ID NO: 95, LC-CDR2 6
TTG
SEQ ID NO: 96, LC-CDR2 8
DNN
SEQ ID NO: 97, LC-CDR2 9
GNS
SEQ ID NO: 98, LC-CDR2 10
SNN
SEQ ID NO: 99, LC-CDR2 12
GNS
SEQ ID NO: 100, LC-CDR2 13
STS
SEQ ID NO: 101, LC-CDR2 14
STS
SEQ ID NO: 102, LC-CDR2 15
DNH
SEQ ID NO: 103, LC-CDR2 17
DNY
SEQ ID NO: 104, LC-CDR2 18
DND
SEQ ID NO: 105, LC-CDR3 2
ATWDDSLNGPV
SEQ ID NO: 106, LC-CDR3 3
QSYDTSLSGSV
SEQ ID NO: 107, LC-CDR3 5
QSYDSSLSGWV
SEQ ID NO: 108, LC-CDR3 6
LLYSGGVWV
SEQ ID NO: 109, LC-CDR3 8
GTWESSLSAYV
SEQ ID NO: 110, LC-CDR3 9
QSYDSSLSGWV
SEQ ID NO: 111, LC-CDR3 10
AAWDDSLNGRWV
SEQ ID NO: 112, LC-CDR3 12
QSYDSSLSEV
SEQ ID NO: 113, LC-CDR3 13
LLYYGGAYV
SEQ ID NO: 114, LC-CDR3 14
LLYYGGAQWV
SEQ ID NO: 115, LC-CDR3 15
AAWDDSLNSVV
SEQ ID NO: 116, LC-CDR3 17
GTWDSSLSAGV
SEQ ID NO: 117, LC-CDR3 18
GTWDSSLSSGV
SEQ ID NO: 118, HC-CDR1 consensus
G-G/Y-S/T-F-S/T-S-Y-A/G
SEQ ID NO: 119,_HC-CDR2 consensus
I-X-P-X-X-G-X-T, wherein X is any amino acid
SEQ ID NO: 120, HC-CDR3 consensus
A-R-X-X-Y, wherein X is any amino acid
SEQ ID NO: 121, LC-CDR1 consensus
S/T-S-N-F/I-G-A/N/S-G/N-Y
SEQ ID NO: 122, LC-CDR3 consensus 1
A/G-A/T-W-D/S-S-L-N/S-A/G-X-V, wherein X is
any amino acid
SEQ ID NO: 123, LC-CDR3 consensus 2
L-L-Y-X-G-G, wherein X is any amino acid
SEQ ID NO: 124, LC-CDR3 consensus 3
Q-S-Y-D-S/T-S-L-S-G-X-V, wherein X is any
amino acid
SEQ ID NO: 125, A2E-1
LLDVPTAAV
SEQ ID NO: 126, A2E-2
TLWVDPYEV
SEQ ID NO: 127, A2E-3
FLLDHLKRV
SEQ ID NO: 128, A2E-4
LLLDVPTAAV
SEQ ID NO: 129, A2E-5
VLFRGGPRGLLAV
SEQ ID NO: 130, A2E-6
SLLPAIVEL
SEQ ID NO: 131, A2E-7
YLLPAIVHI
SEQ ID NO: 132, A2E-8
FLLPTGAEA
SEQ ID NO: 133, A2E-9
LLDPKLCYLL
SEQ ID NO: 134, A2E-10
SLPHFHHPET
SEQ ID NO: 135, A2E-11
MLLSVPLLLG
SEQ ID NO: 136, A2E-13
LLYDMVCGDIP
SEQ ID NO: 137, A2E-14
LLLDVPTAAVQ
SEQ ID NO: 138, A2E-15
LLLDVPTAAVQA
SEQ ID NO: 139, A2E-16
VLFRGGPRGLLAVA
SEQ ID NO: 140, A2E-17
MVDGTLLLL
SEQ ID NO: 141, A2E-19
YMAPEILMRS
SEQ ID NO: 142, A2E-21
FIYNADLMNC
SEQ ID NO: 143, A2E-23
KQYESVLMVSI
SEQ ID NO: 144, AFP158
FMNKFIYEI
SEQ ID NO: 145, HCVR 20
EVQLVQSGAEVKKPGESLKISCKGSGYSFTSYRIGWVRQMPGKGLEWMGI
IYPGDSDTRYSPSFQGQVTISADKSISTAYLQWSSLKASDTAMYYCARWG
LSWDGWGVTDYWGQGTLVTVSS
SEQ ID NO: 146, HCVR 21
QVQLVQSGAEVKKPGASVKVSCKASGYTFTSYYMHWVRQAPGKGLEWMGG
FDPEDGETIYAQKFQGRVTMTEDTSIDTAYMELSSIRSEDTAVYYCARYN
YDTWGQGTLVTVSS
SEQ ID NO: 147, HCVR 22
EVQLVQSGAEVKKPGESLKISCKGSGYSFTSYWIGWVRQMPGKGLEWMGR
IDPSDSYTNYSPSFQGHVTISADKSISTAYLQWSSLKASDTAMYYCARSF
GAGYDSWGQGTLVTVSS
SEQ ID NO: 148, HCVR 24
QVQLVQSGAEVKKPGASVKVSCKASGYTFTSYYMHWVRQAPGKGLEWMGG
FDPEDGETIYAQKFQGRVTMTEDTSTDTAYMELSSLRSEDTAVYYCARYP
WDHWGQGTLVTVSS
SEQ ID NO: 149, HCVR 26
QVQLVQSGAEVKKPGASVKVSCKTSGYTFTNYGISWVRQAPGQGLEWMGW
ISAYNGNTNYAQNLQGRVTMTTDTSTSTAYMELRSLRSDDTAVYYCARSS
YYGYLSDGWGQGTLVTVSS
SEQ ID NO: 150, LCVR 20
DIQLTQSPSSLSASVGDRVTITCRASQSISSYLNWYQQKPGKARKLLIYA
ASSLQSGVPSRFSGSGSGTDFTLTISSLQPEDFATYYCQQSYSTPFTFGP
GTKVDIKR
SEQ ID NO: 151, LCVR 21
QAVVTQEPSLTVSPGGTVTLTCASSTGTVTSTYYPNWFQQKPGQAPRALI
YSTSNRHSWTPARFSGSLLGGKAALTVSGVQPDDEAEYYCLVFYGGVWVF
GGGTKLTVLG
SEQ ID NO: 152, LCVR 22
QSALTQPASVSGSPGQSITISCTGTSSDVGGYNYVSWYQQHPGKAPKLMI
YDVSKRPSGVSNRFSGSKSGNTASLTISGLQAEDEADYYCSSYTSSSRYV
FGTGTKVTVLG
SEQ ID NO: 153, LCVR 24
QAVVTQEPSLTVSPGGTVTLTCASSTGAVTSGYYPNWFQQKPGQAPRPLI
YSTNNKHSWTPARFSGSLLGGKAALTLSGVQPEDEAEYYCLLYYGGQGVF
GGGTKLTVLG
SEQ ID NO: 154, LCVR 26
LPVLTQPPSASGTPGQRVTISCSGSSSNIGTNYVYWYQQLPGTAPKLLIY
SNNQRPSGVPDRFSGSKSGTSASLAISGLRSEDEADYYCAAWDDSLSGLY
VFGTGTKVTVLG
SEQ ID NO: 155, HC-CDR1 20
GYSFTSYR
SEQ ID NO: 156, HC-CDR1 26
GYTFTNYG
SEQ ID NO: 157, HC-CDR2 22
IDPSDSYT
SEQ ID NO: 158, HC-CDR3 20
ARWGLSWDGWGVTDY
SEQ ID NO: 159, HC-CDR3 21
ARYNYDT
SEQ ID NO: 160, HC-CDR3 22
ARSFGAGYDS
SEQ ID NO: 161, HC-CDR3 24
ARYPWDH
SEQ ID NO: 162, HC-CDR3 26
ARSSYYGYLSDG
SEQ ID NO: 163, LC-CDR1 20
QSISSY
SEQ ID NO: 164, LC-CDR1 21
TGTVTSTYY
SEQ ID NO: 165, LC-CDR1 22
SSDVGGYNY
SEQ ID NO: 166, LC-CDR1 26
SSNIGTNY
SEQ ID NO: 167, LC-CDR2 20
AAS
SEQ ID NO: 168, LC-CDR2 22
DVS
SEQ ID NO: 169, LC-CDR2 24
STN
SEQ ID NO: 170, LC-CDR3 20
QQSYSTPFT
SEQ ID NO: 171, LC-CDR3 21
LVFYGGVWV
SEQ ID NO: 172, LC-CDR3 22
SSYTSSSRYV
SEQ ID NO: 173, LC-CDR3 24
LLYYGGQGV
SEQ ID NO: 174, LC-CDR3 26
AAWDDSLSGLYV
SEQ ID NO: 175, LINKER 1
GGGGS
SEQ ID NO: 176, LINKER 2
SRGGGGSGGGGSGGGGSLEMA
SEQ ID NO: 177, CD28/CD3Z
AAAIEVMYPPPYLDNEKSNGTIIHVKGKHLCPSPLFPGPSKPFWVLVVVG
GVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRG
RDPEMGGKPRRKNPQEGLYNELQKDKMAEAYSEIGMKGERRRGKGHDGLY
QGLSTATKDTYDALHMQALPPR
SEQ ID NO: 178, 4-1BB/CD3Z
TGTTTPAPRPPTPAPTIASQPLSLRPEACRPAAGGAVHTRGLDFACDIYI
WAPLAGTCGVLLLSLVITLYCKRGRKKLLYIFKQPFMRPVQTTQEEDGCS
CRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHD
GLYQGLSTATKDTYDALHMQALPPR
SEQ ID NO: 179, HCVR 5
CAGGTCCAGCTGGTGCAGTCTGGAGCTGAGGTGAAGAAGCCTGGGGATTC
AGTGAAGGTCTCCTGCAAGCCTTCTGGTTACAATTTTCTCAACTATGGTA
TCAACTGGGTGCGACAGGCCCCTGGACAAGGGCTTGAGTGGATGGGATGG
ATTAGCACTTACACCGGTAACACAAACTATGCACAGAAGCTGCAGGGCAG
AGTCACCTTCACCACAGACACATCCACGAGCACAGCCTACATGGAGATGA
GGAGCCTGAGATCTGACGACACGGCCGTGTATTACTGTGCGCGCTCTTCT
GAATACTACACTTGGGATCATTGGGGTCAAGGTACTCTGGTGACCGTCTC
CTCA
SEQ ID NO: 180, LCVR 5
CAGTCTGTGTTGACGCAGCCGCCCTCAGTGTCTGGGGCCCCAGGGCAGAG
GGTCACCATCTCCTGCACTGGGAGCAGCTCCAACATCGGGGCAGGTTATG
ATGTACACTGGTACCAGCAGCTTCCAGGAACAGCCCCCAAACTCCTCATC
TATGGTAACAGCAATCGGCCCTCAGGGGTCCCTGACCGATTCTCTGGCTC
CAAGTCTGGCACCTCAGCCTCCCTGGCCATCACTGGGCTCCAGGCTGAGG
ATGAGGCTGATTATTACTGCCAGTCCTATGACAGCAGCCTGAGTGGTTGG
GTGTTCGGCGGAGGGACCAAGCTGACCGTCCTAGGT
SEQ ID NO: 181, HC-CDR3 consensus 2
A-R-Y-X-F/W/Y-D, wherein X is any amino acid
SEQ ID NO: 182, LC-CDR3 consensus 4
A/G-X-W-D/E-X-S-L, wherein X is any amino acid
SEQ ID NO: 183, LC-CDR3 consensus 5
L-L/V-F/Y-X-G-G, wherein Xis any amino acid
SEQ ID NO: 184, LC-CDR3 consensus 6
S/T-W/Y-X-S/T-S-L, wherein X is any amino acid

What is claimed is:

1. An isolated anti-PMC construct comprising an antibody moiety that specifically binds to a complex comprising a prostate-specific antigen (PSA) peptide and a major histocompatibility (MHC) class I protein (a PSA/MHC class I complex, or PMC).

2. The isolated anti-PMC construct of claim 1, wherein the MHC class I protein is the HLA-A*02:01 subtype of the HLA-A02 allele.

3. The isolated anti-PMC construct of claim 1 or 2, wherein the PSA peptide has an amino acid sequence selected from the group consisting of SEQ ID NOs: 3-13.

4. The isolated anti-PMC construct of claim 3, wherein the PSA peptide has the amino acid sequence of KLQCVDLHV (SEQ ID NO: 4).

5. The isolated anti-PMC construct of any one of claims 1-4, wherein the antibody moiety is a full-length antibody, a Fab, a Fab′, a (Fab′)2, an Fv, or a single chain Fv (scFv).

6. The isolated anti-PMC construct of any one of claims 1-5, wherein the isolated anti-PMC construct binds to the PSA/MHC class I complex with a K_{d} from about 0.1 pM to about 500 nM.

7. The isolated anti-PMC construct of any one of claims 1-6, wherein the antibody moiety comprises:
i) a heavy chain variable domain comprising a heavy chain complementarity determining region (HC-CDR) 1 comprising the amino acid sequence of G-G/Y-S/T-F-S/T-S-Y-A/G (SEQ ID NO: 118), or a variant thereof comprising up to about 3 amino acid substitutions, an HC-CDR2 comprising the amino acid sequence of I-X-P-X-X-G-X-T (SEQ ID NO: 119), or a variant thereof comprising up to about 3 amino acid substitutions, and an HC-CDR3 comprising the amino acid sequence of A-R-Y-X-F/W/Y-D (SEQ ID NO: 181); or a variant thereof comprising up to about 3 amino acid substitutions; and
ii) a light chain variable domain comprising a light chain complementarity determining region (LC-CDR) 1 comprising the amino acid sequence of S/T-S-N-F/I-G-A/N/S-G/N-Y (SEQ ID NO: 121), or a variant thereof comprising up to about 3 amino acid substitutions, and an LC-CDR3 comprising the amino acid sequence of A/G-X-W-D/E-X-S-L (SEQ ID NO: 182), L-L/V-F/Y-X-G-G (SEQ ID NO: 183), or S/T-W/Y-X-S/T-S-L (SEQ ID NO: 184); or a variant thereof comprising up to 3 amino acid substitutions, wherein
X can be any amino acid.

8. The isolated anti-PMC construct of any one of claims 1-6, wherein the antibody moiety comprises:
i) a heavy chain variable domain comprising an HC-CDR1 comprising the amino acid sequence of any one of SEQ ID NOs: 40-52, 155, and 156, or a variant thereof comprising up to about 5 amino acid substitutions, an HC-CDR2 comprising the amino acid sequence of any one of SEQ ID NOs: 53-65 and 157, or a variant thereof comprising up to about 5 amino acid substitutions, and an HC-CDR3 comprising the amino acid sequence of any one of SEQ ID NOs: 66-78 and 158-162; or a variant thereof comprising up to about 5 amino acid substitutions; and
ii) a light chain variable domain comprising an LC-CDR1 comprising the amino acid sequence of any one of SEQ ID NOs: 79-91 and 163-166, or a variant thereof comprising up to about 5 amino acid substitutions, an LC-CDR2 comprising the amino acid sequence of any one of SEQ ID NOs: 92-104 and 167-169, or a variant thereof comprising up to about 3 amino acid substitutions, and an LC-CDR3 comprising the amino acid sequence of any one of SEQ ID NOs: 105-117 and 170-174; or a variant thereof comprising up to about 5 amino acid substitutions.

9. The isolated anti-PMC construct of claim 8, wherein the antibody moiety comprises a) a heavy chain variable domain comprising the amino acid sequence of any one of SEQ ID NOs: 14-26 and 145-149, or a variant thereof having at least about 95% sequence identify to any one of SEQ ID NOs: 14-26 and 145-149; and b) a light chain variable domain comprising the amino acid sequence of any one of SEQ ID NOs: 27-39 and 150-154, or a variant thereof having at least about 95% sequence identity to any one of SEQ ID NOs: 27-39 and 150-154.

10. The isolated anti-PMC construct of any one of claims 1-9, wherein the isolated anti-PMC construct is multispecific.

11. The isolated anti-PMC construct of claim 10, wherein the isolated anti-PMC construct is a tandem scFv, a diabody (Db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody.

12. The isolated anti-PMC construct of claim 11, wherein the isolated anti-PMC construct is a tandem scFv comprising two scFvs linked by a peptide linker.

13. The isolated anti-PMC construct of any one of claims 10-12, wherein the isolated anti-PMC construct further comprises a second antibody moiety that specifically binds to a second antigen.

14. The isolated anti-PMC construct of claim 13, wherein the second antigen is selected from the group consisting of CD3γ, CD3δ, CD3ϵ, CD3ζ, CD28, OX40, GITR, CD137, CD27, CD40L, and HVEM.

15. The isolated anti-PMC construct of claim 13, wherein the second antigen is CD3ε, and wherein the isolated anti-PMC construct is a tandem scFv comprising an N-terminal scFv specific for the PSA/MHC class I complex and a C-terminal scFv specific for CD3ε.

16. The isolated anti-PMC construct of any one of claims 1-9, wherein the isolated anti-PMC construct is a chimeric antigen receptor comprising an extracellular domain comprising the antibody moiety, a transmembrane domain, and an intracellular signaling domain comprising a CD3ζ intracellular signaling sequence and a CD28 or 4-1BB intracellular signaling sequence.

17. The isolated anti-PMC construct of any one of claims 1-9, wherein the isolated anti-PMC construct is an immunoconjugate comprising the antibody moiety and an effector molecule, wherein the effector molecule is a therapeutic agent selected from the group consisting of a drug, a toxin, a radioisotope, a protein, a peptide, and a nucleic acid.

18. The isolated anti-PMC construct of any one of claims 1-9, wherein the isolated anti-PMC construct is an immunoconjugate comprising the antibody moiety and a label.

19. A nucleic acid encoding the polypeptide components of the isolated anti-PMC construct of any one of claims 1-18.

20. A pharmaceutical composition comprising the isolated anti-PMC construct of any one of claims 1-17 or the nucleic acid of claim 19.

21. A host cell expressing the isolated anti-PMC construct of any one of claims 1-18.

22. An effector cell expressing the isolated anti-PMC construct of claim 16.

23. The effector cell of claim 22, wherein the effector cell is a T cell.

24. A method of detecting a cell presenting a complex comprising a PSA peptide and an MHC class I protein on its surface, comprising contacting the cell with the isolated anti-PMC construct of claim 18 and detecting the presence of the label on the cell.

25. A method of treating an individual having a PSA-positive disease, comprising administering to the individual
a) an effective amount of the pharmaceutical composition of claim 20, or
b) an effective amount of the effector cell of claim 22 or 23.

26. A method of diagnosing an individual having a PSA-positive disease, comprising:
a) administering an effective amount of the isolated anti-PMC construct of claim 18 to the individual; and
b) determining the level of the label in the individual, wherein a level of the label above a threshold level indicates that the individual has the PSA-positive disease.

27. A method of diagnosing an individual having a PSA-positive disease, comprising:
a) contacting a sample derived from the individual with the isolated anti-PMC construct of claim 18; and
b) determining the number of cells bound with the isolated anti-PMC construct in the sample, wherein a value for the number of cells bound with the isolated anti-PMC construct above a threshold level indicates that the individual has the PSA-positive disease.

28. The method of any one of claims 25-27, wherein the PSA-positive disease is cancer.

29. The method of claim 28, wherein the cancer is selected from the group consisting of prostate cancer, breast cancer, ovarian cancer, and lung cancer.

30. The method of claim 29, where the cancer is prostate cancer.

31. The method of claim 30, wherein the prostate cancer is hormone-resistant prostate cancer.
